(19) United States
(12) Patent Application Publication
(10) Pub. No.: US 2008/0255085 A1
(43) Pub. Date: Oct. 16, 2008

(54) Novel Imidazo [4,5-b] Pyridine Derivatives as Inhibitors of Glycogen Synthase Kinase 3 for Use in the Treatment of Dementia and Neurodegenerative Disorders

(75) Inventors: Per I Arvidsson, Sodertalje (SE); Erwan Arzel, Sodertalje (SE); Jeremy Burrows, Sodertalje (SE); Martina Claesson, Sodertalje (SE); Colin Ray, Sodertalje (SE); Tobias Rein, Sodertalje (SE); Didier Rotticci, Sodertalje (SE); Peter Soderman, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, SODERTALJE (SE)

(21) Appl. No.: 12/089,002

(22) PCT Filed: Oct. 2, 2006
(86) PCT No.: PCT/SE06/01114

§ 371 Date: Apr. 2, 2008

(30) Foreign Application Priority Data
Oct. 3, 2005 (SE) 0502172-0

(51) Int. Cl. A61K 31/5377; C07D 487/04; A61K 31/496; A61P 25/16; A61P 25/28; A61K 31/437; A61K 31/551

(52) U.S. Cl. 514/210.18; 544/362; 514/253.04; 514/303; 546/118; 540/575; 514/218; 544/127

(57) ABSTRACT

Compounds of formula I

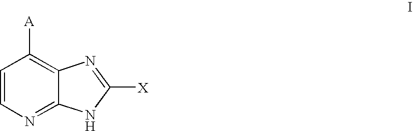

wherein X is

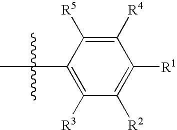

or Y;
and wherein A, Y, R¹, R², R³, R⁴ and R⁵ are as defined in the specification as a base or a pharmaceutically acceptable salt, solvate or solvate of salt thereof, processes for their preparation, new intermediates used therein, pharmaceutical formulations containing said compounds and to the use of said compounds in therapy.

### TECHNICAL FIELD OF THE INVENTION

The present invention relates to new compounds of formula I, as a free base or a pharmaceutically acceptable salt, solvate or solvate of salt thereof, to pharmaceutical formulations containing said compounds and to the use of said compounds in therapy. The present invention further relates to a process for the preparation of compounds of formula I and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous is system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but they are believed to be related. Glycogen synthase kinase 3β (GSK3β) or Tau (τ) phosphorylating kinase selectively phosphorylates the microtubule associated protein T in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of u and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K /Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3P may therefore be of therapeutic relevance in the treatment of BD as well as in AD patients that have affective disorders.

### Schizophrenia

GSK3 is involved in signal transduction cascades of multiple cellular processes, particularly during neural development. Kozlovsky et al (Am J Psychiatry 2000 May;157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February;49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

### Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25;95 (5):605-14)). The new follicles formed sebaceous glands and dermal papilla, normally established only in embryogenesis. Thus GSK3 inhibition may offer treatment for baldness.

### Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### Bone-Related Disorders

It has been shown that GSK3 inhibitors could be used for treatment of bone-related disorders. This has been discussed in e.g. Tobias et al., Expert Opinion on Therapeutic Targets, February 2002, pp 41-56.

### DISCLOSURE OF THE INVENTION

The object of the present invention is to provide compounds having a selective inhibiting effect at GSK3 as well as having a good bioavailability. Accordingly, the present invention provides a compound of the formula I:

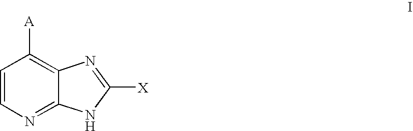

wherein

X is

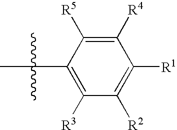

### or Y;

R¹ is selected from hydrogen, halogen, CN, CO₂H, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};

R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};

R³ and R⁵ are independently selected from hydrogen, C₁₋₃alkyl and C₁₋₃haloalkyl;

A is aryl or heteroaryl, optionally substituted with one or more CN, CO₂H, C₁₋₆alkyl, C₁₋₆haloalkyl, halo, C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};

Y is selected from Z, C₁₋₆alkyl, CH₂OR^{d}, and CH₂Z;

Z is heteroaryl optionally substituted with one or more CN, C₁₋₆alkyl C₁₋₆haloalkyl, halo, C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{e} are independently selected from hydrogen, heteroaryl, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl or C₁₋₃haloalkyl, wherein said C₁₋₃alkyl or C₁₋₃haloalkyl is optionally further substituted with one or more C₁₋₃alkoxy;

R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or

R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloallyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, said C₁₋₃alkyl or C₁₋₃haloalkyl, optionally substituted with one or more C₁₋₃alkoxy;

Rⁱ is C₁₋₃alkyl or C₁₋₃haloalkyl, said C₁₋₃alkyl or C₁₋₃haloalkyl optionally substituted with one or more OR^{a};

R^{j} is aryl or heteroaryl, wherein said aryl or heteroaryl is optionally substituted with one or more C₁₋₃alkyl, OR^{a}, halo or CN;

R^{k} is C₁₋₆alkyl or C₁₋₆haloalkyl, wherein said C₁₋₆alkyl or C₁₋₆haloalkyl is optionally substituted with at least one CN, OR^{a}, NR^{b}R^{c}, C(O)NR^{b}R^{c} or NR^{b}C(O)R^{c};

R^{m} is C₁₋₃alkyl, optionally substituted with at least one halo, CN, OR^{a}, NR^{b}R^{c} or C(O)NR^{b}R^{c};

n is 0 to 2;

as a free base or a pharmaceutically acceptable salt, solvate or solvate of a salt thereof.

The present invention also relates to a compound of the formula I:

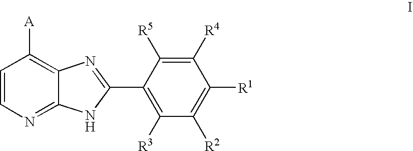

wherein

R¹ is hydrogen, halogen, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR, SO₂Rⁱ or C(O)R^{j};

R² and R⁴ are independently selected from hydrogen, halo, CN, NO₂, C₁₋₃alkyl, C₁₋₃haloalkyl, ORE, SO₂NR^{b}R^{c}, C(O)NR^{b}R^{c}, CH₂NR^{b}R^{c}, CH₂OR^{h}, SO₂Rⁱ and C(O)R^{j};

R³ and R⁵ are independently selected from hydrogen, C₁₋₃alkyl and C₁₋₃haloalkyl;

A is aryl or heteroaryl, optionally substituted with one or more CN, C₁₋₆alkyl, C₁₋₆haloalkyl halo, OR^{k}, C(O)NR^{b}R^{c} or S(O)ₙR^{m}, said C₁₋₆alkyl or C₁₋₆haloalkyl optionally substituted by at least one OR^{a} or NR^{b}R^{c};

R^{a} is hydrogen, C₁₋₃alkyl or C₁₋₃haloalkyl, said C₁₋₃alkyl or C₁₋₃haloalkyl optionally substituted with one or more C₁₋₃alkoxy;

R^{b} and R^{c} are independently selected from hydrogen, C₁₋₆alkyl and C₁₋₆haloalkyl, wherein said C₁₋₆alkyl or C₁₋₆haloalkyl optionally substituted with one or more OR^{a} or NR^{d}R^{e} or

R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;

R^{d} and R^{e} are independently selected from hydrogen, C₁₋₆alkyl or C₁₋₆haloalkyl, said C₁₋₆alkyl or C₁₋₆haloalkyl optionally substituted with one or more OR^{a}; or

R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;

R^{h} is hydrogen, C₁₋₃alkyl or C₁₋₃haloalkyl, said C₁₋₃alkyl or C₁₋₃haloalkyl optionally substituted with one or more C₁₋₃alkoxy;

Rⁱ is C₁₋₃alkyl or C₁₋₃haloalkyl, said C₁₋₃alkyl or C₁₋₃haloalkyl optionally substituted with one or more OR^{a};

R^{j} is aryl or heteroaryl, wherein said aryl or heteroaryl is optionally substituted with one or more C₁₋₃alkyl, OR^{a}, halo or CN;

R^{k} is C₁₋₆alkyl or C₁₋₆haloalkyl, optionally substituted with at least one CN, OR^{a}, NR^{b}R^{e} or C(O)NR^{b}R^{c};

R^{m} is C₁₋₃alkyl, optionally substituted with at least one halo, CN, OR^{a}, NR^{b}R^{c} or C(O)NR^{b}R^{c};

n is 0 to 2;

as a free base or a pharmaceutically acceptable salt, solvate or solvate of a salt thereof.

One embodiment of the present invention provides a compound, wherein R¹ is hydrogen, halogen, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ or C(O)R^{j};

R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};

R³ and R⁵ are independently selected from hydrogen, C₁₋₃alkyl and C₁₋₃haloalkyl;

A is phenyl or pyridyl, optionally substituted with one or more CN, C₁₋₆alkyl, C₁₋₆haloalkyl, halo, OR^{k}, C(O)NR^{b}R^{c} or S(O)ₙR^{m}, wherein said C₁₋₆alkyl or C₁₋₆haloalkyl is optionally substituted by at least one OR^{a} or NR^{b}R^{c};

R^{a} is hydrogen, C₁₋₃alkyl or C₁₋₃haloalkyl, said C₁₋₃alkyl or C₁₋₃haloalkyl optionally substituted with one or more C₁₋₃alkoxy;

R^{b} and R^{c} are independently selected from hydrogen, C₁₋₆alkyl and C₁₋₆haloalkyl, wherein said C₁₋₆alkyl or C₁₋₆haloalkyl is optionally substituted with one or more OR^{a} or NR^{d}R^{e} or

R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or

R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, said C₁₋₃alkyl or C₁₋₃haloalkyl optionally substituted with one or more C₁₋₃alkoxy;

Rⁱ is C₁₋₃alkyl or C₁₋₃haloalkyl, wherein said C₁₋₃alkyl or C₁₋₃haloalkyl is optionally substituted with one or more OR^{a};

Rⁱ is aryl or heteroaryl, wherein said aryl or heteroaryl is optionally substituted with one or more C₁₋₃alkyl, OR^{a}, halo or CN;

R^{k} is C₁₋₆alkyl or C₁₋₆haloalkyl, optionally substituted with at least one CN, OR^{a}, NR R^{c} or C(O)NR^{b}R^{c};

R^{m} is C₁₋₃alkyl, optionally substituted with at least one halo, CN, OR^{a}, NR^{b}R^{c} or C(O)NR^{b}R^{c};

n is 0 to 2;

as a free base or a pharmaceutically acceptable salt, solvate or solvate of a salt thereof.

Another embodiment of the present invention provides a compound of the formula I, wherein

R¹ is hydrogen, SO₂NR^{b}R^{c}, C(O)NR^{b}R^{c}, CH₂NR^{b}R^{c}, CH₂OR^{h} or SO₂Rⁱ;

R² and R⁴ are independently selected from hydrogen, halo, CN, NO₂, C₁₋₃alkyl, C₁₋₃haloalkyl, OR^{a}, C(O)NR^{b}R^{c}, CH₂NR^{b}R^{c}, CH₂OR^{h} and SO₂Rⁱ;

R³ and R⁵ are hydrogen;

A is phenyl or pyridyl, optionally substituted with one or more CN, C₁₋₆alkyl, halo, OR^{k} or C(O)NR^{b}R^{c}, said C₁₋₆alkyl optionally substituted by at least one OR^{a} or NR^{b}R^{c};

R^{a} is C₁₋₃alkyl or C₁₋₃haloalkyl, said C₁₋₃alkyl or C₁₋₃haloalkyl optionally substituted with one or more C₁₋₃alkoxy;

R^{b} and R^{c} are independently selected from hydrogen, C₁₋₆alkyl or C₁₋₆haloalkyl, wherein said C₁₋₆alkyl or C₁₋₆haloalkyl is optionally substituted with one or more OR^{a} or NR^{d}R^{e} or

R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{d} and R^{e} form, together with the atom to which they are attached, a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl;

Rⁱ is C₁₋₃alkyl or C₁₋₃haloalkyl;

R^{k} is C₁₋₆alkyl or C₁₋₆haloalkyl, optionally substituted with at least one CN, OR^{a}, NR^{b}R^{c} or C(O)NR^{b}R^{c};

as a free base or a pharmaceutically acceptable salt, solvate or solvate of a salt thereof.

A further embodiment of the present invention relates to a compound of the formula I, wherein

R¹ is SO₂NR^{b}R^{c}, C(O)NR^{b}R^{c} or CH₂NR^{b}R^{c};

R², R³, R⁴ and R⁵ are hydrogen;

A is phenyl or pyridyl, optionally substituted with one or more CN, C₁₋₆alkyl, halo, OR^{k} or C(O)NR^{b}R^{c}, wherein said C₁₋₆alkyl is optionally substituted by at least one NR^{b}R^{c};

R^{b} and R^{c} are independently selected from hydrogen or C₁₋₆alkyl, wherein said C₁₋₆allyl is optionally substituted with one or more NR^{d}R^{e} or

R^{b} and R^{c} may, together with the atom to which they are attached, form a 6-membered heterocyclic ring containing one or more heteroatoms selected from N or O, wherein said heterocyclic ring is optionally substituted with one or more C₁₋₃alkyl;

R^{d} and R^{e} form, together with the atom to which they are attached, a 6-membered heterocyclic ring containing one or more heteroatoms selected from N, O or S;

R^{k} is C₁₋₆alkyl or C₁₋₆haloalkyl;

as a free base or a pharmaceutically acceptable salt, solvate or solvate of a salt thereof.

Yet another embodiment of the present invention relates to a compound of the formula I, wherein

R¹ is selected from hydrogen, halogen, CN, CO₂H, NO₂, OR^{a}, SO₂NR^{b}R^{c}, C(O)NR^{b}R^{c}, CH₂NR^{b}R^{c}, CH₂OR^{h}, SO₂Rⁱ and C(O)R^{j};

R² and R⁴ are independently selected from hydrogen, halo, CN, NO₂, OR^{a}, SO₂NR^{b}R^{c}, C(O)NR^{b}R^{c}, CH₂NR^{b}R^{c}, CH₂OR^{h}, SO₂Rⁱ and C(O)R^{j};

R³ and R⁵ are independently selected from hydrogen, C₁₋₃allyl and C₁₋₃haloalkyl;

A is aryl or heteroaryl, optionally substituted with one or more CN, CO₂H, C₁₋₆alkyl, C₁₋₆haloalkyl, halo,C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};

Y is selected from Z, C₁₋₆alkyl, CH₂OR^{d}, and CH₂Z;

Z is heteroaryl optionally substituted with one or more CN, C₁₋₆alkyl C₁₋₆haloalkyl, halo, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{c} are independently selected from hydrogen, heteroaryl, C₁₋₆alkyl and C₁₋₆haloalkyl, wherein said C₁₋₆alkyl or C₁₋₆haloalkyl optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;

R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or

R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃allyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, said C₁₋₃alkyl or C₁₋₃haloalkyl, optionally substituted with one or more C₁₋₃alkoxy;

Rⁱ is C₁₋₃alkyl or C₁₋₃haloalkyl, said C₁₋₃alkyl or C₁₋₃haloallyl optionally substituted with one or more OR^{a};

R^{j} is aryl or heteroaryl, wherein said aryl or heteroaryl is optionally substituted with one or more C₁₋₃alkyl, OR^{a}, halo or CN;

R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};

R^{m} is C₁₋₃alkyl, optionally substituted with at least one halo, CN, OR^{a}, NR^{b}R^{c} or C(O)NR^{b}R^{c};

n is 0 to 2;

as a free base or a pharmaceutically acceptable salt, solvate or solvate of a salt thereof.

A further embodiment of the present invention provides a compound of the formula I,

wherein R¹ is selected from hydrogen, halogen, CO₂H, NO₂, OR^{a}, SO₂NR^{b}R^{c}, C(O)NR^{b}R^{c}, CH₂NR^{b}R^{c}, CH₂OR^{h}, and SO₂Rⁱ;

R² and R⁴ are independently selected from hydrogen, halo, OR^{a}, SO₂NR^{b}R^{c}, C(O)NR^{b}R^{c}, CH₂NR^{b}R^{c}, CH₂OR^{h}, and SO₂Rⁱ;

R³ and R⁵ are independently selected from hydrogen, C₁₋₃alkyl and C₁₋₃haloalkyl;

A is aryl or heteroaryl, optionally substituted with one or more CN, CO₂H, C₁₋₆alkyl, C₁₋₆haloalkyl, halo,C(O)R^{a}, OR^{k} or C(O)NR^{b}R^{c}, wherein said C₁₋₆alkyl or C₁₋₆haloalkyl is optionally substituted by at least one CN, OR^{a} or NR^{b}R^{c};

Y is selected from Z, C₁₋₆alkyl, CH₂OR^{d}, and CH₂Z;

Z is heteroaryl optionally substituted with one or more CN, C₁₋₆alkyl, C₁₋₆haloalkyl, halo, C(O)NR^{b}R^{c} or S(O)ₙR^{m} , wherein said C₁₋₆alkyl or C₁₋₆haloalkyl is optionally substituted by at least one CN, OR^{a} or NR^{b}R^{c};

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{c} are independently selected from hydrogen, heteroaryl, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;

R^{d} and R^{e} are independently selected from hydrogen, C₁₋₆alkyl or C₁₋₆haloalkyl, said C₁₋₆alkyl or C₁₋₆haloalkyl optionally substituted with one or more OR^{a}; or

R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃allyl or C₁₋₃haloalkyl, said C₁₋₃alkyl or C₁₋₃haloalkyl, optionally substituted with one or more C₁₋₃alkoxy;

Rⁱ is C₁₋₃alkyl or C₁₋₃haloallyl, said C₁₋₃alkyl or C₁₋₃haloalkyl optionally substituted with one or more OR^{a};

R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};

R^{m} is C₁₋₃alkyl, optionally substituted with at least one halo, CN, OR^{a}, NR^{b}R^{c} or C(O)NR^{b}R^{c};

as a free base or a pharmaceutically acceptable salt, solvate or solvate of a salt thereof.

Another embodiment of the present invention provides a compound of the formula I,

wherein R¹ is selected from hydrogen, CO₂H, SO₂NR^{b}R^{c}, C(O)NR^{b}R^{c}, CH₂NR^{b}R^{c}, and SO₂Rⁱ;

R² and R⁴ are independently selected from hydrogen, C(O)NR^{b}R^{c}, CH₂NR^{b}R^{c}, and SO₂Rⁱ;

R³ and R⁵ are hydrogen;

A is aryl or heteroaryl, optionally substituted with one or more CN, CO₂H, C₁₋₆alkyl , halo, C(O)R^{a}, OR^{k}, C(O)NR^{b}R^{c} or S(O)ₙR^{m}, wherein said C₁₋₆alkyl is optionally substituted by at least one CN, OR^{a} or NR^{b}R^{c};

Y is selected from Z, C₁₋₆alkyl, CH₂OR^{d}, and CH₂Z;

Z is heteroaryl optionally substituted with one or more CN, C₁₋₆alkyl or C(O)NR^{b}R^{c};

R^{a} is selected from hydrogen and C₁₋₃alkyl, wherein said C₁₋₃alkyl is optionally substituted with one or more C₁₋₃alkoxy;

R^{b} and R^{c} are independently selected from hydrogen, heteroaryl and C₁₋₆alkyl, wherein said C₁₋₆alkyl is optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;

R^{d} and R^{e} are, C₁₋₆alkyl; or

R^{d} and R^{e} may, together with the atom to which they are attached, form a 4-, 5-, 6- or 7-membered heterocyclic ring containing one or more heteroatoms selected from N or O;

Rⁱ is C₁₋₃alkyl;

R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};

as a free base or a pharmaceutically acceptable salt, solvate or solvate of a salt thereof.

One embodiment of the present invention provides a compound of the formula I, wherein A is phenyl or pyridyl.

Yet another embodiment of the present invention relates to a compound of the formula I, wherein R³ and R⁵ is hydrogen.

A further embodiment of the present invention provides a compound of the formula I, wherein A is heteroaryl.

Another embodiment of the present invention provides a compound of the formula I, wherein A is pyridyl.

According to one embodiment, the present invention also relates to a compound of the formula I, wherein A is aryl, optionally substituted with one or more CN, CO₂H, C₁₋₆alkyl, C₁₋₆haloalkyl, halo, C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};

Another embodiment of the present invention relates to compound of the formula I, wherein said aryl is phenyl.

One additional embodiment of the present invention provides a compound of the formula I, wherein A is substituted with one or more CN, CO₂H, C₁₋₆alkyl, halo,C(O)R^{a}, OR^{k} or C(O)NR^{b}R^{c}, wherein said C₁₋₆alkyl is optionally substituted by at least one CN, OR^{a} or NR^{b}R^{c};

One embodiment of the present invention relates to a compound of the formula I, wherein A is substituted with OR^{k}, C₁₋₆alkyl, halo or C(O)NR^{b}R^{c}.

A further embodiment of the present invention relates to a compound of the formula I, wherein A is substituted with OR^{k} and R^{k} is C₁₋₆alkyl. According to one embodiment of the present invention R^{k} is methyl.

Yet a further embodiment of the present invention relates to a compound of the formula I, wherein R¹ and R² are hydrogen; R⁴ is C(O)NR^{b}R^{c};

R^{b} and R^{c} are independently selected from hydrogen, heteroaryl and C₁₋₆alkyl, wherein said C₁₋₆alkyl is optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N or O, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;

R^{a} is C₁₋₃allyl, wherein said C₁₋₃alkyl is optionally substituted with one or more C₁₋₃alkoxy; and

R^{d} and R^{e} may, together with the atom to which they are attached, form a 5-membered heterocyclic ring containing one or more heteroatoms selected from N.

One embodiment of the present invention provides a compound of the formula I, wherein R¹ and R⁴ are hydrogen; R² is SO₂Rⁱ; and R^{e} is C₁₋₃alkyl or C₁₋₃haloalkyl.

Yet another embodiment of the present invention relates to a compound of the formula I, wherein Rⁱ is methyl.

A further embodiment of the present invention provides a compound of the formula I, wherein R² and R⁴ are hydrogen; A is substituted with one or more halo, OR^{k} or C(O)NR^{b}R^{c} and wherein R^{k} is C₁₋₆ alkyl; and

R^{b} and R^{c} together with the atom to which they are attached, form a 4-, 5- or 6-membered heterocyclic ring containing one or more heteroatoms selected from N or O, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy.

According to one embodiment of the present invention there is provided a compound of the formula I, wherein A is substituted with OR^{k} or C(O)NR^{b}R^{c}

Another embodiment of the present invention relates to a compound of the formula I, wherein R^{k} is C₁₋₆alkyl. According to one additional embodiment of the present invention, R^{k} is methyl.

Yet another embodiment of the present invention provides a compound of the formula I, wherein

R^{b} and R^{c} are independently selected from hydrogen, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl or C₁₋₃haloalkyl, wherein said C₁₋₃alkyl or C₁₋₃haloalkyl is optionally further substituted with one or more C₁₋₃alkoxy.

Another embodiment of the present invention providesa compound of the formula I, wherein R^{b} and R^{c} together with the atom to which they are attached, form a 5-, 6- or 7-membered heterocyclic ring containing one or more heteroatoms selected from N or O, wherein said heterocyclic ring is optionally substituted with one or more halo or C₁₋₃alkyl, wherein said C₁₋₃alkyl is optionally further substituted with one or more C₁₋₃alkoxy.

One additional embodiment of the present invention relates to a compound of the formula I, wherein R¹ is selected from halogen, CO₂H, C(O)NR^{b}R^{c} and CH₂NR^{b}R^{c}.

Yet one additional embodiment of the present invention provides a compound of the formula I, wherein

R¹ is C(O)NR^{b}R^{c} or CH₂NR^{b}R^{c}; and

R^{b} and R^{c} together with the atom to which they are attached, form a 5-, 6- or 7-membered heterocyclic ring containing one or more heteroatoms selected from N or O, wherein said heterocyclic ring is optionally substituted with one or more halo or C₁₋₃alkyl, wherein said C₁₋₃alkyl is optionally further substituted with one or more C₁₋₃alkoxy.

The present invention also relates to a compound selected from:

7-(4-Methoxyphenyl)-2-{4-[(4-methylpiperazin-1-yl)sulfonyl]phenyl}-3H-imidazo[4,5-b]pyridine hydrochloride;

7-(3-Methoxyphenyl)-2-{4-[(4-methylpiperazin-1-yl)sulfonyl]phenyl}-3H-imidazo[4,5-b]pyridine hydrochloride;

7-(4-Methoxyphenyl)-2-{4-[(4-methylpiperazin-1-yl)carbonyl]phenyl}-3H-imidazo[4,5-b]pyridine hydrochloride;

7-(3-methoxyphenyl)-2-{4-[(4-methylpiperazin-1-yl)carbonyl]phenyl}-3H-imidazo[4,5-b]pyridine hydrochloride;

7-(4-Chlorophenyl)-2-{4-[(4-methylpiperazin-1-yl)carbonyl]phenyl}-3H-imidazo[4,5-b]pyridine hydrochloride;

7-(4-Methoxyphenyl)-2-[4-(piperidin-1-ylmethyl)phenyl]-3H-imidazo[4,5-b]pyridine hydrochloride;

4-[7-(4-Methoxyphenyl)-3H-imidazo[4,5-b]pyridin-2-yl]-N-(2-morpholin-4-ylethyl)benzamide hydrochloride;

2-{4-[(4-Methylpiperazin-1-yl)carbonyl]phenyl}-7-[4-(trifluoromethoxy)phenyl]-3H-imidazo[4,5-b]pyridine hydrochloride;

2-{4-[(4-Methylpiperazin-1-yl)carbonyl]phenyl}-7-pyridin-3-yl-3H-imidazo[4,5-b]pyridine hydrochloride;

7-(2,4-Dimethoxyphenyl)-2-[4-(morpholin-4-ylcarbonyl)phenyl]-3H-imidazo[4,5-b]pyridine hydrochloride;

4-(2-{4-[(4-Methylpiperazin-1-yl)carbonyl]phenyl}-3H-imidazo[4,5-b]pyridin-7-yl)benzonitrile hydrochloride;

7-(4-Methoxyphenyl)-2-[4-(morpholin-4-ylmethyl)phenyl]-3H-imidazo[4,5-b]pyridine hydrochloride;

2-{4-[4-Methylpiperazin-1-yl)carbonyl]phenyl}-7-[3-(morpholin-4-ylmethyl)phenyl]-1H-imidazo[4,5-b]pyridine;

N-(2-Cyanoethyl)-3-{2-[(4-methylpiperazin-1-yl)carbonyl]-3H-imidazo[4,5-b]pyridine-7-yl}benzamide;

7-{3-[2-(2-Methoxyethoxy)ethoxy]phenyl}-2-{4-[(4-methylpiperazin-1-yl)carbonyl]phenyl)-3H-imidazo[4,5-b]pyridine;

3-[3-(2-{4-[(4-Methylpiperazin-1-yl)carbonyl]phenyl}-3H-imidazo[4,5-b]pyridin-7-yl)propan-1-ol;

7-[3-(3-Methoxypropoxy)phenyl]-2-{4-[(4-methylpiperazin-1-yl)carbonyl]phenyl}-3H-imidazo[4,5-b]pyridine;

N-{3-[3-(2-{4-[4-Methylpiperazin-1-yl)carbonyl]phenyl}-3H-imidazo[4,5-b]pyridine-7-yl)phenoxy]propyl}acetamide;

4-[3-(2-{4-[(4-Methylpiperazin-1-yl)carbonyl]phenyl}-3H-imidazo[4,5-b]pyridine-7-yl)phenoxy]butanenitrile;

3-[3-(2-{4-[(4-Methylpiperazin-1-yl)carbonyl]phenyl}-3H-imidazo[4,5-b]pyridine-7-yl)phenoxy]propan-1-ol;

3-[3-(2-{4-[(4-Methylpiperazin-1-yl)carbonyl]phenyl}-3H-imidazo[4,5-b]pyridine-7-yl)phenoxy]acetonitrile;

7-(4-Methoxyphenyl)-2-{5-[(4-methylpiperzin-1-yl)carbonyl]pyridine-2-yl}-3H-imidazo[4,5-b]pyridine;

2-{4-[(3,3-Difluoropyrrolidin-1-yl)carbonyl]phenyl}-7-(4-methoxyphenyl)3H-imidazo[4,5-b]pyridine;

7-(4-Methoxyphenyl)-2-(4-{[(3R)-3-methylmorpholin-4-yl]carbonyl}phenyl)-3H-imidazo[4,5-b]pyridine;

2-{4-[(4-Ethylpiperazin-1-yl)carbonyl]phenyl}-7-(4-methoxyphenyl)-3H-imidazo[4,5-b]pyridine;

4-[7-(4-Methoxyphenyl)-3H-imidazo[4,5-b]pyridin-2-yl]-N-(2-piperidin-1-ylethyl)benzamide;

7-(4-Methoxyphenyl)-2-{4-[(4-methyl-1,4-diazepan-1-yl)carbonyl]phenyl}-3H-imidazo[4,5-b]pyridine;

1-{4-[7-(4-Methoxyphenyl)-3H-imidazo[4,5-b]pyridin-2-yl]benzoyl}-N,N-dimethylpyrrolidin-3-amine;

2-(4-{[4-(2-Methoxyethyl)piperazin-1-yl]carbonyl}phenyl)-7-(4-methoxyphenyl)-3H-imidazo[4,5-b]pyridine;

2-{4-[(4-Isopropylpiperazin-1-yl)carbonyl]phenyl}-7-(4-methoxyphenyl)-3H-imidazo[4,5-b]pyridine;

2-(4-{[(3 S)-3-Fluoropyrrolidin-1-yl]carbonyl}phenyl)-7-(4-methoxyphenyl)-3H-imidazo[4,5-b]pyridine;

1-{4-[7-(4-Methoxyphenyl)-3H-imidazo[4,5-b]pyridin-2-yl]benzoylpyrrolidin-3-ol hydrochloride;

7-(3-Fluoro-4-Methoxyphenyl)-2-{4-[(4-methylpiperazin-1-yl)carbonyl]phenyl}-3H-imidazo[4,5-b]pyridine hydrochloride;

7-(4-Isopropoxyphenyl)-2-{4-[(4-methylpiperazin-1-yl)carbonyl]phenyl}-3H-imidazo[4,5-b]pyridine hydrochloride;

7-(4-Ethoxyphenyl)-2-{4-[(4-methylpiperazin-1-yl)carbonyl]phenyl}-3H-imidazo[4,5-b]pyridine hydrochloride;

7-(4-Methoxy-2-methylphenyl)-2-{4-[(4-methylpiperazin-1-yl)carbonyl]phenyl}-3H-imidazo[4,5-b]pyridine hydrochloride;

7-(4-Methoxyphenyl)-2-{2-[(4-methylpiperazin-1-yl)carbonyl]pyridin-4-yl}-3H-imidazo[4,5-b]pyridine;

7-(4-Methoxyphenyl)-2-{5-[(4-methylpiperazin-1-yl)carbonyl]pyridin-3-yl}-3H-imidazo[4,5-b]pyridine;

7-(2-Methoxyphenyl)-2-[4-(morpholin-4-ylcarbonyl)phenyl]-3H-imidazo[4,5-b]pyridine hydrochloride;

N-(3-Methoxypropyl)-4-(7-pyridin-4-yl-3H-imidazo[4,5-b]pyridin-2-yl)benzamide hydrochloride;

2-{4-[(4-Methylpiperazin-1-yl)carbonyl]phenyl}-7-pyridin-4-yl-3H-imidazo[4,5-b]pyridine hydrochloride;

2-{4-[(4-Methylpiperazin-1-yl)methyl]phenyl}-7-pyridin-4-yl-3H-imidazo[4,5-b]pyridine hydrochloride;

4-(2-{4-[(4-Methylpiperazin-1-yl)methyl]phenyl}-3H-imidazo[4,5-b]pyridin-7-yl)benzamide hydrochloride;

7-(4-Methoxyphenyl)-2-{4-[(4-methylpiperazin-1-yl)methyl]phenyl}-3H-imidazo[4,5-b]pyridine hydrochloride;

7-(4-Ethoxyphenyl)-2-{4-[(4-methylpiperazin-1-yl)methyl]phenyl}-3H-imidazo[4,5-b]pyridine hydrochloride;

(4-{2-[4-(Morpholin-4-ylmethyl)phenyl]-3H-imidazo[4,5-b]pyridin-7-yl}phenyl)methanol hydrochloride;

N-Methyl-4-{2-[4-(morpholin-4-ylmethyl)phenyl]-3H-imidazo[4,5-b]pyridin-7-yl}benzamide hydrochloride;

2-[4-(Morpholin-4-ylmethyl)phenyl]-7-[4-(pyrrolidin-1-ylcarbonyl)phenyl]-3H-imidazo[4,5-b]pyridine hydrochloride;

4-{2-[4-(Morpholin-4-ylmethyl)phenyl]-3H-imidazo[4,5-b]pyridin-7-yl}benzamide hydrochloride;

(4-{2-[4-(Morpholin-4-ylmethyl)phenyl]-3H-imidazo[4,5-b]pyridin-7-yl}phenyl)acetonitrile hydrochloride;

4-{2-[4-(Morpholin-4-ylethyl)phenyl]-3H-imidazo[4,5-b]pyridin-7-yl}benzoic acid hydrochloride;

4,4′-(3H-Imidazo[4,5-b]pyridine-2,7-diyl)dibenzoic acid;

7-[4-(Azetidin-1-ylcarbonyl)phenyl]-2-[4-(morpholin-4-ylmethyl)phenyl]-3H-imidazo[4,5-b]pyridine hydrochloride;

1-(4-{2-[4-(Morpholin-4-ylmethyl)phenyl]-3H-imidazo[4,5-b]pyridin-7-yl}phenyl)ethanone hydrochloride;

7-(4-Methoxyphenyl)-2-[3-(morpholin-4-ylmethyl)phenyl]-3H-imidazo[4,5-b]pyridine hydrochloride;

7-(4-Methoxyphenyl)-2-{3-[(4-methylpiperazin-1-yl)methyl]phenyl}-3H-imidazo[4,5-b]pyridine hydrochloride;

7-(4-Methoxyphenyl)-2-phenyl-3H-imidazo[4,5-b]pyridine;

7-(4-Methoxyphenyl)-2-[3-(methylsulfonyl)phenyl]-3H-imidazo[4,5-b]pyridine;

7-(4-Methoxyphenyl)-2-[4-(methylsulfonyl)phenyl]-3H-imidazo[4,5-b]pyridine;

7-(4-Methoxyphenyl)-2-(1H-pyrrol-2-yl)-3H-imidazo[4,5-b]pyridine;

7-(4-Methoxyphenyl)-2-pyridazin-4-yl-3H-imidazo[4,5-b]pyridine;

5-[7-(4-Methoxyphenyl)-3H-imidazo[4,5-b]pyridin-2-yl]pyridine-2-carbonitrile;

7-(4-Methoxyphenyl)-2-(6-methylpyridin-3-yl)-3H-imidazo[4,5-b]pyridine;

7-(4-Methoxyphenyl)-2-(1-methylcyclopropyl)-3H-imidazo[4,5-b]pyridine;

2-(2-Furylmethyl)-7-(4-methoxyphenyl)-3H-imidazo[4,5-b]pyridine;

2-(Butoxymethyl)-7-(4-methoxyphenyl)-3H-imidazo[4,5-b]pyridine;

2-(Methoxymethyl)-7-(4-methoxyphenyl)-3H-imidazo[4,5-b]pyridine;

3-[7-(4-Methoxyphenyl)-3H-imidazo[4,5-b]pyridin-2-yl]-N-(3-methoxypropyl)benzamide hydrochloride;

3-{7-[4-(Aminocarbonyl)phenyl]-3H-imidazo[4,5-b]pyridin-2-yl}-N-(3-methoxypropyl)benzamide hydrochloride;

4-{2-[3-(Morpholin-4-ylcarbonyl)phenyl]-3H-imidazo[4,5-b]pyridin-7-yl}benzamide hydrochloride;

N-(2-Methoxyethyl)-3-[7-(4-methoxyphenyl)-3H-imidazo[4,5-b]pyridin-2-yl]benzamide hydrochloride;

3-[7-(4-Methoxyphenyl)-3H-imidazo[4,5-b]pyridin-2-yl]-N-(2-pyrrolidin-1-ylethyl)benzamide hydrochloride;

N-(2-Cyanoethyl)-3-[7-(4-methoxyphenyl)-3H-imidazo[4,5-b]pyridin-2-yl]benzamide hydrochloride;

7-(4-Methoxyphenyl)-2-[3-(morpholin-4-ylcarbonyl)phenyl]-3H-imidazo[4,5-b]pyridine hydrochloride;

7-(4-Methoxyphenyl)-2-{3-[(4-methylpiperazin-1-yl)carbonyl]phenyl}-3H-imidazo[4,5-b]pyridine hydrochloride; and

3-[7-(4-Methoxyphenyl)-3H-imidazo[4,5-b]pyridin-2-yl]-N-pyridin-3-ylbenzamide hydrochloride;

as a free base or a pharmaceutically acceptable salt, solvate or solvate of a salt thereof.

The present invention also relates to compounds selected from:

2-(Benzyloxy)-4-(4-methoxyphenyl)-3-nitropyridine;

4-(4-Methoxyphenyl)-3-nitropyridin-2-amine;

4-(4-Methoxyphenyl)pyridine-2,3-diamine;

2-(Benzyloxy)-4-(3-methoxyphenyl)-3-nitropyridine;

4-(3-Methoxyphenyl)pyridine-2,3-diamine;

Methyl 4-[7-(4-methoxyphenyl)-3H-imidazo[4,5-b]pyridin-2-yl]benzoate;

Methyl 4-[7-(3-methoxyphenyl)-3H-imidazo[4,5-b]pyridin-2-yl]benzoate;

7-Chloro-2-{4-[(4-methylpiperazin-1-yl)carbonyl]phenyl}-3H-imidazo[4,5-b]pyridine;

7-Chloro-2-[4-(piperidin-1-ylcarbonyl)phenyl]-3H-imidazo[4,5-b]pyridine;

7-(4-Methoxyphenyl)-2-[4-(piperidin-1-ylcarbonyl)phenyl]-3H-imidazo[4,5-b]pyridine;

4-(7-Chloro-3H-imidazo[4,5-b]pyridin-2-yl)-N-(2-morpholin-4-ylethyl)benzamide;

Methyl 4-[7-(4-cyanophenyl)-3H-imidazo[4,5-b]pyridin-2-yl]benzoate;

7-(4-Methoxyphenyl)-2-[4-(morpholin-4-ylcarbonyl)phenyl]-3H-imidazo[4,5-b]pyridine;

4-[3-(Morpholin-4-ylcarbonyl)phenyl]-3-nitropyridin-2-amine;

4-[3-(Morpholin-4-ylcarbonyl)phenyl]pyridine-2,3-diamine;

4-[3-(Morpholin-4-ylmethyl)phenyl]pyridine-2,3-diamine;

Methyl 4-{7-[3-(morpholin-4-ylmethyl)phenyl]-1H-imidazo[4,5-b]pyridine-2-yl}benzoate;

4-{7-[3-(Morpholin-4-ylmethyl)phenyl]-1H-imidazo[4,5-b]pyridine-2-yl}benzoic acid;

Methyl 4-(7-iodo-3H-imidazo[4,5-b]pyridin-2-yl)benzoate;

Methyl 4-(7-iodo-3-{[2-(trimethylsilyl)ethoxy]methyl}-3H-imidazo[4,5-b]pyridine-2-yl)benzoate;

Methyl 4-(7-(3-{[(2-cyanoethyl)amino]carbonyl}phenyl)-3-{[2-(trimethylsilyl)ethoxy]methyl}-3H-imidazo[4,5-b]pyridin-2-yl)benzoate;

4-(7-(3-{[(2-Cyanoethyl)amino]carbonyl}phenyl)-3-{[2-(trimethylsilyl)ethoxy]methyl}3H-imidazo[4,5-b]pyridin-2-yl)benzoic acid;

N-(2-Cyanoethyl)-3-(2-[(4-methylpiperazin-1-yl)carbonyl]-3-{[2-(trimethylsilyl)ethoxy]methyl}-3H-imidazo[4,5-b]pyridin-7-yl)benzamine;

Methyl 4-(7-[3-(benzyloxy)phenyl]-3H-imidazo[4,5-b]pyridine-2-yl)benzoate;

Methyl 4-(7-[3-(benzyloxy)phenyl]-3-{[2-(trimethylsilyl)ethoxy]methyl}-3H-imidazo[4,5-b]pyridine-2-yl)benzoate;

4-(7-[3-(Benzyloxy)phenyl]-3-{[2-(trimethylsilyl)ethoxy]methyl}-3H-imdazo[4,5-b]pyridine-2-yl)benzoic acid;

7-[3-(Benzyloxy)phenyl]-2-{4-[(methylpiperazin-1-yl)carbonyl]phenyl}-3-{[2-(trimethylsilyl)ethoxy]methyl}3H-imidazo[4,5-b]pyridine;

3-(2-{4-[(4-Methylpiperazin-1-yl)carbonyl]phenyl}-3-{[2-(trimethylsilyl)ethoxy]methoxy}-3H-imidazo[4,5-b]pyridin-7-yl)phenol;

7-{3-[2-(2-Methoxyethoxy)ethoxy]phenyl}-2-{4-[(4-methylpiperazin-1-yl)carbonyl]phenyl}-3-{[2-(trimethylsilyl)ethoxy]methyl}-3H-imidazo[4,5-b]pyridine;

Methyl 4-(7-[3-(3-hydroxypropyl)phenyl]-3-{[2-(trimethylsilyl)ethoxy]methyl}-3H-imidazo[4,5-b]pyridine-2-yl)benzoate;

4-(7-[3-(3-Hydroxypropyl)phenyl]-3-{[2-(trimethylsilyl)ethoxy]methyl}-3H-imidazo[4,5-b]pyridine-2-yl)benzoic acid;

3-[3-(2-{4-Methylpiperazin-1-yl)carbonyl]phenyl}-3-{[2-(trimethylsilyl)ethoxy]methyl}-3H-imidazo[4,5-b]pyridin-7-yl)phenyl]propan-1-ol;

N-{3-[3-(2-{4-[4-Methylpiperazin-1-yl)carbonyl]phenyl}-3H-imidazo[4,5-b]pyridine-7-yl)phenoxy]propyl}acetamide;

4-[3-(2-{4-[(4-Methylpiperazin-1-yl)carbonyl]phenyl}-3-{[2-(trimethylsilyl)ethoxy]methyl}-3H-imidazo[4,5-b]pyridine-7-yl)phenoxy]butanenitrile;

3-[3-(2-{4-[(4-Methylpiperazin-1-yl)carbonyl]phenyl}-3-{[2-(trimethylsilyl)ethoxy]methyl}-3H-imidazo[4,5-b]pyridine-7-yl)phenoxy]propan-1-ol;

3-[3-(2-{4-[(4-Methylpiperazin-1-yl)carbonyl]phenyl}-3-{[2-(trimethylsilyl)ethoxy]methyl}-3H-imidazo[4,5-b]pyridine-7-yl)phenoxy]acetonitrile;

Methyl 6-[7-(4-methoxyphenyl)-3H-imidazo[4,5-b]pyridine-2-yl]nicotinate;

4-[7-(4-Methoxyphenyl)-3H-imidazo[4,5-b]pyridin-2-yl]benzoic acid;

Methyl 4-[7-(4-methoxyphenyl)-3H-imidazo[4,5-b]pyridin-2-yl]pyridine-2-carboxylate;

5-[7-(4-Methoxyphenyl)-3H-imidazo[4,5-b]pyridin-2-yl]nicotinic acid;

4-(7-iodo-3H-imidazo[4,5-b]pyridin-2-yl)benzoic acid;

7-Iodo-2-[4-(3-methoxy propyl-4-ylcarbonyl)phenyl]-3H-imidazo[4,5-b]pyridine;

7-Chloro-2-{4-[(4-methylpiperazin-1-yl)methyl]phenyl}-3H-imidazo[4,5-b]pyridine;

7-Chloro-2-[4-(morpholin-4-ylcarbonyl)phenyl]-3H-imidazo[4,5-b]pyridine;

Methyl 4-{2-[4-(morpholin-4-ylmethyl)phenyl]-3H-imidazo[4,5-b]pyridin-7-ylbenzoate;

Methyl 3-[7-(4-methoxyphenyl)-3H-imidazo[4,5-b]pyridin-2-yl]benzoate;

3-[7-(4-Methoxyphenyl)-3H-imidazo[4,5-b]pyridin-2-yl]benzoic acid;

Methyl 3-(3H-imidazo[4,5-b]pyridin-2-yl)benzoate;

[4-[2-[3-(3-methoxypropoxy)phenyl]-7-(2-trimethylsilylethoxymethyl)-5,7,9-triazabicyclo[4.3.0]nona-1,3,5,8-tetraen-8-yl]phenyl]-(4-methylpiperazin-1-yl)-methanone;

N-[3-[3-[8-[4-(4-methylpiperazin-1-yl)carbonylphenyl]-7-(2-trimethylsilylethoxymethyl)-5,7,9-triazabicyclo[4.3.0]nona-1,3,5,8-tetraen-2-yl]phenoxy]propyl]acetamide;

7-Chloro-2-[4-(morpholin-4-ylmethyl)phenyl]-3H-imidazo[4,5-b]pyridine;

Methyl 3-(7-chloro-3H-imidazo[4,5-b]pyridin-2-yl)benzoate; and

3-{7-[4-(Aminocarbonyl)phenyl]-3H-imidazo[4,5-b]pyridin-2-yl}benzoic acid.

These compounds are useful as intermediates in the process of preparing a compound according to formula I:

Listed below are definitions of various terms used in the specification and claims to describe the present invention.

In this specification the term “alkyl” includes both straight and branched chains as well as cyclic alkyl groups. The term C₁₋₃alkyl having 1 to 3 carbon atoms and may be, but is not limited to, methyl, ethyl, n-propyl, i-propyl or cyclopropyl. The term C₁₋₆alkyl having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl or cyclohexyl.

The term “C₁₋₃alkoxy” includes both straight and branched chains. The term “C₁₋₃alkoxy” having 1 to 3 carbon atoms may be, but is not limited to, methoxy, ethoxy, n-propoxy or i-propoxy.

The term “halo” or “halogen” refers to fluorine, chlorine, bromine and iodine.

The term “haloalkyl” refers to an alkyl group, defined as above, in which one or several of the hydrogen substituents have been replaced by halogen substituents, in which the term halogen is defined as above.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl”, but not limiting,are phenyl, naphthyl, indanyl or tetralinyl.

As used herein, “heteroaryl” refers to an aromatic heterocycle having at least one heteroatom ring member such as sulfur, oxygen or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7 or 5 to 6 ring-forming atoms. In some embodiments, the heteroaryl or heteroaromatic group has 1 to about 4, 1 to about 3 or 1 to 2 heteroatoms. In some embodiments, the heteroaryl or heteroaromatic group has 1 heteroatom.

The term “4-, 5-, 6- or 7-membered heterocyclic ring containing one or more heteroatoms independently selected from N, O or S” refers to a mono- or bicyclic- heterocyclic ring which may be saturated or partly saturated and which may optionally contain a carbonyl function and which may be, but is not limited to, azetidinyl, imidazolidinyl, imidazolinyl, -morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S or N, these atoms may optionally be in an oxidised form.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have sterogenic centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions is associated with glycogen synthase kinase-3 (GSK3) in mammals including man. Particularly, compounds of formula I exhibiting a selective affinity for GSK-3.

### Methods of Preparation

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Greene, P. G. M. Wuts, Wiley-Interscience, New York, 1999. It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminum trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

### Methods of Preparation of Intermediates

The processes for preparation of the intermediates, wherein R¹, R², R³, R⁴, R⁵ and A are, unless otherwise specified, as defined in formula I, comprise of the following:

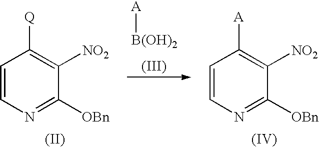

(i) Cross-coupling of a compound of formula II, wherein Q is halogen and Bn is benzyl, with a suitable aryl species III to give a compound of formula IV may be carried out by reaction with an appropriate aryl boronic acid or an aryl boronic ester. The reaction may be carried out using a suitable palladium catalyst such as Pd(PPh₃)₄, Pd(dppf)Cl₂ or Pd(OAc)₂ or Pd₂(dba)₃ together with a suitable ligand such as P(tert-butyl)3,2-(dicyclohexylphosphino)biphenyl or 2-(2′,6′-dimethoxybiphenyl)-dicyclohexylphosphine or a nickel catalyst such as nickel on charcoal or Ni(dppe)Cl₂ together with zinc and sodium triphenylphosphinetrimetasulfonate. A suitable base such as an alkyl amine, e.g. triethylamine, or potassium carbonate, sodium carbonate, cesium carbonate, sodium hydroxide or cesium fluoride may be used in the reaction, which can be performed in the temperature range of +20° C. to +160° C., using an oil bath or a microwave oven, in a suitable solvent or solvent mixture such as toluene, tetrahydrofuran, dimethoxyethane/water, N,N-dimethylformamide or dioxane. The boronic acid or boronic ester may be formed in situ, by reaction of the corresponding aryl halide (e.g., the aryl bromide) with an alkyllithium reagent such as butyllithium to form an intermediate aryl lithium species, which then is reacted with a suitable boron compound, e.g., trimethyl borate, tributyl borate or triisopropyl borate.

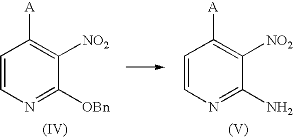

(ii) Transformation of a benzyl ether of type IV to an amine of type V can be effected by (a) first, reaction of IV with a strong organic acid, e.g. in neat trifluoroacetic acid, at a temperature in the range of 0° C. to +50° C.; (b) second, reaction of the formed intermediate with a suitable chlorinating agent such as neat phosphorus oxychloride at a temperature in the range of +80° C. to +125° C.; (c) finally, heating the resulting intermediate with ammonium hydroxide, using a microwave oven, to a temperature in the range of +100° C. to +150° C.

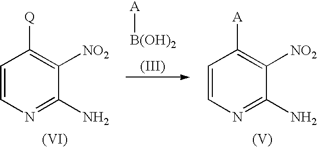

(iii) Cross-coupling of a compound of formula VI, wherein Q is halogen, with a suitable is aryl species III to give a compound of formula V can be carried out as described above for the cross-coupling of II and III to give IV.

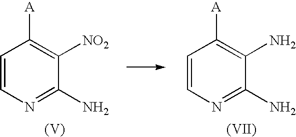

(iv) Reduction of a nitro compound of formula V to a diamine of type VII can be effected by reaction with suitable reductant, e.g. ammonium formate, in the presence of a catalyst such as palladium on charcoal, in a suitable solvent, e.g. ethanol or methanol, at a temperature in the range of +20° C. to reflux.

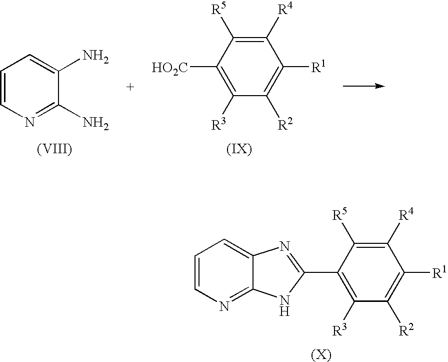

(v) Condensation of diamine VIII with a carboxylic acid of type IX to give an intermediate X can be performed by

(a) First, reacting VIII and IX in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate, in a solvent such as acetonitrile, dimethyl formamide, or a mixture thereof. A suitable base such as N,N-diisopropylethylamine may be used in the reaction, which can be performed at a temperature in the range of 0° C. to +20° C.

(b) Second, heating the resulting intermediate in a suitable organic acid, e.g. acetic acid, at a temperature in the range of +150° C. to +200° C. using an oil bath or a microwave oven.

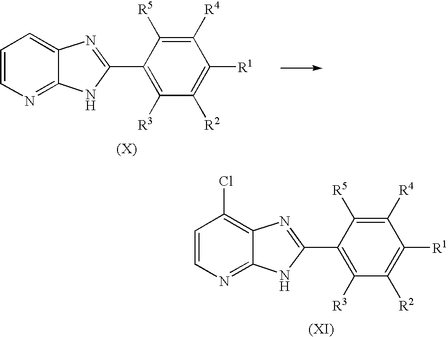

(vi) Conversion of a compound of type X into a chloride of type XI can be achieved by (a) first, reacting the compound of type X with an appropriate oxidant, e.g. m-chloroperbenzoic acid, in a suitable solvent, e.g. acetic acid, at a temperature in the range of +20° C. to +30° C.; (b) second, reaction of the formed intermediate with neat phosphorus oxychloride at a temperature in the range of +100° C. to +150° C. using an oil bath or a microwave oven.

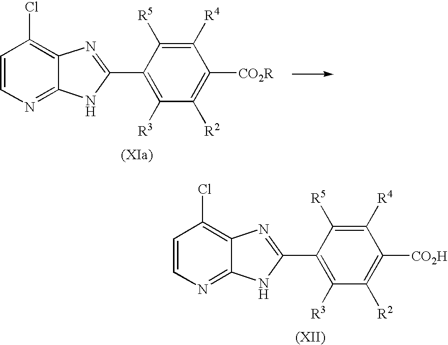

(vii) Hydrolysis of an ester of type XIa (XI, R¹ is CO₂R, wherein R is alkyl, for example, ethyl or methyl) to the corresponding acid XII might be effected by reaction with a suitable base, such as lithium, sodium or potassium hydroxide, or potassium carbonate, in mixtures of water and a suitable cosolvent, e.g. tetrahydrofuran or methanol, at a temperature in the range of +20° C. to +120° C. using an oil bath or a microwave oven.

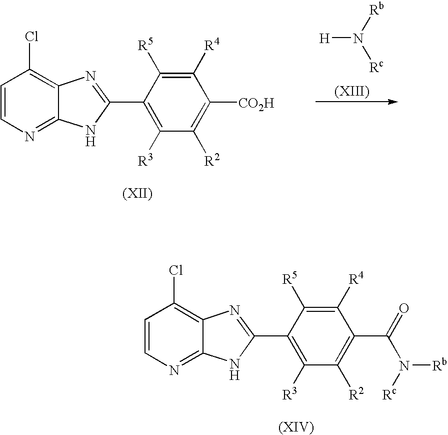

(viii) Formation of an amide of type XIV from the corresponding acid XII and an amine XIII (wherein R^{b} and R^{c} are as defined in formula I) can be performed by reacting XII and XIII in the presence of a suitable catalyst, e.g. o-benzotriazol-1-yl-N,N,N′,N′-tetramethyluroniumhexafluorophosphate or O-(7-azabenzotriazol-1-yl)-N,N,N′,N′-tetramethyluronium hexafluorophosphate in a solvent such as acetonitrile, dimethyl formamide, or a mixture thereof. A suitable base such as N,N-diisopropylethylamine may be used in the reaction, which can be performed at a temperature in the range of 0° C. to is +20° C. Alternatively, a solution of XII in a solvent such as dimethyl acetamide can be first reacted with an activating agent such as 1,1′-carbonylbis(1H-imidazole) at a temperature in the range of +80° C. to +120° C., and then reacted with the amine XIII at a temperature in the range of +100° C. to +150° C., using an oil bath or a microwave oven.

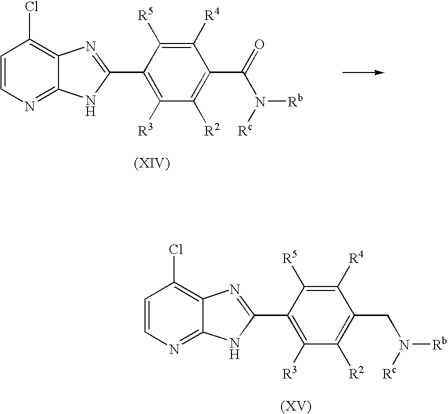

(ix) A compound of type XIV (wherein R^{b} and R^{c} are as defined in formula I) can be transformed into a compound of type XV (wherein R^{b} and R^{c} are as defined in formula I) by reaction with a suitable reducing agent, e.g. borane, in a suitable solvent such as tetrahydrofuran, at a temperature in the range of 0° C. to +60° C.

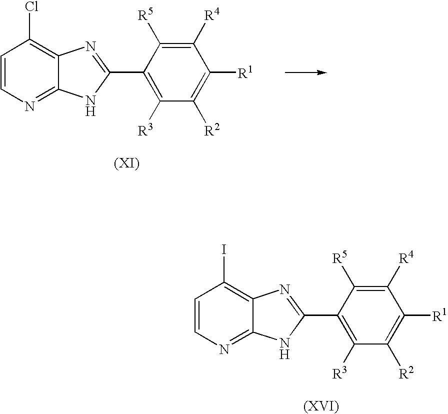

(x) A compound of type XI can be transformed into the corresponding iodide XVI by (a) first, treatment with HCl in a suitable solvent such as diethyl ether to give the hydrochloride salt, and (b) second, reaction of the salt with NaI in a suitable solvent, e.g. acetonitrile, at a temperature in the range of +150° C. to +175° C. using an oil bath or a microwave oven.

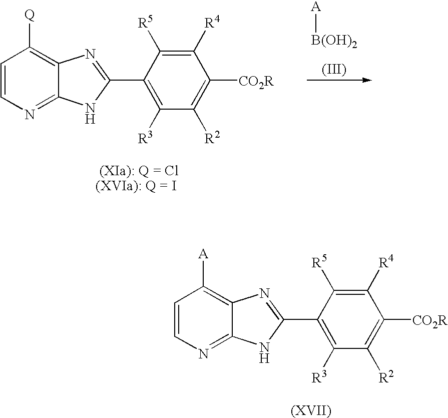

(xi) Cross-coupling of a compound of formula XIa or XVIa (XVI, wherein A is as defined above and R¹ is CO₂R wherein R is alkyl, for example methyl or ethyl) with a suitable aryl species III to give a compound of formula XVII can be carried out as described above for the cross-coupling of II and III to give IV.

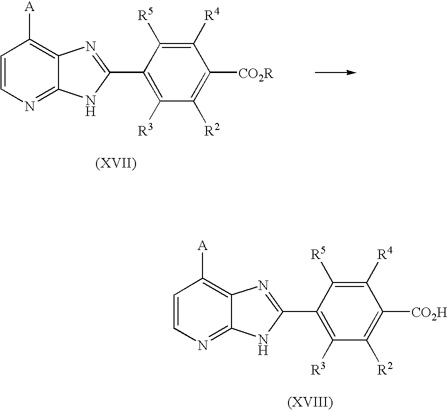

(xii) Hydrolysis of an ester of type XVII (wherein A is as defined above and R¹ is CO₂R wherein R is alkyl, for example methyl or ethyl) to the corresponding acid XVIII might be effected by reaction with a suitable base, such as lithium, sodium or potassium hydroxide, or potassium carbonate, in mixtures of water and a suitable cosolvent, e.g. tetrathydrofuran or methanol, at a temperature in the range of +20° C. to +120° C. using an oil bath or a microwave oven.

### Methods of Preparation of End Products

Another objective of the invention are processes for the preparation of a compound of general formula I, wherein R¹, R², R³, R⁴, R⁵ and A are, unless specified otherwise, defined as in formula I, comprising of:

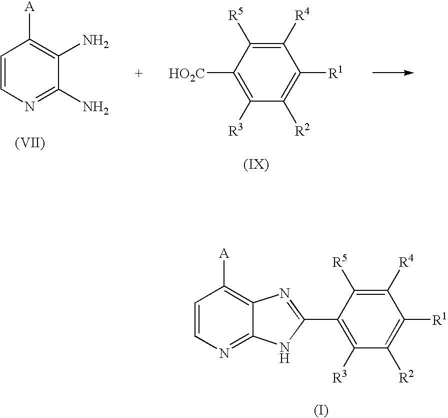

(i) Condensation of a diamine VII and a carboxylic acid of type IX to give a product of type I can be performed as described above for the preparation of X from VIII and IX.

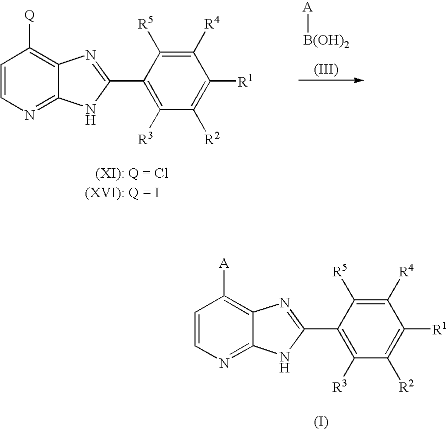

(ii) Cross-coupling of a compound of formula XI (Q=Cl) or XVI (Q=I) with a suitable aryl is species III to give a compound of formula I can be carried out as described above for the cross-coupling of II and III to give IV.

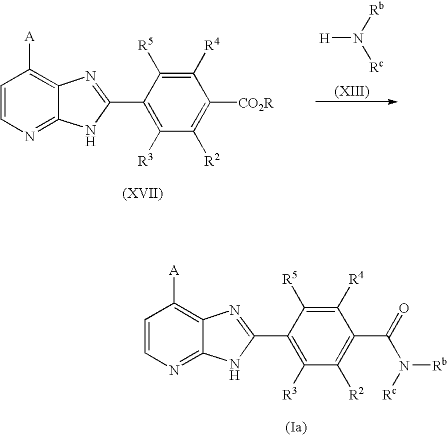

(iii) An ester of type XVII may be transformed into a compound of type Ia (I, wherein A is as defined above and wherein R^{b} and R^{c} are as defined as in formula I and wherein R¹ are CO₂R and wherein R is alkyl, for example methyl or ethyl) by (a) first, heating neat with an amine XIII at a temperature in the range of +180° C. to +220° C. using an oil bath or a microwave oven, and (b) second, after cooling, adding a suitable catalyst such as o-benzotriazol-1-yl-N,N,N′,N′-tetramethyluroniumhexafluorophosphate or O-(7-azabenzotriazol-1-yl)-N,N,′,N′-tetramethyluronium hexafluorophosphate and continuing the reaction at a temperature in the range of 0° C. to +20° C.

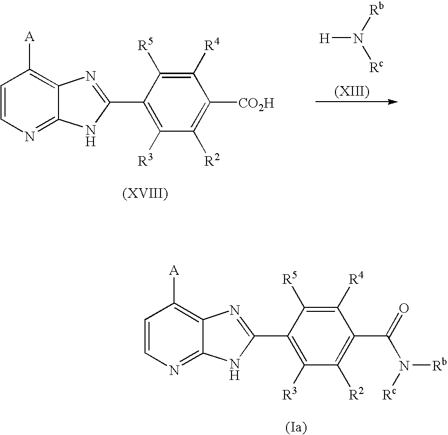

(iv) Formation of an amide of type Ia can also be performed by reacting a carboxylic acid of type XVIII (wherein R¹ is CO₂H) with an amine of type XIII (R^{b} and R^{c} are as defined as in formula I), as described for the preparation of XIV from XII and XIII.

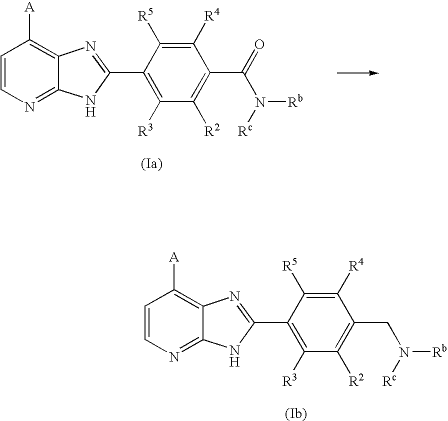

(v) A compound of type Ia can be transformed into a compound of type Ib (I, wherein A is as described above and R¹ is CH₂NR^{b}R^{c} wherein R^{b} and R^{c} are as defined as in formula I) by reduction, as described for the transformation of XIV to XV.

Consequently, in one aspect of the present invention, there is provided a process for preparing a compound of formula I, wherein R¹, R², R³, R⁴, R⁵ and A are, unless specified otherwise, defined as in formula I, comprising of:

(i) Condensation of a diamine VII and a carboxylic acid of type IX by first reacting the components in the presence of a suitable catalyst, optionally with an added base, and then heating the resulting intermediate in a suitable organic acid.

(ii) Cross-coupling of a compound of formula XI (Q=Cl) or XVI (Q=I) with a suitable aryl species III by reaction in the presence of a suitable metal catalyst, optionally with an added organic or inorganic base.

(iii) An ester of type XVII may be coupled with an amine XIII to give a compound of type Ia (I, R¹═C(O)NR^{b}R^{c}, wherein R^{b} and R^{c} are as defined as in formula I) by first heating XVII with the neat amine XIII, and then adding a suitable catalyst and continuing the reaction.

(iv) Formation of an amide of type Ia can also be performed by reacting a carboxylic acid of type XVIII with an amine of type XIII, in the presence of a suitable catalyst, optionally with an added amine base. Alternatively, the acid XVIII can be first reacted with an activating agent, and then reacted with the amine.

(v) A compound of type Ia can be transformed into a compound of type Ib (I, A is as defined above and R¹ is C—CH₂NR^{b}R^{c}, wherein R^{b} and R^{c} are as defined as in formula I) by treatment with a suitable reducing agent.

The hydrochloric salt of a compound of formula I may be obtained from a compound of formula I by treatment with hydrochloric acid at a temperature in the range of 0° C. to +25° C., in a suitable solvent such as dichloromethane, tetrahydrofuran or a dichloromethane/methanol mixture.

### General Methods

All solvents used were analytical grade and commercially available anhydrous solvents were routinely used for reactions. Reactions were typically run under an inert atmosphere of nitrogen or argon.

¹H and ¹³C NMR spectra were recorded at 400 MHz for proton, 376 MHz for fluorine-19 and 100 MHz for carbon-13, either on a Varian Unity+400 NMR Spectrometer equipped with a 5 mm BBO probehead with Z-gradients, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton and 100 MHz for carbon-13. The following reference signals were used: the middle line of DMSO-d₆ δ 2.50 (¹H), δ 39.51 (¹³C); the middle line of CD₃OD δ 3.31 (¹H) or δ 49.15 (¹³C), CDCl₃ 6 7.26 (¹H) and the middle line of CDCl₃ δ 77.16 (¹³C) (unless otherwise indicated).

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A:95:5 0.1M NH₄OAc:MeCN or 95:5 8 mM HCOOH:MeCN) ending at 100% B (MeCN).

Alternatively, mass spectra were recorded on a Waters LC-MS system (Sample Manager 2777C, 1525μ binary pump, 1500 Column Oven, ZQ, PDA2996 and ELS detector, Sedex 85). Separation was performed using a Zorbax column (C8, 3.0×50 mm, 3 μm). A four minutes linear gradient was used starting at 100% A (A: 95:5 10 mM NH₄OAc:MeOH ) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode.

Microwave heating was performed in a Creator or Smith Synthesizer Single-mode microwave cavity producing continuous irradiation at 2450 MHz.

HPLC analyses were performed on an Agilent BPP1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (95:5 10 mM NH₄OAc:MeCN) and ending at 100% B (B: acetonitrile), in 4 min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns. Typical solvents used for flash chromatography was mixtures of heptane/ethyl acetate. SCX ion exchange columns were performed on Isolute® columns. Chromatography through ion exchange columns were typically performed in solvents or solvent mixtures such a methanol and 10% ammonia in methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (Cl8, 5 μm, 100 mm×19 mm). Narrow gradients with MeCN/0.1% trifluoroacetic acid in MilliQ Water were used at a flow rate of 10 ml/min.

Alternatively preparative chromatography was run on a Waters FractionLynx system with a Autosampler combined Automated Fraction Collector (Waters 2767), Gradient Pump (Waters 2525), Regeneration Pump (Waters 600), Make Up Pump (Waters 515), Waters Active Splitter, Column Switch (Waters CFO), PDA (Waters 2996) and Waters ZQ mass spectrometer. Column; XBridge™ Prep C8 5 μm OBDT 19×100 mm, with guard column; XTerra® Prep MS C8 10 μm 19×10 mm Cartridge. A gradient from 100% A (95% 0.1M NH₄OAc in MilliQ water and 5% MeCN) to 100% B (100% MeCN) was applied for LC-separation at flow rate 25 ml/min. The PDA was scanned from 210-350 nm. The ZQ mass spectrometer was run with ESI in positive mode. The Capillary Voltage was 3 kV and the Cone Voltage was 30V. Mixed triggering, UV and MS signal, determined the fraction collection.

The formation of hydrochloride salts of the final products were typically performed by dissolution in solvents or solvent mixtures such as diethyl ether, tetrahydrofuran, dichloromethane/methanol, followed by addition of 1M HCl in diethyl ether.

The following abbreviations have been used:
aq. aqueous;
CDI carbonyl diimidazole;
CH₂Cl₂ dichloromethane;
is DIPEA N-N-diisopropylethylamine;
DMF N-N-dimethylformamide;
ether diethyl ether;
EtOAc ethyl acetate;
EtOH ethanol;
HBTU o-benzotriazol-1-yl-N,N,N′,N′-tetramethyluronium
hexafluorophosphate;
HCl hydrochloride;
HOAc acetic acid;
(i-Pr)₂EtN N-N-diisopropylethylamine;
m-CPBA 3-chloroperoxybenzoic acid;
MeCN acetonitrile;
MeOH methanol;
NaHCO₃ sodium hydrogen carbonate;
Na₂SO₄ sodium sulphate;
NH₃ ammonia;
NH₄OAc ammonium acetate;
Pd(OAc)₂ palladium diacetate;
PdCl₂(dppf)*DCM (1,1′-bis(diphenylphosphino)ferrocen)palladium(II) chloride dichloromethane adduct;
Pd₂(dba)₃ tris(dibenzylideneacetone)dipalladium;
Pd(dppf)Cl₂ (1,1′-bis(diphenylphosphino)ferrocen)palladium(II) chloride;
Pd(PPh₃)₄ tris(tri-phenylphosphine)palladium;
Ni(dppe)Cl₂ (1,2-bis(diphenylphosphino)ethane)nickel(II) chloride;
POCl₃ trichlorophosphorous oxide;
SEM-Cl 2-trimethylsilylethoxymethyl chloride;
RT retention time (on HPLC or LCMS);
r.t. room temperature;
THF tetrahydrofuran;
TSTU o-(N-succinimidyl)-N,N,N′,N′-tetramethyluronium tetrafluoroborate.

Starting materials used were either available from commercial sources or prepared according to literature procedures and had experimental data in accordance with those reported. The following is an example of a starting material that was prepared: 2-(Benzyloxy)-4-chloro-3-nitropyridine: Arvanitis, A. G., et al, Bioorganic & Medicinal Chemistry Letters, 2003, 13, 125-128.

Compounds have been named either using ACD/Name, version 8.08, software from Advanced Chemistry Development, Inc. (ACD/Labs), Toronto ON, Canada, www.acdlabs.com, 2004 or using Openeye lexichem version 1.4 (Copyright© 1997-2006 OpenEye Scientific Software, Santa Fe, N.Mex.) to generate the IUPAC name.

In the following general methods A to E, the groups R¹, R² and R³ are used independantly to indicate the diversity of substitution within each structure. The identity of R¹, R² and R³ will be clear to a person skilled in the art based on the starting materials and intermediates for each specific example. For instance in Example 73, which refers to General method E, E1 is 3-[7-(4-methoxyphenyl)-3H-imidazo[4,5-b]pyridin-2-yl]benzoic acid such that R¹ is 7-(4-methoxyphenyl)- and E2 is 3-aminopropionitrile such that R² is hydrogen and R³ is —CH₂CH₂CN.

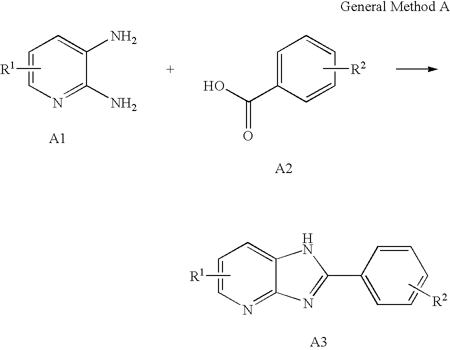

DIPEA or triethylamine (3.0 equiv.) was added to a suspension of the diamine A1 (1.0 equiv.), the benzoic acid A2 (1.1 equiv.) and HBTU (1.1 equiv.) in DMF, and the reaction mixture was stirred at room temperature for 30 minutes. The solvent was removed in vacuo and the residue was mixed with HOAc and heated in a microwave reactor at +180° C. for 10 minutes. The product, which precipitated at room temperature, was collected by filtration, washed with water, dried, and used in the next step without further purification.

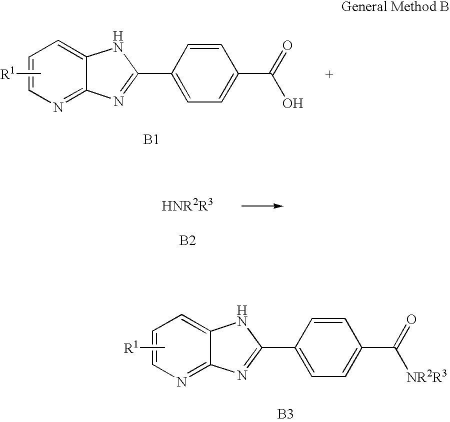

DIPEA (3.0 equiv.) was added to a suspension of the benzoic acid B1 (1.0 equiv.), the amine B2 (1.2 equiv.) and HBTU or TSTU (1.2 equiv.) in MeCN or DMF (5 mL) and the reaction mixture was stirred at room temperature for 30 minutes. Saturated NaHCO₃ (aq.) was added and the precipitated product was collected by filtration, washed with water and dried. The product was used in the next step without further purification.

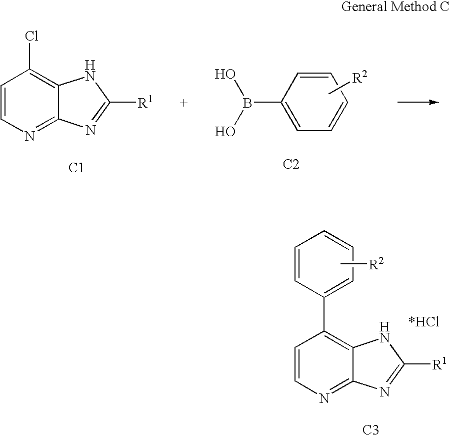

A mixture of the imidazopyridine C1 (1.0 equiv.), the boronic acid C2 (2.0 equiv.), PdCl₂(dppf)*DCM (0.05 equiv.) and sodium carbonate (4.0 equiv.) in THF/water (9:1) were heated in a microwave reactor at +140° C. for 10 minutes. After cooling to room temperature, the mixture was diluted with EtOAc and washed with water. The organic phase was dried (Na₂SO₄), filtered and evaporated in vacuo. The residue was purified by preparative HPLC, which afforded the product as a base. The base was dissolved in CH₂Cl₂/MeOH (9:1) and hydrochloric acid (1M HCl in diethyl ether) was added until precipitation formed. The solid hydrochloride salt was collected by filtration and dried.

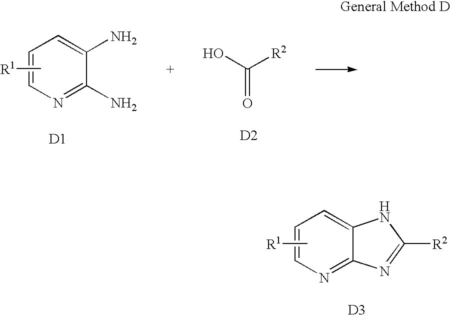

POCl₃ 2 mL (solvent) was added to a mixture of the diamine D1 (1.0 equiv.), the carboxylic acid D2 (1.1 equiv.) and the reaction mixture was heated in a microwave reactor at +160° C. for 20 minutes. The product mixture was mixed with ice/water mixture. The products were collected by filtration, or by extraction with CH₂Cl₂. Purification by preparative HPLC, afforded each product as a base. The base was dissolved in CH₂Cl₂/MeOH (9:1) and hydrochloric acid (1M HCl in diethyl ether) was added until precipitation formed. The solid hydrochloride salt was collected by filtration and dried.

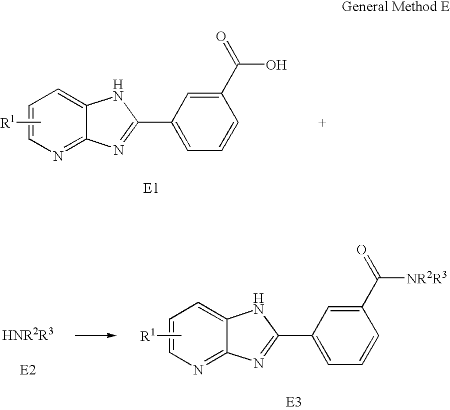

Et₃N (3.0 equiv.) was added to a suspension of the benzoic acid E1 (1.0 equiv.), and TSTU (1.2 equiv.) in DMF (5 mL) and the reaction mixture was stirred at room temperature for 30 minutes. The amine E2 (1.2 equiv.) was added and the reaction mixture was stirred for 1 h. The residue was purified by preparative HPLC, which afforded the product as a base. The base was dissolved in CH₂Cl₂/MeOH (9:1) and hydrochloric acid (1M HCl in diethyl ether) was added until precipitation formed. The solid hydrochloride salt was collected by filtration and dried.

### WORKING EXAMPLES

Below follows a number of non-limiting examples of the compounds of the present invention.

### Example 1

### 7-(4-Methoxyphenyl)-2-{4-[(4-methylpiperazin-1-yl)sulfonyl]phenyl}-3H-imidazo[4,5-b]pyridine hydrochloride

### Example 1(a)

### 2-(Benzyloxy)-4-(4-methoxyphenyl)-3-nitropyridine

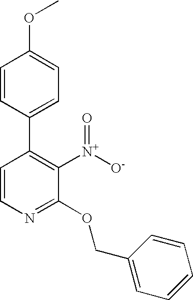

A suspension of 2-(benzyloxy)-4-chloro-3-nitropyridine (Arvanitis, A. G., et al, Bioorganic & Medicinal Chemistry Letters, 2003, 13, 125-128) (0.5 g, 1.89 mmol), (4-methoxyphenyl)boronic acid (0.345 g, 227 mmol), PdCl₂(dppf)*DCM (0.093 g, 0.114 mmol) and sodium carbonate (0.723 g, 6.82 mmol) in THF/water 9:1 (10 mL) was heated under reflux for 1 h. The mixture was allowed to cool to r.t., and was diluted with EtOAc (100 mL) and washed with water (50 mL) and brine (50 mL). The organic phase was dried (Na₂SO₄) and evaporated. Purification by flash chromatography (heptane/EtOAc-gradient; 0-30% EtOAc) afforded 0.553 g (55%) of the title compound as a solid.

¹H NMR (CDCl₃) δ ppm 8.24 (d, J=5.6 Hz, 1 H), 7.47-7.43 (m, 2 H), 7.42-7.30 (m, 5 H), 7.02-6.91 (m, 3 H), 5.55 (s, 2 H), 3.85 (s, 3 H).

### Example 1(b)

### 4-(4-Methoxyphenyl)-3-nitropyridin-2-amine

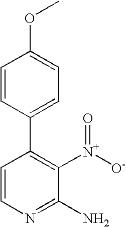

2-(Benzyloxy)-4-(4-methoxyphenyl)-3-nitropyridine obtained from Example 1(a) (0.5 g, 1.49 mmol) was stirred in trifluoroacetic acid (5 mL), at r.t. for 5 h. The solvent was evaporated and the residue was washed with 20% EtOAc/hexane mixture and dried. The solid was heated at +100° C. for 8 h in POCl₃ (5 mL). The mixture was cooled and then poured into ice/water and extracted with EtOAc (2×100 mL). The organic phase was washed with saturated NaHCO₃ (aq.), brine, dried (Na₂SO₄) and evaporated in vacuo to afford 0.326 g (83%) crude of the intermediate product; 2-chloro-4-(4-methoxyphenyl)-3-nitropyridine. The intermediate product, 2-chloro-4-(4-methoxyphenyl)-3-nitropyridine (0.3 g, 1.14 mmol), was heated with ammonium hydroxide (4 mL) in a microwave reactor at +130° C. for 2000 s. The mixture was diluted with water (50 mL) and the precipitate was filtered, washed with water and dried, affording the title compound in 0.233 g (84%) yield. The crude product was used in the next step without further purification.

¹H NMR (CDCl₃) δ ppm 8.12 (d, J=5.1 Hz, 1 H), 7.18 (d, J=9.1 Hz, 2 H), 6.88 (d, J=8.6 Hz, 2 H), 6.59 (d, J=5.1 Hz, 1 H), 5.79 (s, 2 H), 3.77 (s, 3 H).

### Example 1(c)

### 4-(4-Methoxyphenyl)pyridine-2,3-diamine

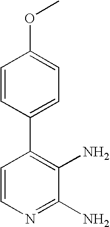

Palladium on charcoal (0.015 g, 0.14 mmol) was added to a mixture of 4-(4-methoxyphenyl)-3-nitropyridin-2-amine (0.230 g, 0.939 mmol), which was obtained from Example 1(b), and ammonium formate (0.237 g, 3.76 mmol) in EtOH (50 mL). The mixture was heated to reflux for 1 h. The mixture was cooled and filtered through diatomaceous earth and the solvent was evaporated in vacuo. Purification on a SCX ion exchange column (10% NH₃/MeOH) afforded 0.179 g (89%) of the title compound.

MS (ESI) m/z 216 (M+1).

### Example 1(d)

### 7-(4-Methoxyphenyl)-2-(4-[(4-methylpiperazin-1-yl)sulfonyl]phenyl}-3H-imidazo[4,5-b]pyridine hydrochloride

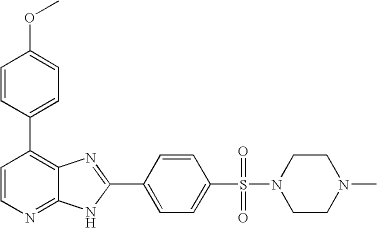

DIPEA (0.284 mL, 1.63 mmol) was added to a suspension of 4-(4-methoxyphenyl)pyridine-2,3-diamine (0.117 g, 0.543 mmol), which was obtained from Example 1(c), and 4-[(4-methylpiperazine-1-yl)sulfonyl]benzoic acid (0.154 g, 0.543 mmol) and HBTU (0.247 g, 0.651 mmol) in DMF, and stirred at r.t. for 1 h. Saturated NaHCO₃ (aq.) was added to the reaction mixture and a precipitate was formed. The precipitate was filtered, washed with water and dried. The solid was mixed with HOAc (4 mL) and heated in a microwave reactor at +120° C. for 600 s. The solvent was removed in vacuo, and the residue was purified by preparative HPLC to afford 0.025 g of the product as a base. The hydrochloride salt was prepared by dissolving the base in CH₂Cl₂/MeOH (2 mL, 9:1), 1M HCl in ether (2 mL) was added and the precipitated was collected by filtration and dried, affording 0.028 g (9%) of the title compound.

¹H NMR (CD₃OD) δ ppm 8.59 (d, J=8.6 Hz, 2 H), 8.55 (d, J=6.1 Hz, 1 H), 8.13-8.04 (m, 4 H), 7.74 (d, J=6.1 Hz, 1 H), 7.23 (d, J=9.1 Hz, 2 H), 4.10-3.96 (m, 2 H), 3.95 (s, 3 H), 3.68-3.53 (m, 2 H), 2.92 (s, 3 H), 2.87-2.77 (m, 2 H);

MS (ESI) m/z 464 (M+1).

### Example 2

### 7-(3-Methoxyphenyl)-2-{4-[(4-methylpiperazin-1-yl)sulfonyl]phenyl}-3H-imidazo[4,5-b]pyridine hydrochloride

### Example 2(a)

### 2-(Benzyloxy)-4-(3-methoxyphenyl)-3-nitropyridine

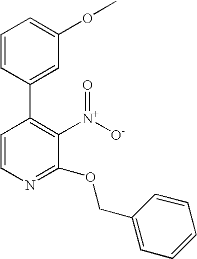

The title compound was prepared in accordance with the general method of Example 1(a) using 2-(benzyloxy)-4-chloro-3-nitropyridine (0.5 g, 1.89 mmol) and (3-methoxyphenyl)boronic acid (0.345 g, 227 mmol), affording the title compound in 0.512 g (80%) yield.

¹H NMR (CDCl₃) δ ppm 8.27 (d, J=5.1 Hz, 1 H), 7.48-7.42 (m, 2 H), 7.41-7.31 (m, 4 H), 7.03-6.95 (m, 3 H), 6.95-6.90 (m, 1 H), 5.56 (s, 2 H), 3.81 (d, 3 H).

### Example 2(b)

### 4-(3-Methoxyphenyl)pyridine-2,3-diamine

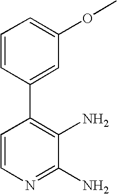

2-(Benzyloxy)-4-(3-methoxyphenyl)-3-nitropyridine (0.512 g, 1.52 mmol) obtained from Example 2(a) was stirred in trifluoroacetic acid (5 mL), at r.t. for 5 h. The solvent was evaporated and the residue was washed with 20% EtOAc/hexane mixture and dried. The solid was heated at +100° C. for 8 h in POCl₃ (5 mL). After cooling the mixture was poured into ice/water and extracted with EtOAc (2×100 mL). The organic phase was washed with saturated NaHCO₃ (aq.), brine, dried (Na₂SO₄) and evaporated in vacuo to afford 0.326 g (83%) crude of the intermediate product; 2-chloro-4-(3-methoxyphenyl)-3-nitropyridine. The intermediate product, 2-chloro-4-(3-methoxyphenyl)-3-nitropyridine (0.3 g, 1.14 mmol), was heated with ammonium hydroxide (4 mL) in a microwave reactor at +130° C. for 2000 s. The mixture was diluted with water (50 mL) and the precipitate was filtered, washed with water and dried to afford 0.233 g (84%) of the second intermediate product; 4-(3-methoxyphenyl)-3-nitropyridine-2-amine. Palladium on charcoal (0,015 g, 0.14 mmol) was added to a mixture of 4-(3-methoxyphenyl)-3-nitropyridine-2-amine (0.150 g, 0.612 mmol) and ammonium formate (0.237 g, 3.76 mmol) in EtOH (50 mL). The mixture was heated to reflux for 1 h. The mixture was cooled and filtered through diatomaceous earth and the solvent was evaporated in vacuo. Purification on a SCX ion exchange column (10% NH₃/MeOH) afforded 0.121 g (92%) of the title compound.

MS (ESI) m/z 216 (M+1).

### Example 2(c)

### 7-(3-Methoxyphenyl)-2-[4-[(4-methylpiperazin-1-yl)sulfonyl]phenyl}-3H-imidazo[4,5-b]pyridine hydrochloride

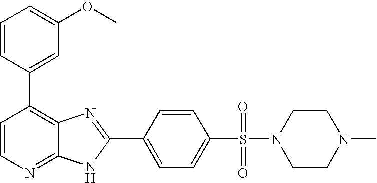

The title compound was prepared in accordance with the general method of Example 1(d) using 4-(3-methoxyphenyl)pyridine-2,3-diamine (0.055 g, 0.256 mmol) obtained from Example 2b and 4-[(4-methylpiperazine-1-yl)sulfonyl]benzoic acid (0.080 g, 0.281 mmol), affording 0.028 g (67%) of the title compound.

¹H NMR (CD₃OD) δ ppm 8.67 (d, J=6.1 Hz, 1 H), 8.60 (d, J=8.6 Hz, 2 H), 8.08 (d, J=9.1 Hz, 2 H), 7.86 (d, J=6.1 Hz, 1 H), 7.66-7.59 (m, 1 H), 7.54-7.47 (m, 2 H), 7.33-7.23 (m, J=8.1, 2.5 Hz, 1 H), 4.07-3.99 (m, 2 H), 3.94 (s, 3 H), 3.64-3.55 (m, 2 H), 3.28-3.20 (m, 2 H), 2.91 (s, 3 H), 2.89-2.83 (m, 2 H); MS (ESI) m/z 464 (M+1).

### Example 3

### 7-(4-Methoxyphenyl)-2-{4-[(4-methylpiperazin-1-yl)carbonyl]phenyl}-3H-imidazo[4,5-b]pyridine hydrochloride

### Example 3(a)

### Methyl 4-[7-(4-methoxyphenyl)-3H-imidazo[4,5-b]pyridin-2-yl]benzoate

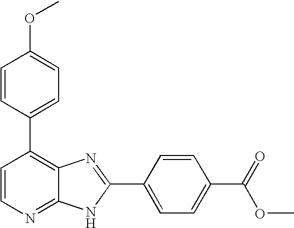

The title compound was prepared in accordance with the general method A using 4-(4-methoxyphenyl)pyridine-2,3-diamine (obtained from Example 1(c)) (55 mg, 0.256 mmol) and terephtalic acid monomethyl ester (51 mg, 0.281 mmol), affording a crude yield of 33 mg (36%), and was used without purification in the next step.

MS (ESI) m/z 360 (M+1).

### Example 3(b)

### 7-(4-Methoxyphenyl)-2-(4-[(4-methylpiperazin-1-yl)carbonyl]phenyl}-3H-imidazo[4,5-b]pyridine hydrochloride

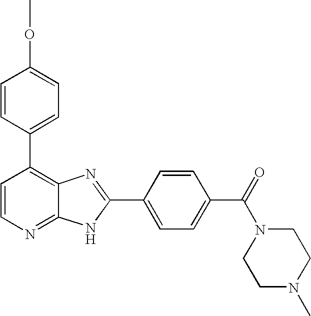

Methyl 4-[7-(4-methoxyphenyl)-3H-imidazo[4,5-b]pyridin-2-yl]benzoate (0.025 g, 0.0698 mmol) obtained from Example 3(a) and N-methylpiperazine (2 mL) was heated in microwave reactor at +200° C. for 12 minutes. After cooling to r.t., HBTU (0.032 g, 0.0835 mmol) was added and the reaction mixture was stirred at r.t. for 16 h. The solvent was evaporated and the residue was purified by preparative HPLC. The hydrochloride salt was prepared by dissolving the base in CH₂Cl₂/MeOH (2 mL, 9:1), 1M HCl in ether (2 mL) was added to the mixture and the precipitated was collected by filtration and dried, affording 0.019 g (55%) of the title compound.

¹H NMR (CD₃OD) δ ppm 8.58 (d, J=6.3 Hz, 1 H), 8.48 (d, J=8.3 Hz, 2 H), 8.04-7.93 (m, 2 H), 7.80 (d, J=6.3 Hz, 1 H), 7.77 (d, J=8.3 Hz, 2 H), 7.30-7.23 (m, 2 H), 3.96 (s, 3 H), 3.74-3.40 (m, 4 H), 3.30-3.11 (m, 2 H), 2.97 (s, 3 H); MS (APPI) m/z 428 (M+1).

### Example 4

### 7-(3-methoxyphenyl)-2-{4-[(4-methylpiperazin-1-yl)carbonyl]phenyl}-3H-imidazo[4,5-b]pyridine hydrochloride

### Example 4(a)

### Methyl 4-[7-(3-methoxyphenyl)-3H-imidazo[4,5-b]pyridin-2-yl]benzoate

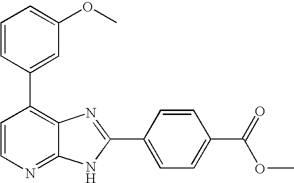

The title compound was prepared in accordance with the general method A using 4-(3-methoxyphenyl)pyridine-2,3-diamine (obtained from Example 2(b)) (51 mg, 0.281 mmol) and terephtalic acid monomethyl ester (51 mg, 0.281 mmol), affording a crude yield of 37 mg (40%), and was used without purification in the next step.

MS (ESI) m/z 360 (M+1).

### Example 4(b)

### 7-(3-Methoxyphenyl)-2-{4-[(4-methylpiperazin-1-yl)carbonyl]phenyl}-3H-imidazo[4,5-b]pyridine hydrochloride

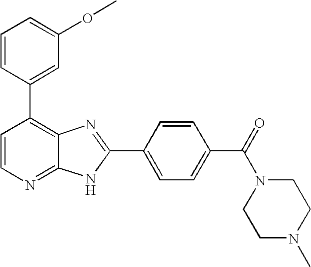

The title compound was prepared in accordance with the general method of Example 3(b) using methyl 4-[7-(3-methoxyphenyl)-3H-imidazo[4,5-b]pyridin-2-yl]benzoate (25 mg, 0.07 mmol) obtained from Example 4(a), affording 21 mg (60%) of the title compound.

¹H NMR (CD₃OD) δ ppm 8.64 (d, J=6.1 Hz, 1 H), 8.46 (d, J=8.6 Hz, 2 H), 7.83 (d, J=6.1 Hz, 1 H), 7.76 (d, J=8.6 Hz, 2 H), 7.68-7.58 (m, 1 H), 7.54-7.45 (m, 2 H), 7.32-7.20 (m, 1 H), 3.94 (s, 3 H), 3.74-3.44 (m, 4 H), 3.28-3.15 (m, 4 H), 2.97 (s, 3 H); MS (APPI) m/z 428 (M+1).

### Example 5

### 7-(4-Chlorophenyl)-2-{4-[(4-methylpiperazin-1-yl)carbonyl]phenyl}-3H-imidazo[4,5-b]pyridine hydrochloride

### Example 5(a)

### Methyl 4-(3H-imidazo[4,5-b]pyridin-2-yl)benzoate

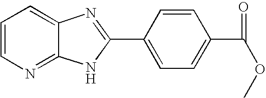

DIPEA (24 mL, 138 mmol) was added to a suspension of pyridine-2,3-diamine (5.0 g, 45.9 mmol), terephtalic acid monomethyl ester (8.26 g, 45.9 mmol) and HBTU (20.9 g, 55.0 mmol) in MeCN (200 mL) and the reaction mixture was stirred at r.t. for 1 h. A precipitate that formed was collected and washed with MeCN. The solid was distributed into microwave vials with HOAc (4 mL) and heated to +200° C. for 5 minutes. The product precipitated at r.t. and was filtered, washed with HOAc and MeCN and dried to afford 9.6 g (83% yield) of the title compound.

MS (ESI) m/z 254 (M+1).

### Example 5(b)

### Methyl 4-(7-chloro-3H-imidazo[4,5-b]pyridin-2-yl)benzoate

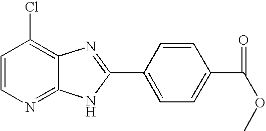

Methyl 4-(3H-imidazo[4,5-h]pyridin-2-yl)benzoate (8.3 g, 32.8 mmol), which was is obtained from Example 5(a) and m-CPBA (70%, 22 g, 98.4 mmol) in HOAc was stirred at r.t. for 18 h. The solvent was evaporated in vacuo and the residue was crystallized from EtOH. The solid was mixed with POCl₃ and heated in a microwave reactor at +120° C. for 5 minutes. After cooling to r.t., the mixture was poured into ice/water mixture and the precipitate that formed was collected, washed with water and dried, affording the title compound in 8.0 g (85%) yield.

¹H NMR (DMSO-d₆) δ ppm 8.46-8.39 (m, 2 H), 8.34 (d, J=5.3 Hz, 1 H), 8.17-8.10 (m, 2 H), 7.46 (d, J=5.3 Hz, 1 H), 3.90 (d, 3 H); MS (ESI) m/z 288 (M+1).

### Example 5(c)

### 4-(7-Chloro-3H-imidazo[4,5-b]pyridin-2-yl)benzoic acid

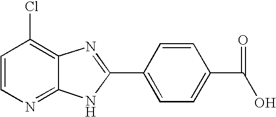

A mixture of methyl 4-(7-chloro-3H-imidazo[4,5-b]pyridin-2-yl)benzoate (7.7 g, 26.8 mmol), which was obtained from Example 5(b), and lithium hydroxide (6.0 g, 250 mmol) in THF/water (9:1) was heated in microwave reactor at +120° C. for 10 minutes. After cooling to r.t., the mixture was made neutral by using 2M HCl (aq.). The precipitate was s filtered, washed with water and dried to afford the title compound in 7.0 g (96%) yield.

¹H NMR (DMSO-d₆) δ ppm 8.28 (d, J=8.3 Hz, 2 H), 8.23 (d, J=5.3 Hz, 1 H), 8.07 (d, J=8.1 Hz, 2 H), 7.34 (d, J=5.3 Hz, 1 H); MS (APPI) m/z 274 (M+1).

### Example 5(d)

### 7-Chloro-2-{4-[(4-methylpiperazin-1-yl)carhonyl]phenyl}-3H-imidazo[4,5-b]pyridine

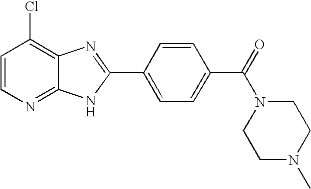

The title compound was prepared in accordance with the general method B using 4-(7-chloro-3H-imidazo[4,5-b]pyridin-2-yl)benzoic acid (1.0 g, 3.66 mmol), obtained from Example 5(c) and N-methylpiperazine (0.44 g, 4.40 mmol), affording a crude yield of 1.1 g (91%).

MS (APPI) m/z 356 (M+1).

### Example 5(e)

### 7-(4-chlorophenyl)-2-{4-[(4-methylpiperazin-1-yl)carbonyl]phenyl}-3H-imidazo[4, 5-b]pyridine hydrochloride

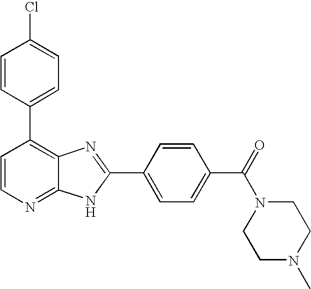

The title compound was prepared in accordance with the general method C using 7-chloro-2-{4-[(4-methylpiperazin-1-yl)carbonyl]phenyl}-3H-imidazo[4,5-b]pyridine (0.200 g, 0.563 mmol), obtained from Example 5(d) and (4-chlorophenyl)boronic acid (0.176 g, 1.13 mmol), affording 0.065 g (23%) of the title compound.

¹H NMR (DMSO-d₆) δ ppm 8.48 (d, J=5.3 Hz, 1 H), 8.41 (d, J=8.3 Hz, 2 H), 8.37 (d, J=8.3 Hz, 2 H), 7.72-7.66 (m, 4 H), 7.64 (d, J=5.3 Hz, 1 H), 4.57 (s, 3 H), 3.22-3.00 (m, 4 H), 2.79 (s, 3 H); MS (APPI) m/z 432 (M+1).

### Example 6

### 7-(4-Methoxyphenyl)-2-[4-(piperidin-1-ylmethyl)phenyl]-3H-imidazo[4,5-b]pyridine hydrochloride

### Example 6(a)

### 7-Chloro-2-[4-(piperidin-1-ylcarbonyl)phenyl]-3H-imidazo[4,5-b]pyridine

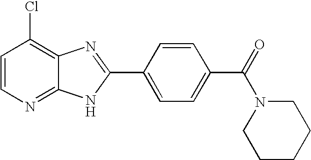

The title compound was prepared in accordance with the general method B using 4-(7-chloro-3H-imidazo[4,5-b]pyridin-2-yl)benzoic acid (obtained from Example 5(c)) (0.876 g, 3.21 mmol) and piperidine (0.327 g, 3.85 mmol), affording a crude yield of 0.829 g (76%).

MS (APPI) m/z 341 (M+1).

### Example 6(b)

### 7-(4-Methoxyphenyl)-2-[4-(piperidin-1-ylcarboyzyl)phenyl]-3H-imidazo[4, 5-b]pyridine

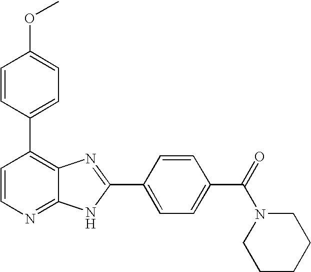

The title compound was prepared in accordance with the general method C, with the exception that the base was obtained. Using 7-chloro-2-[4-(piperidin-1-ylcarbonyl)phenyl]-3H-imidazo[4,5-b]pyridine (62 mg, 0.182 mmol), which was obtained from Example 6(a), (4-methoxyphenyl)boronic acid (69 mg, 0.454 mmol), PdCl₂(dppf)*DCM (9.3 mg, 0.011 mmol) and sodium carbonate (72 mg, 0.68 mmol), the title compound was obtained in 35 mg (39%) yield.

MS (APPI) m/z 413 (M+1).

### Example 6(c)

### 7-(4-Methoxyphenyl)-2-[4-(piperidin-1-ylmethyl)phenyl]-3H-imidazo[4,5-b]pyridine hydrochloride

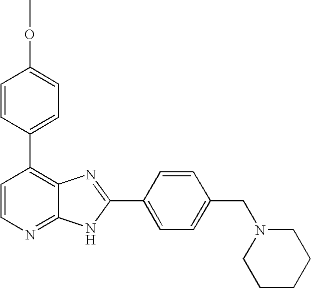

Borane-THF complex (1M, 1 mL) was added to 7-(4-methoxyphenyl)-2-[4-(piperidin-1-ylcarbonyl)phenyl]-3H-imidazo[4,5-b]pyridine (0.030 g, 0.073 mmol), obtained from Example 6(b) at r.t.. After stirring at r.t. for 45 minutes, MeOH (1 mL) was added dropwise to the reaction mixture and the mixture was stirred for 2 h at r.t. The solvent was evaporated in vacuo, and the crude product was purified by preparative HPLC. The hydrochloride salt was prepared by dissolving the base in CH₂Cl₂/MeOH (2 mL, 9:1), 1M HCl in ether (2 mL) was added and the precipitated was collected by filtration and dried, affording 19 mg (56%) of the title compound.

¹H NMR (CD₃OD) δ ppm 8.57 (d, J=5.3 Hz, 1 H), 8.47 (d, J=7.3 Hz, 2 H), 7.99 (d, J=8.1 Hz, 2 H), 7.86-7.72 (m, 3 H), 7.25 (d, J=8.1 Hz, 2 H), 4.44 (s, 2 H), 3.95 (s, 3 H), 3.56-3.43 (m, 3 H), 3.13-2.97 (m, 2 H), 2.01-1.92 (m, 2 H), 1.91-1.74 (m, 3 H); MS (APPI) m/z 399 (M+1).

### Example 7

### 4-[7-(4-Methoxyphenyl)-3H-imidazo[4,5-b]pyridin-2-yl]-N-(2-morpholin-4-ylethyl)benzamide hydrochloride

### Example 7(a)

### 4-(7-Chloro-3H-imidazo[4,5-b]pyridin-2-yl)-N-(2-morpholin-4-ylethyl)benzamide

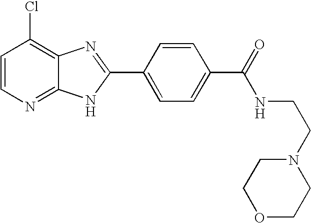

1,1′-Carbonylbis(1H-imidazole) (65 mg, 0.403 mmol) was added to 4-(7-chloro-3H-imidazo[4,5-b]pyridin-2-yl)benzoic acid (Example 5 (c)) (100 mg, 0.366 mmol) in dimethyl acetamide (2 mL) and the mixture was heated in a microwave reactor at +100° C. for 5 minutes. 2-Morpholin-4-ylethanamine (52 mg, 0.403 mmol) was added to the mixture and the resulting mixture was heated in microwave reactor at +120° C. for 5 minutes. The product mixture was used directly in the next step.

MS (APPI) m/z 386 (M+1).

### Example 7(b)

### 4-[7-(4-Methoxyphenyl)-3H-imidazo[4,5-b]pyridin-2-yl]-N-(2-morpholin-4-ylethyl)benzamide hydrochloride

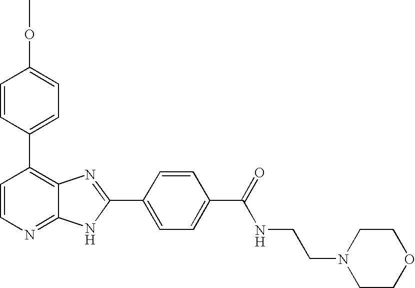

The title compound was prepared in accordance with the general method C, mixing the mixture of 4-(7-chloro-3H-imidazo[4,5-b]pyridin-2-yl)-N-(2-morpholin-4-ylethyl)benzamide (0.366 mmol) obtained from Example 7(a) with (4-methoxyphenyl)boronic acid (0.111 g, 0.733 mmol), PdCl₂(dppf)*DCM (0.015 g, 0.018 mmol) and sodium carbonate (0.116 g, 1.1 mmol), affording 0.011 g (5%) of the title compound.

¹H NMR (DMSO-d₆) δ ppm 11.29 (s, 1 H), 9.21 (s, 1 H), 8.49 (d, J=5.3 Hz, 1 H), 8.44 (d, J=8.3 Hz, 2 H), 8.25 (d, J=8.1 Hz, 2 H), 8.19 (d, J=8.6 Hz, 2 H), 7.65 (d, J=5.6 Hz, 1 H), 7.20 (d, J=8.8 Hz, 2 H), 4.01-3.84 (m, 7 H), 3.80-3.70 (m, 2 H), 3.56 (d, J=1.16 Hz, 2 H), 3.34 (m, 2 H), 3.22-3.03 (m, 2 H); MS (APPI) m/z 458 (M+1).

### Example 8

### 2-{4-[(4-Methylpiperazin-1-yl)carbonyl]phenyl}-7-[4-(trifluoromethoxy)phenyl]-3H-imidazo[4,5-b]pyridine hydrochloride

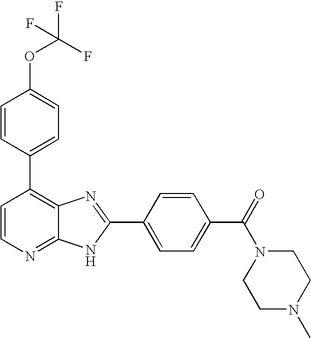

The title compound was prepared in accordance with the general method C using 7-chloro-2-{4-[(4-methylpiperazin-1-yl)carbonyl]phenyl}-3H-imidazo[4,5-b]pyridine (obtained from Example 5 (d)) (0.200 g, 0.563 mmol) and [4-(trifluoromethoxy)phenyl]boronic acid (0.232 g, 1.13 mmol), affording 0.046 g (15%) of the title compound.

1H NMR (DMSO-d₆) δ ppm 8.53-8.44 (m, 3 H), 8.40 (d, J=8.6 Hz, 2 H), 7.68 (d, J=8.3 Hz, 2 H), 7.64-7.55 (m, 3 H), 3.63-3.29 (m, 5 H), 3.19-3.02 (m, 3 H), 2.86-2.71 (m, 3 H);

MS (APPI) m/z 482 (M+1).

### Example 9

### 2-{4-[(4-Methylpiperazin-1-yl)carbonyl]phenyl}-7-pyridin-3-yl-3H-imidazo[4,5-b]pyridine hydrochloride

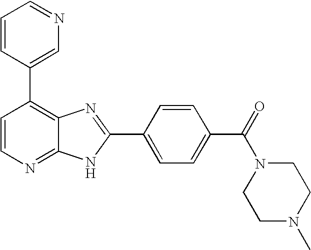

The title compound was prepared in accordance with the general method C using 7-chloro-2-{4-[(4-methylpiperazin-1-yl)carbonyl]phenyl}-3H-imidazo[4,5-b]pyridine (obtained from Example 5 (d)) (0.200 g, 0.563 mmol) and pyridin-3-ylboronic acid (0.139 g, 1.13 mmol), affording 0.069 g (26%) of the title compound.

¹H NMR (DMSO-d₆) δ ppm 9.91 (d, J=1.5 Hz, 1 H), 9.43 (d, J=8.1 Hz, 1 H), 9.00 (dd, J=5.6, 1.3 Hz, 1 H), 8.56 (d, J=5.1 Hz, 1 H), 8.45 (d, J=8.3 Hz, 2 H), 8.21 (dd, J=8.2, 5.6 Hz, 1 H), 7.88 (d, J=5.1 Hz, 1 H), 7.71 (d, J=8.3 Hz, 2 H), 3.53-3.26 (m, 5 H), 3.15 (s, 3 H), 2.79-2.72 (m, 3 H); MS (APPI) m/z 399 (M+1).

### Example 10

### 7-(2,4-Dimethoxyphenyl)-2-[4-(morpholin-4-ylcarbonyl)phenyl]-3H-imidazo[4,5-b]pyridine hydrochloride

### Example 10(a)

### 7-Chloro-2-[4-(morpholin-4-ylcarbonyl)phenyl]-3H-imidazo[4,5-b]pyridine

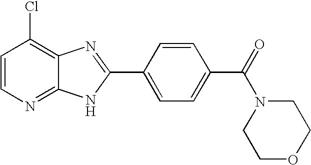

The title compound was prepared in accordance with the general method B using 4-(7-chloro-3H-imidazo[4,5-b]pyridin-2-yl)benzoic acid (obtained from Example 5(c)) (1.0 g, 3.66 mmol) and morpholine (0.38 g, 4.39 mmol), affording a crude yield of 1.67 g. The product was used without further purification in the next step.

¹H NMR (DMSO-d₆) δ ppm 8.33 (d, J=8.1 Hz, 2 H), 8.30 (d, J=5.1 Hz, 1 H), 7.62 (d, J=8.3 Hz, 2 H), 7.42 (d, J=5.3 Hz, 1 H), 3.80-3.20 (m, 9 H); MS (APPI) m/z 343 (M+1).

### Example 10(b)

### 7-(2,4-Dimethoxyphenyl)-2-[4-(morpholin-4-ylcarbonyl)phenyl]-3H-imidazo[4,5-b]pyridine hydrochloride

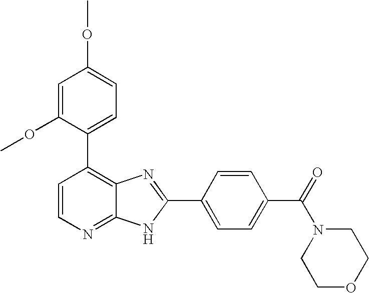

The title compound was prepared in accordance with the general method C using 7-chloro-2-[4-(morpholin-4-ylcarbonyl)phenyl]-3H-imidazo[4,5-b]pyridine (0.182 g, 0.532 mmol), which was obtained from Example 10(a), (2,4-dimethoxyphenyl)boronic acid (0.194 g, 1.06 mmol), PdCl₂(dppf)*DCM (0.022 g, 0.027 mmol) and sodium carbonate (0.169 g, 1.6 mmol), affording 0.023 g (9%) of the title compound.

¹H NMR (DMSO-d₆) δ ppm 8.57 (m, 1 H), 8.41-8.33 (m, 2 H), 7.81-7.71 (dd, J=33.7, 8.5 Hz, 1 H), 7.66 (d, J=8.3 Hz, 2 H), 7.63-7.53 (m 1 H), 6.82-6.70 (m, 2 H), 3.90 (s, 3 H), 3.87-3.86 (m, 2 H), 3.84 (s, 3 H), 3.77-3.24 (m, 8 H); MS (APPI) m/z 445 (N+1).

### Example 11

### 4-(2-{4-[(4-Methylpiperazin-1-yl)carbonyl]phenyl}-3H-imidazo[4,5-b]pyridin-7-yl)benzonitrile hydrochloride

### Example 11(a)

### Methyl 4-[7-(4-cyanophenyl)-3H-imidazo[4,5-b]pyridin-2-yl)benzonitrile hydrochloride

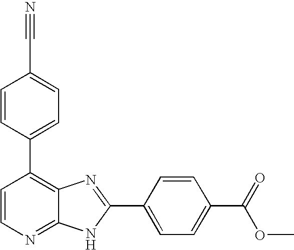

The title compound was prepared in accordance with the general method C with the exception that the base was obtained. Using methyl 4-(7-chloro-3H-imidazo[4,5-b]pyridin-2-yl)benzoate (obtained from Example 5 (b)) (0.330 g, 1.15 mmol) and (4-cyanophenyl)boronic acid (0.338 g, 2.30 mmol), the title compound was afforded in 0.395 g (97%) yield. The crude product was used in the next step without further purification.

¹H NMR (DMSO-d₆) δ ppm 8.66 (d, J=8.3 Hz, 2 H), 8.44 (d, J=8.6 Hz, 2 H), 8.33 (d, J=5.1 Hz, 1 H), 8.09 (d, J=8.6 Hz, 2 H), 8.02 (d, J=8.6 Hz, 2 H), 7.48 (d, J=5.1 Hz, 1 H), 4.00-3.78 (s, 3 H); MS (APPI) m/z 355 (M+1).

### Example 11((b)

### 4-(2-{4-[(4-Methylpiperazin-1-yl)carbonyl]phenyl}-3H-imidazo[4,5-b]pyridin-7-yl)benzonitrile hydrochloride

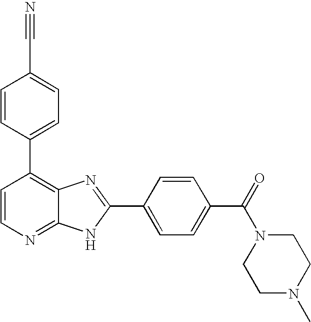

The title compound was prepared in accordance with the general method of Example 3(b) using methyl 4-[7-(4-cyanophenyl)-3H-imidazo[4,5-b]pyridin-2-yl]benzoate (0.100 g, 0.282 mmol), which was obtained from Example 11(a), N-methylpiperazine (2 mL) and HBTU (0.872 g, 2.3 mmol), affording the title compound in 0.072 g (51%) yield.

¹H NMR (DMSO-d₆) δ ppm 8.57 (d, J=8.3 Hz, 2 H), 8.48 (d, J=5.3 Hz, 1 H), 8.40 (d, J=8.3 Hz, 2 H), 8.12-8.04 (m, 2 H), 7.75-7.65 (m, 3 H), 3.70-3.22 (m, 5 H), 3.21-2.98 (m, 3 H), 2.78 (d, J=4.3 Hz, 3 H); MS (APPI) m/z 355 (M+1).

### Example 12

### 7-(4-Methoxyphenyl)-2-[4-(morpholin-4-ylmethyl)phenyl]-3H-imidazo[4,5-b]pyridine hydrochloride

### Example 12(a)

### 7-(4-Methoxyphenyl)-2-[4-(morpholin-4-ylcarbonyl)phenyl]-3H-imidazo[4, 5-b]pyridine

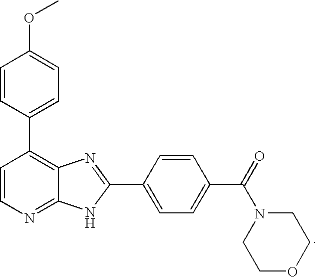

The title compound was prepared in accordance with the general method C with the exception that the base was obtained. Using 7-chloro-2-[4-(morpholin-4-ylcarbonyl)phenyl]-3H-imidazo[4,5-b]pyridine (obtained from Example 10(a)) (62 mg, 0.181 mmol), (4-methoxyphenyl)boronic acid (69 mg, 0.454 mmol), PdCl₂(dppt)*DCM (9.3 mg, 0.011 mmol) and sodium carbonate (72 mg, 0.68 mmol). The product was afforded in 33 mg (44%) yield.

MS (APPI) m/z 415 (M+1).

### Example 12(b)

### 7-(4-Methoxyphenyl)-2-[4-(morpholin-4-ylmethyl)phenyl]-3H-imidazo[4,5-b]pyridine hydrochloride

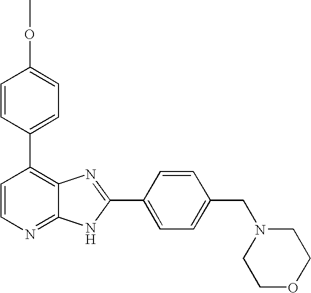

Borane-THF complex (1M, 1 mL) was added to 7-(4-methoxyphenyl)-2-[4-(morpholin-4-ylcarbonyl)phenyl]-3H-imidazo[4,5-b]pyridine (30 mg, 0.072 mmol) obtained from Example 12(a) at r.t.. After stirring at r.t. for 45 minutes, MeOH (1 mL) was added dropwise to the reaction mixture and the mixture was stirred for 2 h at r.t.. The solvent was evaporated in vacuo, and the base of the title compound was purified by preparative HPLC. The hydrochloride salt was prepared by dissolving the base in CH₂Cl₂/MeOH (2 mL, 9:1), 1M HCl in the (2 mL) was added and the precipitated was collected by filtration and dried, affording 17 mg (50%) of the title compound.

¹H NMR (D₂O) δ ppm 8.29 (d, J=6.1 Hz, 1 H), 8.02 (d, J=7.6 Hz, 2 H), 7.70 (d, J=8.6 Hz, 2 H), 7.61 (d, J=8.1 Hz, 2 H), 7.45 (d, J=6.1 Hz, 1 H), 6.97 (d, J=8.3 Hz, 2 H), 4.42 (s, 2 H), 4.21-3.96 (m, 2 H), 3.82 (s, 3 H), 3.89-3.68 (m, 2 H), 3.52-3.21 (m, 4 H); MS (APPI) m/z 401 (M+1).

### Example 13

### 2-{4-[4-Methylpiperazin-1-yl)carbonyl]phenyl}-7-[3-(morpholin-4-ylmethyl)phenyl]-1H-imidazo[4,5-b]pyridine

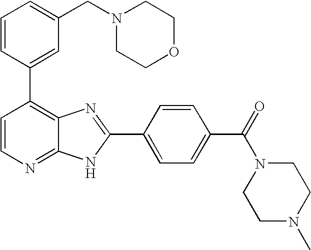

The title compound was prepared according to general method B using 4-{7-[3-(morpholin-4-ylmethyl)phenyl]-1H-imidazo[4,5-b]pyridine-2-yl}benzoic acid (crude, obtained from Example 13(f)), N-methyl piperazine (53 mg, 0.525 mmol), HBTU (239 mg, 0.63 mmol), DIPEA (202 mg, 1.57 mmol). The product was purified by semi-preparative chromatography and freeze-dried to provide the title compound as a white solid (10 mg, 4% over 4 steps).

¹H NMR (DMSO d-6) δ ppm 8.38 (d, 1 H), 8.35 (d, 2 H), 8.26 (s, 1 H), 8.22 (d, 1 H), 7.57-7.55 (m, 2 H), 7.54-7.50 (m, 2 H), 7.44 (d, 1 H), 5.75 (s, 2 H), 3.70-3.55 (m, 7 H), 2.45-2.40 (m, 4 H), 2.38-2.25 (m, 8 H); MS (ESI) m/z 495 (M−1); RT (HPLC, 254 nm) 3.33 min.

### Example 13(a)

### 4-Chloro-3-nitropyridin-2-amine

(Aust. J. Chem. 1982, 35, 2025.)

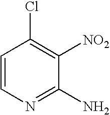

4-Chloro-2-aminopyridine (10 g, 77.5 mmol) was dissolved in concentrated sulfuric acid (100 mL) and using a salt-ice bath, was cooled to ca. −8° C. Fuming nitric acid was slowly added whilst stirring and at such a rate that a temperature ≦0° C. was maintained. The reaction mixture was then stirred for 20 minutes at ambient temperature and carefully is poured onto ice. Ammonium hydroxide (32%) was carefully added. Ice was used to maintain temperature <25° C. until the solution reached pH 3. The solid product was filtered, washed with water and re-crystallised from 1:1 water:EtOH. The solid was added in small portions to ice-cold concentrated sulfuric acid (200 mL) at a rate allowing a temperature of <4° C. to be maintained. Once addition was complete, the reaction mixture was allowed to reach ambient temperature. After 2.5 h at room temperature, 2 regioisomers (1:1), the 3 and 5-nitro compounds were observed (LCMS). The reaction mixture was poured onto ice and basified with ammonium hydroxide (32%). Filtration and subsequent washing with water provided the mixture of the 2 regioisomers. The products were dissolved in ethyl acetate to which was added heptane to effect trituration of the undesired regioisomer. Filtration of this isomer and evaporation of the ethyl acetate provided the desired product along with 15-20% of the undesired isomer. These isomers could also be separated using silica flash chromatography (Combiflashe system) with a suitable EtOAC/heptane gradient. (5 g, 37%).

MS (ESI) m/z 172 (M−1), 174. RT (LCMS, 254 nm) 2.2 min

### Example 13(b)

### 4-[3-(Morpholin-4-ylcarbonyl)phenyl]-3-nitropyridin-2-amine

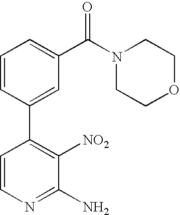

The title compound was prepared using general method C except purification of the title compound was achieved using silica flash chromatography (40-80% EtOAc:heptane) from 4-chloro-3-nitropyridin-2-amine (200 mg, 1.17 mmol), PdCl₂(dppf)*DCM (40 mg) potassium carbonate (800 mg, 5.75 mmol) and 3-(morpholin-4-ylcarbonyl)boronic acid (540 mg, 2.3 mmol) dissolved in THF:water (9:1) (6 mL). (140 mg, 37%); MS (ESI) m/z 329 (M+1) 327 (M−1), RT (LCMS, 254 nm) 2.25 min.

### Example 13(c)

### 4-[3-(Morpholin-4-ylcarbonyl)phenyl]pyridine-2,3-diamine

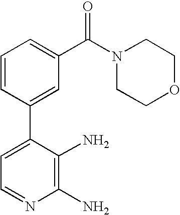

4-[3-(Morpholin-4-ylcarbonyl)phenyl]-3-nitropyridin-2-amine (140 mg, 0.42 mmol) obtained from Example 13(b) was dissolved in EtOAc:EtOH (8:2) (20 mL) and treated with 10% Pd/C (ca. 50 mg) under a hydrogen atmosphere at ambient temperature. Once is the reaction was complete (4-12 h) the reaction mixture was filtered through Celite® and evaporated to provide the title compounds as a solid (100 mg, 78%).

¹H NMR (DMSO d-6) δ ppm 7.55 (d, 1 H), 7.43-7.27 (m, 3 H), 6.81 (s, 1 H), 6.51 (d, 1 H), 4.6 (br s, 2 H), 3.81-3.25 (m, 10 H); MS (ESI) m/z 299 (M+1), RT (LCMS, 254 nm) 2.10 min.

### Example 13(d)

### 4-[3-(Morpholin-4-ylmethyl)phenyl]pyridine-2,3-diamine

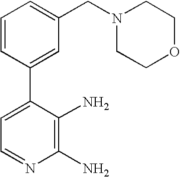

To a solution of 4-[3-(morpholin-4-ylcarbonyl)phenyl]pyridine-2,3-diamine (100 mg, 0.33 mmol, which was obtained from Example 13(c)) in anhydrous THF (10 mL) under N₂ was added LiAlH₄ (1M in THF, 1.32 mL, 1.2 mmol). The reaction mixture was heated at reflux for 3 h and then cooled to 0° C. Wet EtOAc (2 mL) was carefully added to quench the reaction. The reaction mixture was filtered and then evaporated. The resulting residue was stirred in dilute HCl (10 mL) and again filtered. This solution was basified (1M aq. K₂CO₃ solution) and the product was extracted into EtOAc (4-5×20 mL). The organic layer was dried and evaporated to provide the title compound (92 mg, 98%). This was deemed pure enough to take directly to the next step.

MS (ESI) m/z 285 (M+1), RT (LCMS, 254 nm) 2.00 min.

### Example 13(e)

### Methyl 4-{7-[3-(morpholin-4-ylmethyl)phenyl]-1H-imidazo[4,5-b]pyridine-2-yl}benzoate

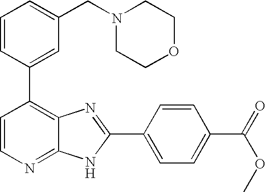

The title compound was prepared according to general method A from 4-[3-(morpholin-4-ylmethyl)phenyl]pyridine-2,3-diamine, which was obtained from Example 13(d) (149 mg, 0.53 mmol) monomethyl terephthalate (104 mg, 0.53 mmol), HBTU (219 mg, 0.57 mmol), DIPEA (75 mg, 0.74 mmol), acetonitrile (20 mL) and HOAc (5 mL). The title compound was taken directly to next step.

MS (ESI) m/z 445 (M+1) (intermediate hydroxyimine), 428 (M+1), RT (LCMS, 254 nm) 2.75 min.

### Example 13(f)

### 4-{7-[3-(Morpholin-4-ylmethyl)phenyl]-1H-imidazo[4,5-b]pyridine-2-yl}benzoic acid

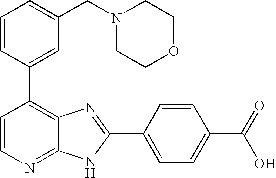

The title compound was prepared according to Example 5(c) from crude methyl 4-{7-[3-(morpholin-4-ylmethyl)phenyl]-1H-imidazo[4,5-b]pyridine-2-yl}benzoate from the previous step (Example 13(e)) treated with LiOH monohydrate (218 mg, 5.25 mmol) in dioxane/water (5 mL). The product was isolated as a crude mixture and taken directly to the final step.

MS (ESI) m/z 413 (M−1), 415 (+1), RT (LCMS, 254 nm) 2.02 min.

### Example 14

### N-(2-Cyanoethyl)-3-{2-[(4-methylpiperazin-1-yl)carbonyl]-3H-imidazo[4,5-b]pyridine-7-yl}benzamide

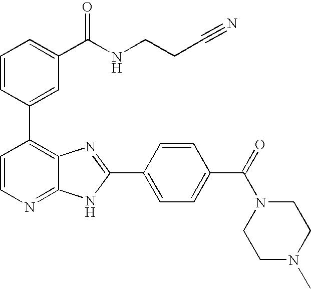

N-(2-Cyanoethyl)-3-(2-[(4-methylpiperazin-1-yl)carbonyl]-3-{[2-(trimethylsilyl)ethoxy]methyl}-3H-imidazo[4,5-b]pyridin-7-yl)benzamine (obtained from Example 14(e)) was dissolved in EtOH (5 mL) and treated with 5M HCl(aq) (1 mL). The reaction mixture was heated at reflux. After 45-60 min at reflux the reaction mixture was allowed to cool and was neutralised via addition of saturated aqueous potassium carbonate solution. The reaction mixture was extracted with EtOAc (2×10 mL) evaporated and purified by semi-preparative chromatography. Freeze-drying provided the title compound (2 mg, 6% over 4 steps).

¹H NMR (DMSO d-6) δ ppm 8.67 (s, 1 H), 8.45 (s, 1 H), 8.22 (s, 1 H), 8.12-8.08 (m, 2 H), 7.89-7.85 (m, 1 H), 7.54 (t, 1 H), 7.51-7.45 (m, 3 H), 3.75 (s, 2 H), 3.73 (dd, 2 H), 3.45 (s, 2 H), 2.71 (t, 2 H), 2.50 (s, 2 H), 2.28 (s, 2 H), 2.08 (s, 3 H); MS (ESI) m/z 494 (M+1), 492

### Example 14(a)

### Methyl 4-(7-iodo-3H-imidazo[4,5-b]pyridin-2-yl)benzoate

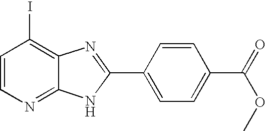

Methyl 4-(7-chloro-3H-imidazo[4,5-b]pyridin-2-yl)benzoate (6.0 g, 21 mmol) was suspended in anhydrous MeOH (25 mL) and treated with HCl (1.0 M in diethyl ether) until all of the starting material had dissolved. Diethyl ether was then added until a precipitate was formed which was filtered and vacuum dried (5.5 g). NaI (11.5 g, 76.4 mmol) was added and the dry mixture was taken up in MeCN (40 mL) and placed in a suitable microwave vial. MW irradiation (+160° C., 10 min) provided the title compound (4 g, 51%) which was filtered.

MS (ESI) m/z 380 (M+1); RT (HPLC) 4.02 min.

### Example 14(b)

### Methyl 4-(7-iodo-3-{[2-(trimethylsilyl)ethoxy]methyl}-3H-imidazo[4,5-b]pyridine-2-yl)benzoate

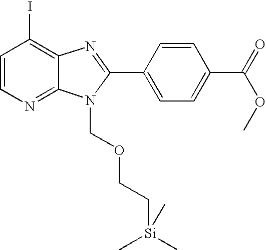

To a suspension of NaH (37 mg, 0.91 mmol) in anhydrous DMF (10 mL) was added a solution of methyl 4-(7-iodo-3H-imidazo[4,5-b]pyridin-2-yl)benzoate (312 mg, 0.83 mmol, which was obtained from Example 14(a)) in DMF (2 mL). The reaction mixture was allowed to stir for 90 min. at which point SEM-Cl (138 mg, 0.83 mmol) was added. After 1.5 h the reaction was complete. Water was added (5 mL) and the product was extracted into EtOAc (20 mL). The organic phase was washed with water (4×50 mL) to remove the DMF, dried and evaporated. Silica flash chromatography (Combiflash® system) (20%-80% EtOAc/heptane gradient) provided the title compound (30 mg, 7%). MS (ESI) m/z 510 (M+1); RT (HPLC) 5.35 min.

### Example 14(c)

### Methyl 4-(7-(3-{[(2-cyanoethyl)amino]carbonyl}phenyl)-3-{[2-(trimethylsilyl)ethoxy]methyl}-3H-imidazo[4,5-b]pyridin-2-yl)benzoate

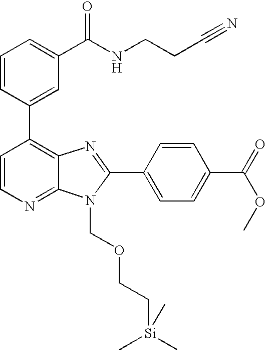

The title compound was prepared according to general method C from methyl 4-(7-iodo-3-{[2-(trimethylsilyl)ethoxy]methyl}-3H-imidazo[4,5-b]pyridine-2-yl)benzoate obtained from Example 14(b) (30 mg, 0.06 mmol), PdCl₂(dppt)*DCM (5 mg) potassium carbonate (33 mg, 0.24 mmol) and 3-{[(2-cyanoethyl)amino]carbonyl}phenyl boronic acid (26 mg, 0.12 mmol) dissolved in THF:water (9:1) (4 mL). The reaction mixture was washed with water and extraced with EtOAc (2×10 mL) dried and evaporated. The crude product was taken directly to the next step.

MS (ESI) m/z 554 (M−1), 556 (M+1); RT (HPLC) 5.06 min.

### Example 14(d)

### 4-(7-(3-{[(2-Cyanoethyl)amino]carbonyl}phenyl)-3-{[2-(trimethylsilyl)ethoxy]methyl}-3H-imidazo[4,5-b]pyridin-2-yl)benzoic acid

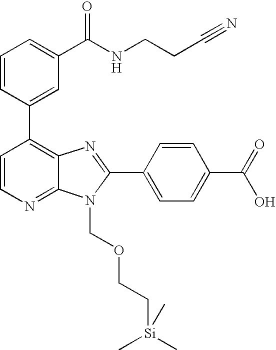

Methyl 4-(7-iodo-3-{[2-(trimethylsilyl)ethoxy]methyl}-3H-imidazo[4,5-b]pyridine-2-yl)benzoate obtained from Example 14(c) was dissolved in THF:water (9:1) (4 mL), treated with LiOH monohydrate (0.6 mmol) and subjected to microwave irradiation (+120° C., 10 min). The reaction mixture was neutralised with 2 M HCl and extracted with EtOAc (2×20 mL). The organic phase was separated, dried and evaporated and the product taken directly to the next step.

MS (ESI) m/z 542 (M+1); RT (LCMS) 2.75 min.

### Example 14(e)

### N-(2-Cyanoethyl)-3-(2-[(4-methylpiperazin-1-yl)carbonyl]-3-{[2-(trimethylsilyl)ethoxy]methyl}-3H-imidazo[4,5-b]pyridin-7-yl)benzamine

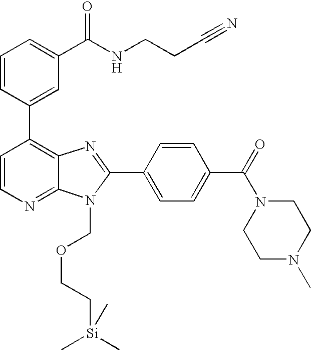

The title compound was prepared according to general method B from 4-(7-(3-{[(2-cyanoethyl)amino]carbonyl}phenyl)-3-{[2-(trimethylsilyl)ethoxy]methyl}-3H-imidazo[4,5-b]pyridin-2-yl)benzoic acid (crude from previous step (Example 14(d)), N-methyl piperazine (6 mg, 0.06 mmol), HBTU (23 mg, 0.06 mmol), DIPEA (8 mg, 0.06 mmol) in MeCN (5 mL). The solvent was evaporated and the residue taken up in EtOAc (20 mL), washed with water (10 mL) dried and evaporated. The crude product was taken directly to the final step.

MS (ESI) m/z 622 (M−1).

### Example 15

### 7-{3-[2-(2-Methoxyethoxy)ethoxy]phenyl}-2-{4-[(4-methylpiperazin-1-yl)carbonyl]phenyl}-3H-imidazo[4,5-b]pyridine

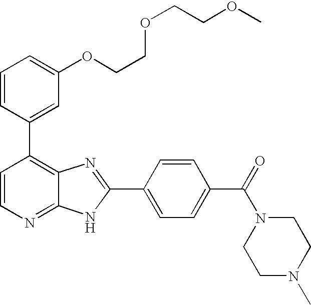

The title compound was furnished according to the procedure described in Example 14 but using 7-{3-[2-(2-methoxyethoxy)ethoxy]phenyl}-2-{4-[(4-methylpiperazin-1-yl)carbonyl]phenyl}-3-{[2-(trimethylsilyl)ethoxy]methyl}-3H-imidazo[4,5-b]pyridine (obtained from Example 15(f)) via treatment with 5M HCl (aq) and purification by semi-preparative chromatography (4 mg, 8% over 2 steps).

¹H NMR (DMSO d-6) δ ppm 8.41 (d, 1 H), 8.28 (d, 2 H), 7.91 (s, 1 H), 7.82 (d, 1 H), 7.60 (d, 2 H), 7.50-7.37 (m, 2 H), 7.00 (m, 1 H), 4.22 (m, 2 H), 3.87 (m, 2 H), 3.75 (s, 2 H), 3.68 (m, 2 H), 3.52 (m, 2 H), 3.49 (s, 2 H), 3.34 (s, 3 H), 2.50 (s, 2 H), 2.32 (s, 2 H), 2.28 (s, 3 H); MS (ESI) m/z 516 (M+1), 514 (M−1); RT (HPLC, 254 nm) 3.29 min.

### Example 15(a)

### Methyl 4-(7-[3-(benzyloxy)phenyl]-3H-imidazo[4,5-b]pyridine-2-yl)benzoate

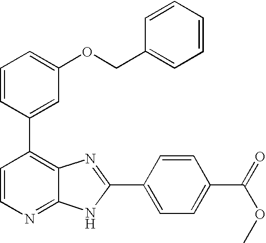

The title compound was prepared according to general method C from methyl 4-(7-iodo-3H-imidazo[4,5-b]pyridin-2-yl)benzoate (435 mg, 1.15 mmol, obtained from Example 14(a)), PdCl₂(dppf)*DCM (47 mg, 0.057 mmol), potassium carbonate (635 mg, 4.60 mmol) and 3-(benzyloxy)phenyl boronic acid (525 mg, 2.30 mmol) dissolved in THF:water (9:1) (15 mL). The reaction mixture was washed with water and extraced with EtOAc (2×25 mL) dried and evaporated. The crude product was isolated via silica flash chromatography (Combiflash® system, 20-60% EtOAc:heptane gradient), (100 mg, 20%).

¹H NMR (DMSO d-6) δ ppm 8.52-8.48 (m, 3 H), 8.28 (d, 2 H), 8.21-8.19 (m, 1 H), 8.10 (d, 1 H), 7.71 (m, 1 H), 7.64 (m, 3 H), 7.52 (m, 2 H), 7.48 (m, 1 H), 7.27 (d, 2 H), 5.38 (s, 2 H), 4.00 (s, 3 H); MS (ESI) m/z 434 (M−1); RT (LCMS) 3.52 min.

### Example 15(b)

### Methyl 4-(7-[3-(benzyloxy)phenyl]-3-{[2-(trimethylsilyl)ethoxy]methyl}-3H-imidazo[4,5-b]pyridine-2-yl)benzoate

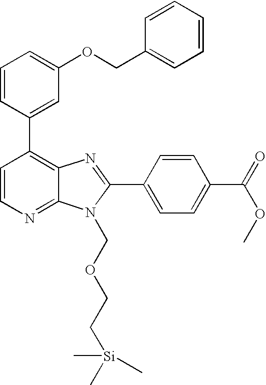

The title compound was prepared according the procedure described in Example 14(b) from methyl 4-(7-[3-(benzyloxy)phenyl]-3H-imidazo[4,5-b]pyridine-2-yl)benzoate (100 mg, 0.23 mmol, obtained from Example 15(a)), NaH (9.1 mg, 0.23 mmol) and SEM-Cl (40 mg, 0.24 mmol), (50 mg, 39%).

¹H NMR (DMSO d-6) δ ppm 8.76 (d, 2 H), 8.21 (d, 2 H), 7.98 (m, 2 H), 7.51 (m, 3 H), 7.42 (m, 3 H), 7.31 (m, 1 H), 7.25 (s, 1 H), 7.18 (d, 1 H), 6.20 (s, 2H), 5.24 (s, 2 h), 3.98 (s, 3 H), 3.78 (m, 2 H), 1.15 (m, 2 H), 0.20 (s, 9 H); MS (ESI) m/z 566 (M+1).

### Example 15(c)

### 4-(7-[3-(Benzyloxy)phenyl]-3-{[2-(trimethylsilyl)ethoxy]methyl}-3H-imdazo[4,5-b]pyridine-2-yl)benzoic acid

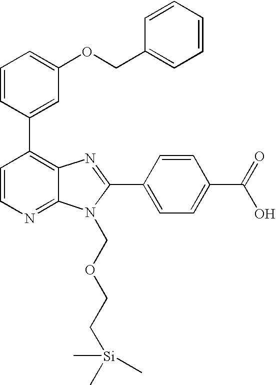

Methyl 4-(7-[3-(benzyloxy)phenyl]-3-{[2-(trimethylsilyl)ethoxy]methyl}-3H-imidazo[4,5-b]pyridine-2-yl)benzoate (50 mg, 0.09 mmol, obtained from Example 15(b)) and LiOH monohydrate (36 mg, 0.86 mmol) were suspended in THF:water (9:1), (5 mL) and subjected to heating by MW irradiation (+120° C., 10 min). The reaction mixture was adjusted to pH 7 via addition of 2M HCl (aq) and extracted with EtOAc (2×10 mL). The organic phase was dried and evaporated to provide the title compound(19 mg, 40%).

MS (ESI) m/z 550 (M−1); RT (HPLC, 254 nm) 4.53 min.

### Example 15(d)

### 7-[3-(Benzyloxy)phenyl]-2-{4-[(methylpiperazin-1-yl)carbonyl]phenyl}-3-{[2-(trimetlylsilyl)ethoxy]methyl{3H-imidazo[4,5-b]pyridine

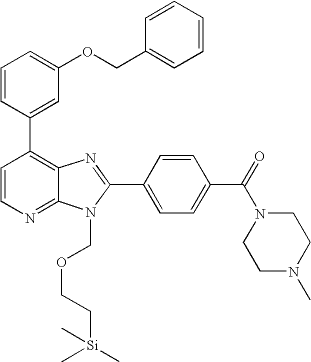

The title compound was prepared according to general method B from 4-(7-[3-(benzyloxy)phenyl]-3-{[2-(trimethylsilyl)ethoxy]methyl}-3H-imdazo[4,5-b]pyridine-2-yl)benzoic acid (19 mg, 0.036 mmol, obtained from Example 15(c)), N-methyl piperazine (4.2 mg, 0.043 mmol), HBTU (13 mg, 0.035 mmol), DIPEA (5.4 mg, 0.043 mmol) in MeCN (5 mL). The solvent was evaporated and the residue taken up in EtOAc (20 mL), washed with water (10 mL) dried and evaporated. The product was isolated via silica flash chromatography (Combiflash® system, 0-50% EtOAc:heptane gradient), (20 mg, 91%).

MS (ESI) m/z 634 (M+1); RT (HPLC, 254 nm) 5.65 min.

### Example 15(e)

### 3-(2-{4-[(4-Methylpiperazin-1-yl)carbonyl]phenyl}-3-{[2-(trimethylsilyl)ethoxy]methyl)-3H-imidazo[4,5-b]pyridin-7-yl)phenol

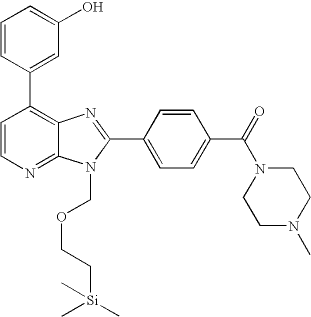

7-[3-(Benzyloxy)phenyl]-2-{4-[(methylpiperazin-1-yl)carbonyl]phenyl}-3-{[2-(trimethylsilyl)ethoxy]methyl}3H-imidazo[4,5-b]pyridine (obtained from Example 15(d) was dissolved in EtOAc:EtOH (8:2, 10 mL), treated with Pd/C (10%, 5 mg) and stirred at RT under a hydrogen atmosphere. After 4-6 h the reaction appeared complete. The reaction mixture was filtered though Celite® and evaporated to provide the title compound (10 mg, 59%).

MS (ESI) m/z 544 (M+1), 542 M−1); RT (HPLC, 254 nm) 4.26 min.

### Example 15(f)

### 7-{3-[2-(2-Methoxyethoxy)ethoxy]phenyl}-2-(4-[(4-methylpiperazin-1-yl)carbonyl]phenyl)-3-([2-(trimethylsilyl)ethoxy]methyl}-3H-imidazo[4,5-b]pyridine

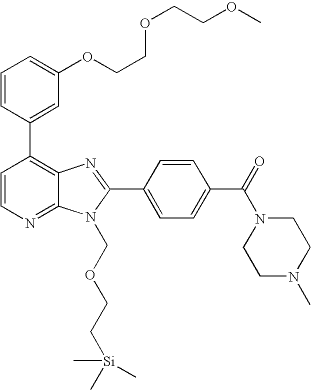

To a suspension of NaH (5 mg, 0.12 mmol) in anhydrous DMF (5 mL) under nitrogen at ambient temperature was added 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (45 mg, 0.10 mmol, obtained from Example 15(e)). The reaction mixture was stirred for 15 min and then treated with (36 mg, 0.19 mmol). After 1 h the reaction mixture was treated with water (1 mL) and extracted into EtOAc (2×10 mL). The organic phase was washed with water (4×20 mL) dried and evaporated. The title compound was taken crude to the final step. MS (ESI) m/z 646 (M+1); RT (HPLC, 254 nm) 4.90 min.

### Example 16

### 3-[3-(2-{4-[(4-Methylpiperazin-1-yl)carbonyl]phenyl}-3H-imidazo[4,5-b]pyridin-7yl)propan-1-ol

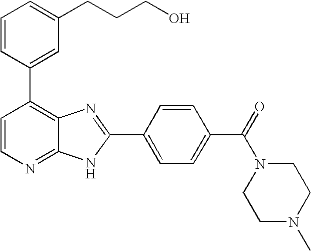

The title compound was furnished according to the procedure described in Example 14 but using 3-[3-(2-{4-methylpiperazin-1-yl)carbonyl]phenyl}-3-{[2-(trimethylsilyl)ethoxy]methyl}-3H-imidazo[4,5-b]pyridin-7-yl)phenyl]propan-1-ol (obtained from Example 16(c)) via treatment with 5M HCl (aq) and purification by semi-preparative chromatography (4 mg, 5% over 2 steps).

1H NMR (DMSO d-6) δ ppm 8.55 (d, 1 H), 8.46 (d, 2 H), 8.36-8.21 (m, 3 H), 7.72-7.61 (m, 3 H), 7.60-7.50 (m, 1 H), 7.47 (d, 1 H), 4.70 (t, 1 H), 3.79 (s, 2 H), 3.64-3.55 (m, 2 H), 3.55 (s, 2 H, obscured), 2.92-2.81 (m, 2 H), 2.51-2.36 (m, 4 H), 2.33 (s, 3 H), 1.97-1.86 (m,

### Example 16(a)

### Methyl 4-(7-[3-(3-hydroxypropyl)phenyl]-3-{[2-(trimethylsilyl)ethoxy]methyl)-3H-imidazo[4,5-b]pyridine-2-yl)benzoate

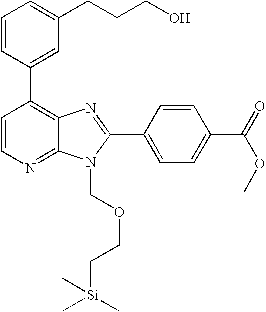

The title compound was furnished according to according to general method C from methyl 4-(7-iodo-3-{[2-(trimethylsilyl)ethoxy]methyl}-3H-imidazo[4,5-b]pyridine-2-yl)benzoate (100 mg, 0.26 mmol, obtained from Example 14(a)), PdCl₂(dppf)*DCM (10 mg, 0.013 mmol) potassium carbonate 146 mg, 1.05 mmol) and 3-(3-hydroxypropyl)phenyl boronic acid (95 mg, 0.53 mmol) dissolved in THF:water (9:1) (10 mL). The reaction mixture was washed with water and extracted with EtOAc (2×20 mL) dried and evaporated. The crude product was taken directly to the next step.

MS (ESI) m/z 518 (M+1); RT (HPLC, 254 nm) 5.34 min.

### Example 16(b)

### 4-(7-[3-(3-Hydroxypropyl)phenyl]-3-{[2-(trimethylsilyl)ethoxy]methyl)-3H-imidazo[4,5-b]pyridine-2-yl)benzoic acid

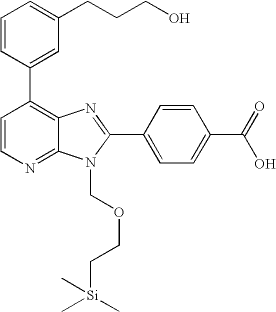

The title compound was furnished as described in Example 15(c) from crude methyl 4-(7-[3-(3-hydroxypropyl)phenyl]-3-{[2-(trimethylsilyl)ethoxy]methyl}-3H-imidazo[4,5-b]pyridine-2-yl)benzoate (obtained from Example 16(b)) and LiOH monohydrate (108 mg, 2.64 mmol), (80 mg, 60%).

MS (ESI) m/z 502 (M−1); RT (LCMS, 254 nm) 2.80 min.

### Example 16(c)

### 3-[3-(2-(4-Methylpiperazin-1-yl)carbonyl]phenyl -3-[2-(trimethylsilyl)ethoxy]methyl)-3H-imidazo[4,5-b]pyridin-7-yl)phenyl]propan-1-ol

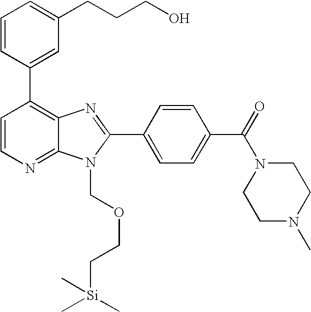

The title compound was furnished according to general method B from 4-(7-[3-(3-hydroxypropyl)phenyl]-3-{[2-(trimethylsilyl)ethoxy]methyl}-3H-imidazo[4,5-b]pyridine-2-yl)benzoic acid (80 mg, 0.16 mmol, obtained from Example 16(b)), N-methylpiperazine (16 mg, 0.16 mmol), DIPEA (25 mg, 0.19 mmol) and HBTU (60 mg, 0.16 mmol). The crude product was taken directly to the final step.

MS (ESI) m/z 586 (M+1); RT (HPLC, 254 nm) 4.38 min.

### Example 17

### 7-[3-(3-Methoxypropoxy)phenyl]-2-{4-[(4-methylpiperazin-1-yl)carbonyl]phenyl}-3H-imidazo[4,5-b]pyridine

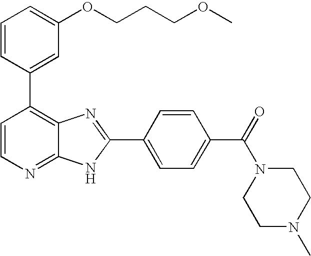

The title compound was furnished as described in Example 14 from [4-[2-[3-(3-methoxypropoxy)phenyl]-7-(2-trimethylsilylethoxymethyl)-5,7,9-triazabicyclo[4.3.0]nona-1,3,5,8-tetraen-8-yl]phenyl]-(4-methylpiperazin-1-yl)-methanone (obtained from Example 17(a)) via treatment with 5M HCl (aq) and purification by semi-preparative chromatography (4 mg, 3% over 2 steps).

¹H NMR (DMSO d-6) δ ppm 8.40-8.34 (m, 1 H), 8.31 (d, 2 H), 7.92 (s, 1 H), 7.84-7.76 (m, 1H), 7.59 (d, 2 H), 7.48-7.37 (m, 2 H), 6.96 (d, 1 H), 4.16 (t, 2 H), 3.73-3.85 9m, 2 H), 3.55 (t, 2 H), 3.50-3.40 (m, 2 H), 3.32 (s, 3 H), 2.53-2.42 (m, 2 H), 2.42-2.30 (m, 2 H), 2.28 (s, 3 H), 2.11-2.04 (m, 2 E); MS (ESI) m/z 486 (M+1), 484 (M−1); RT (HPLC, 254 nm) 2.15 min.

### Example 17(a)

### [4-[2-[3-(3-methoxypropoxy)phenyl]-7-(2-trimethylsilylethoxymethyl)-5, 7, 9-triazabicyclo[4.3.0]nona-1,3,5,8-tetraen-8-yl]phenyl]-(4-methylpiperazin-1-yl)-methanone

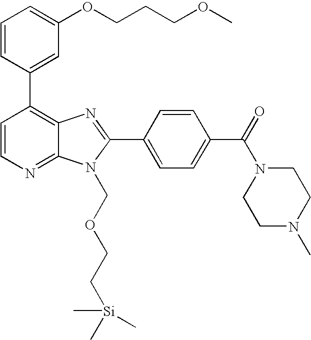

The title compound was furnished as described in Example 15(f) from 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (150 mg, 0.27 mmol, obtained from Example 15(e)), 1-bromo-3-methoxypropane (61 mg, 0.41 mmol), NaH (60% in mineral oil, 17 mg, 0.41 mmol) and DMF (5 mL). The crude product was taken directly to the final step.

MS (ESI) m/z 616 (M+1); RT (HPLC, 254 nm) 5.02 min.

### Example 18

### N-{3-[3-(2-{4-[4-Methylpiperazin-1-yl)carbonyl]phenyl}-3H-imidazo[4,5-b]pyridine-7-yl)phenoxy]propyl}acetamide

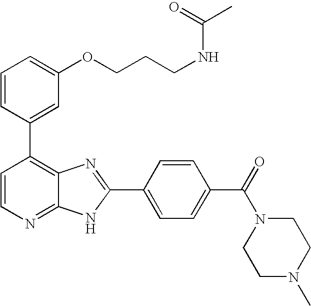

The title compound was furnished as previously described in Example 14 from N-[3-[3-[8-[4-(4-methylpiperazin-1-yl)carbonylphenyl]-7-(2-trimethylsilylethoxymethyl)-5,7,9-triazabicyclo[4.3.0]nona-1,3,5,8-tetraen-2-yl]phenoxy]propyl]acetamide (obtained from Example 18(b)) via treatment with 5M HCl (aq) and purification by semi-preparative chromatography (11 mg, 3% over 2 steps).

¹H NMR (CDCl₃) δ ppm 8.31 (s, 1 H), 8.19 (d, 2 H), 7.86 (br s, 1 H), 7.72 (br s, 1 H), 7.52 (d, 2 H), 7.39-7.35 (m, 2 H), 5.90 (s, 1 H), 4.13 (t, 2 H), 3.90-3.68 (m, 2 H), 3.55-3.30 (m, 4 H), 2.51-2.30 (m, 4 H), 2.29 (s, 3 H), 2.08 (s, 3 H), 1.52-1.48 (m, 2 H); MS (ESI) m/z 511 (M−1); RT (HPLC, 254 nm) 1.82 min.

### Example 18(a)

### N-(3-Bromopropyl)acetamide

(J. Het. Chem 1999, 36, 105)

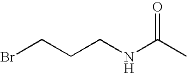

To a solution of 3-bromopropylamine hydrochloride (1.0 g, 4.58 mmol) in toluene (25 mL) at 0° C. was added, successively, triethylamine (0.46 g, 4.58 mmol) and acetic anhydride (0.46 g, 4.58 mmol). The reaction mixture was then allowed to stir overnight at room temperature. The solvent was removed and the residue dissolved in EtOAc (35 mL). The organic solution was washed successively with water (3×20 mL), brine (3×20 mL) then dried and evaporated to provide the title compound exclusively (0.8 g, 97%).

¹H NMR (DMSO d-6) δ ppm 6.50-6.29 (m, 1 H), 3.38 (t, 2 H), 3.28 (dd, 2 H), 2.07-1.98 (m, 2 H), 1.94 (s, 3 H)

### Example 18(b)

### N-[3-[3-[8-[4-(4-methylpiperazin-1-yl)carbonylphenyl]-7-(2-trimethylsilylethoxymethyl)-5,7,9-triazabicyclo[4.3.0]nona-1,3,5,8-tetraen-2-yl]phenoxy]propyl]acetamide

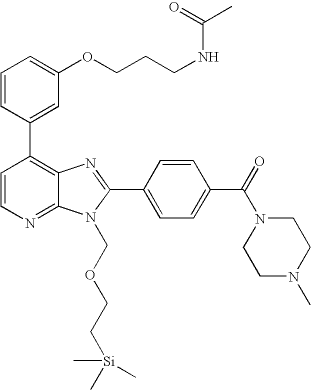

The title compound was furnished as previously described in Example 15(f) from 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (410 mg, 0.75 mmol, obtained from Example 15(e)), N-(3-bromopropyl)acetamide (135 mg, 0.75 mmol, obtained from Example 18(a)), NaH (60% in mineral oil, 30 mg, 0.75 mmol) and DMF (15 mL). The crude product was taken directly to the next step.

MS (ESI) m/z 643 (M+1); RT (HPLC, 254 nm) 4.29 min.

### Example 19

### 4-[3-(2-{4-[(4-Methylpiperazin-1-yl)carbonyl]phenyl}-3H-imidazo[4,5-b]pyridine-7-yl)phenoxy]butanenitrile

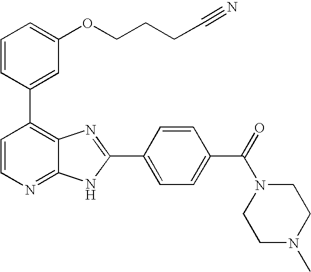

The title compound was furnished as previously described in Example 14 from 4-[3-(2-{4-[(4-methylpiperazin-1-yl)carbonyl]phenyl}-3-{[2-(trimethylsilyl)ethoxy]methyl}-3H-imidazo[4,5-b]pyridine-7-yl)phenoxy]butanenitrile ((obtained from Example 19(a)) via treatment with 5M HCl (aq) and purification by semi-preparative chromatography (5 mg, 6% over 2 steps).

¹H NMR (CDCl₃) δ ppm 8.41 (d, 1 H), 8.35 (d, 2 H), 8.01-7.94 (m, 1 H), 7.87-7.77 (m, 1 H), 7.60 (d, 2 H), 7.53-7.42 (m, 2 H), 6.99 (d, 1 H), 4.23 (t, 2 H), 3.92-3.75 (m, 2 H), 3.59-3.41 (m, 2 H), 2.65 (t, 2 H), 2.58-2.26 (m, 4 H), 2.31 (s, 3 H), 2.29-2.14 (m, 2 H); MS (ESI) m/z 481 (M+1), 479 (M−1); RT (HPLC, 254 nm) 2.00 min.

### Example 19(a)

### 4-[3-(2-{4-[(4-Methylpiperazin-1-yl)carbonyl]phenyl}-3-{[2-(trimethylsilyl)ethoxy]methyl)-3H-imidazo[4,5-b]pyridine-7-yl)phenoxy]butanenitrile

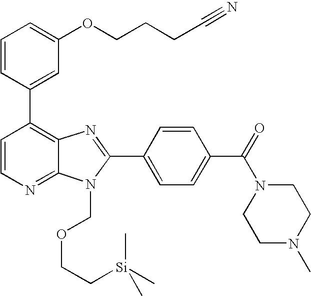

The title compound was furnished as previously described in Example 15(f) from 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (100 mg, 0.184 mmol, obtained from Example 15(e)), 4-bromobutanenitrile (41 mg, 0.28 mmol), NaH (60% in mineral oil, 11 mg, 0.28 mmol) and DMF (5 mL). The crude product was taken directly to the final step.

MS (ESI) m/z 611 (M+1); RT (HPLC, 254 nm) 1.82 min.

### Example 20

### 3-[3-(2-{4-[(4-Methylpiperazin-1-yl)carbonyl]phenyl}-3H-imidazo[4,5-b]pyridine-7-yl)phenoxy]propan-1-ol

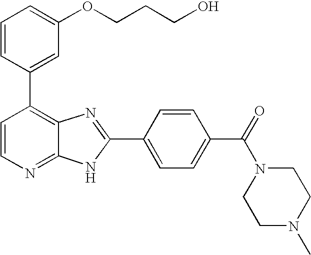

The title compound was furnished as previously described in Example 14 from 3-[3-(2-{4-[(4-methylpiperazin-1-yl)carbonyl]phenyl}-3-{[2-(trimethylsilyl)ethoxy]methyl}-3H-imidazo[4,5-b]pyridine-7-yl)phenoxy]propan-1-ol (obtained from Example 20(a)) via treatment with 5M HCl (aq) and purification by semi-preparative chromatography (2 mg, 3% over 2 steps).

¹H NMR (CDCl₃) δ ppm 8.35-8.28 (m, 1 H), 8.24 (d, 2 H), 7.93-7.83 (m, 1 H), 7.1-7.69 (m, 1 H), 7.54 (d, 2 H), 7.46-7.33 (m, 2 H), 7.00 (d, 1 H), 4.22 (t, 2 H), 3.85 (t, 2 H), 3.82-3.72 (m, 2 H), 3.51-3.32 (m, 2 H), 2.53-2.31 (m, 4 H), 2.25 (s, 3 H), 2.11-2.01 (m, 2 H);

MS (ESI) m/z 472 (M+1), 470 (M−1), RT (LCMS, 254 nm) 1.84 min.

### Example 20(a)

### 3-[3-(2-{4-[(4-Methylpiperazin-1-yl)carbonyl]phenyl)-3-{[2-(trimethylsilyl)ethoxy]methyl}-3H-imidazo[4,5-b]pyridine-7-yl)phenoxy]propan-1-ol

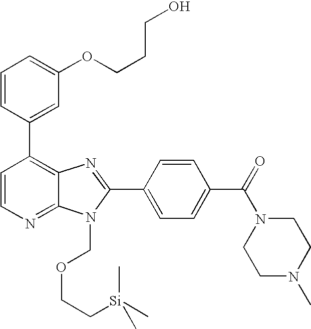

The title compound was furnished as previously described in Example 15(f) from 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (100 mg, 0.184 mmol, obtained from Example 15(e)), 3-bromopropanol (39 mg, 0.28 mmol), NaH (60% in mineral oil, 11 mg, 0.28 mmol) and DMF (5 mL). The crude product was taken directly to the final step.

MS (ESI) m/z 602 (M+1)

### Example 21

### 3-[3-(2-{4-[(4-Methylpiperazin-1-yl)carbonyl]phenyl}-3H-imidazo[4,5-b]pyridine-7-yl)phenoxy]acetonitrile

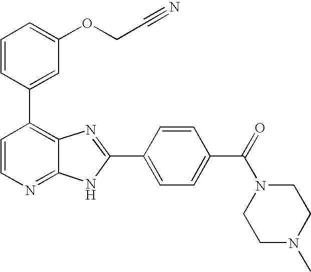

The title compound was furnished as previously described in Example 14 from 3-[3-(2-{4-[(4-methylpiperazin-1-yl)carbonyl]phenyl}-3-{[2-(trimethylsilyl)ethoxy]methyl}-3H-imidazo[4,5-b]pyridine-7-yl)phenoxy]acetonitrile (obtained from Example 21(a)) via treatment with 5M HCl (aq) and purification by semi-preparative chromatography (4 mg, 3% over 2 steps).

¹H NMR (CDCl₃) δ ppm 8.39-8.29 (m, 1 H), 8.25 (d, 2 H), 8.15-8.01 (m, 1 H), 7.91-7.80 (m, 1 H), 7.55 (d, 2 H), 7.51-7.39 (m, 2 H), 7.05 (d, 1 H), 4.80 (s, 2 H), 3.90-3.69 (m, 2 H), 3.58-3.35 (m, 2 H), 2.54-2.33 (m, 4 H), 2.28 (s, 3 H); MS (ESI) m/z 451 (M−1); RT (LCMS, 254 nm) 3.21 min.

### Example 21(a)

### 3-[3-(2-{4-[(4-Methylpiperazin-1-yl)carbonyl]phenyl}-3-{[2-(trimethylsilyl)ethoxy]methyl]-3H-imidazo[4,5-b]pyridine-7-yl)phenoxy]acetonitrile

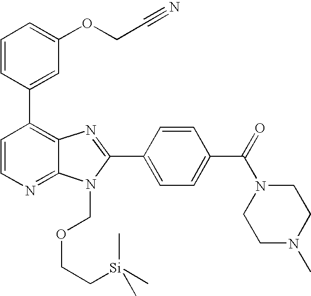

The title compound was furnished as previously described in Example 15(f) from 3-(2-{4-[(4-methylpiperazin-1-yl)carbonyl]phenyl}-3-{[2-(trimethylsilyl)ethoxy]methyl}-3H-imidazo[4,5-b]pyridin-7-yl)phenol (200 mg, 0.37 mmol, obtained from Example 15(e)), 1-bromoacetonitrile (67 mg, 0.55 mmol), NaH (60% in mineral oil, 22 mg, 0.55 mmol) and DMF (10 mL). The crude product was taken directly to the final step.

MS (ESI) m/z 583 (M+1)

### Example 22

### 7-(4-Methoxyphenyl)-2-{5-[(4-methylpiperzin-1-yl)carbonyl]pyridine-2-yl}-3H-imidazo[4,5-b]pyridine

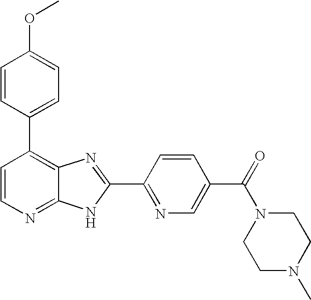

The title compound was prepared according to the procedure described in Example 3(b) from methyl 6-[7-(4-methoxyphenyl)-3H-imidazo[4,5-b]pyridine-2-yl]nicotinate (obtained from Example 22(a)) (100 mg, 0.27 mmol), N-methyl piperazine (27 mg, 0.27 mmol) and HBTU (102 mg, 0.27 mmol). Purification by semi-preparative HPLC provided 7-(4-methoxyphenyl)-2-{5-[(4-methylpiperzin-1-yl)carbonyl]pyridine-2-yl}-3H-imidazo[4,5-b]pyridine as a white solid (40 mg, 31%).

¹H NMR (DMSO d-6) δ ppm 8.91-8.75 (m, 1 H), 8.54 (d, 1 H), 8.44 (d, 2 H), 8.32 (d, 1 H), 8.25-8.10 (m, 1 H), 8.00 (s, 1 H), 7.62 (d, 1 H), 7.21 (d, 2 H), 3.90 (s, 3 H), 3.65-2.54 (m, 4 H), 3.11-3.04 (m, 4 H), 2.11 (s, 3 H); MS (ESI) m/z 429 (M+1); RT (LCMS) 3.49 min.

### Example 22(a)

### Methyl 6-[7-(4-methoxyphenyl)-3H-imidazo[4,5-b]pyridine-2-yl]nicotinate

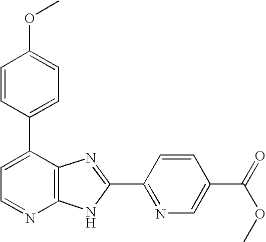

Methyl 6-[7-(4-methoxyphenyl)-3H-imidazo[4,5-b]pyridine-2-yl]nicotinate was prepared according to general method A from 4-(4-methoxyphenyl)pyridine-2,3-diamine (100 mg, 0.46 mmol), 5-(methoxycarbonyl)pyridine-2-carboxylic acid (84 mg, 0.46 mmol), HBTU (174 mg, 0.46 mmol), DIPEA (60 mg, 0.46 mmol). The crude product was taken directly to the next step (110 mg, 67%, 70% purity).

MS (ESI) m/z 360 (M+1).

### Example 23

### 2-{4-[(3,3-Difluoropyrrolidin-1-yl)carbonyl]phenyl}-7-(4-methoxyphenyl)3H-imidazo[4,5-b]pyridine

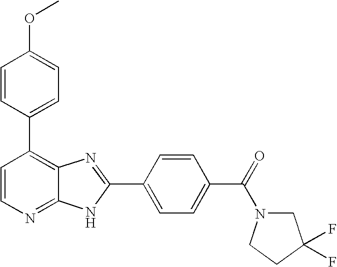

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (obtained from Example 23(a)) (0.085 g, 0.25 mmol) were dissolved in DMF (1 mL) and stirred at r.t. for 90 minutes. 3,3-Difluoropyrrolidine hydrochloride was added (0.053 g, 0.37 mmol) and the mixture was stirred for 2.5 hours. The mixture was filtered and purified by preparative HPLC (MUX), affording 0.029 g (26%) of the title compound.

¹H NMR (DMSO-d₆) δ ppm 13.75 (s, 1 H,), 8.48-8.40 (m, 2 H), 8.40-8.32 (m, 3 H), 7.81-7.72 (m, 2 H), 7.57-7.52 (m, 1 H), 7.20-7.12 (m, 2 H), 4.01-3.89 (m, 2 H), 3.86 (s, 3 H), 3.80-3.71 (m, 2 H), 2.48-2.40 (m, 2 H);

MS (ESI) m/z 435 (M+1).

### Example 23(a)

### 4-[7-(4-Methoxyphenyl)-3H-imidazo[4,5-b]pyridin-2-yl]benzoic acid

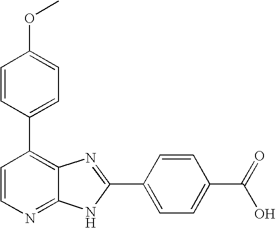

A mixture of methyl 4-[7-(4-methoxyphenyl)-3H-imidazo[4,5-b]pyridin-2-yl]benzoate (1.75 g, 4.87 mmol, obtained from Example 3(a)) and lithium hydroxide (1.23 g, 29.0 mmol) in THF/water (9:1) was heated in microwave reactor at +120° C. for 10 minutes. After cooling to r.t. the mixture was neutralized using 2M HCl (aq.). The precipitate was filtered, washed with water and dried to afford the crude yield of 1.6 g (95%).

¹H NMR (DMSO-d₆) δ ppm 8.51-8.39 (m, 3 H), 8.38-8.24 (m, 2 H), 8.20-8.09 (m, 2 H), 7.61 (d, 1 H), 7.18 (d, 2 H), 3.91 (s, 3 H) MS (ESI) m/z 346 (M+1).

### Example 24

### 7-(4-Methoxyphenyl)-2-(4-{[(3R)-3-methylmorpholin-4-yl]carbonyl}phenyl)-3H-imidazo[4,5-b]pyridine

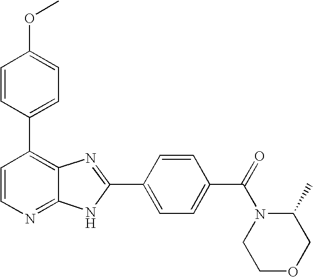

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. (3S)-3-Methylmorpholine (0.037 g, 0.37 mmol) was added and the mixture was stirred for 2.5 hours. The mixture was filtered and purified by preparative HPLC (MUX), affording 0.009 g (8.4%) of the title compound.

¹H NMR (DMSO-d₆) δ ppm 13.72 (br s, 1 H), 8.48-8.40 (m, 2 H), 8.39-8.30 (m, 3 H), 7.62-7.57 (m, 2 H), 7.56-7.51 (m, 1 H), 7.15 (d, 2 H), 3.86 (s, 3 H), 3.86-3.79 (m, 2 H), 3.66-3.53 (m, 2 H), 3.49-3.36 (m, 2 H), 3.01-2.97 (m, 1 H), 1.30-1.25 (m, 3 H);

MS (ESI) m/z 429 (M+1).

### Example 25

### 2-[4-[(4-Ethylpiperazin-1-yl)carbonyl]phenyl}-7-(4-methoxyphenyl)-3H-imidazo[4,5-b]pyridine

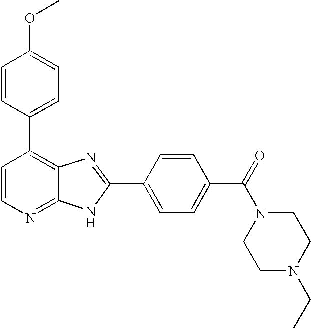

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. 1-Ethylpiperazine (0.042 g, 0.37 mmol) was added and the mixture was stirred for 2.5 hours. The mixture was filtered and purified by preparative HPLC (MUX), affording is 0.024 g (21%) of the title compound.

¹H NMR (DMSO-d₆) δ ppm 13.72 (br s, 1 H), 8.49-8.40 (m, 2 H), 8.38-8.30 (m, 3 H), 7.63-7.56,(m, 2 H), 7.56-7.51 (m, 1 H), 7.15 (d, 2 H), 3.86 (s, 3 H), 3.74-3.53 (m, 2 H), 3.46-3.34 (m, 2 H), 2.46-2.29 (m, 6 H), 1.01 (t, 3 H); MS (ESI) m/z 442 (M+1).

### Example 26

### 4-[7-(4-Methoxyphenyl)-3H-imidazo[4,5-b]pyridin-2-yl]-N-(2-piperidin-1-ylethyl)benzamide

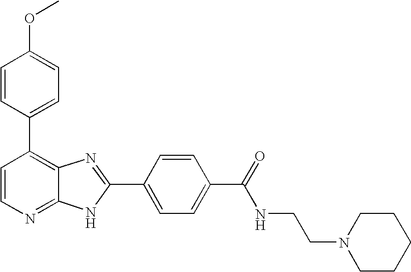

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. 2-Piperidine-1-ylethanamine (0.047 g, 0.37 mmol) was added and the mixture was stirred for 2.5 hours. The mixture was filtered and purified by preparative HPLC (MUX), affording 0.013 g (11%) of the title compound.

¹H NMR (DMSO-d₆) δ ppm 13.72 (br s, 1 H), 8.47-8.40 (m, 2 H), 8.39-8.32 (m, 3 H), 8.05-7.98 (m, 2 H), 7.57-7.50 (m, 1 H), 7.16 (d, 2 H), 3.86 (s, 3 H), 3.46-3.37 (m, 2 H), 2.49-2.44 (m, 2 H), 2.45-2.38 (m, 4 H), 1.56-1.45 (m, 4 H), 1.44-1.33 (m, 2 H);

MS (ESI) m/z 456 (M+1).

### Example 27

### 7-(4-Methoxyphenyl)-2-{4-[(4-methyl-1,4-diazepan-1-yl)carbonyl]phenyl}-3H-imidazo[4,5-b]pyridine

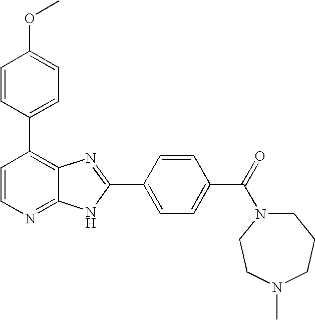

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. Methyl-1,4-diazepane (0.042 g, 0.37 mmol) was added and the mixture was stirred for 2.5 hours. The mixture was filtered and purified by preparative HPLC (MUX), affording 0.025 g (22%) of the title compound.

¹H NMR (DMSO-d₆) δ ppm 13.67 (br s, 1 H), 8.49-8.40 (m, 2 H), 8.39-8.29 (m, 3 H), 7.62-7.56 (m, 2 H), 7.55-7.51 (m, 1 H), 7.19-7.11 (m, 2 H), 3.86 (s, 3 H), 3.71-3.60 (m, 2 H), 3.48-3.36 (m, 2 H), 2.70-2.63 (m, 1 H), 2.61-2.55 (m, 1 H), 2.32-2.28 (m, 1 H), 2.28-2.22 (m, 1 H), 1.92-1.83 (m, 1 H), 1.82-1.71 (m, 1 H);

MS (ESI) m/z 442 (M+1).

### Example 28

### 1-{4-[7-(4-Methoxyphenyl)-3H-imidazo[4,5-b]pyridin-2-yl]benzoyl}-N,N-dimethylpyrrolidin-3-amine

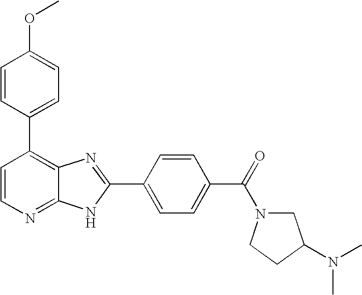

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23 (a)) were dissolved in DMF (1 mL) and stirred at r.t. for 90 minutes. N,N-Dimethylpyrrolidin-3-amine (0.042 g, 0.37 mmol) was added and the mixture was stirred for 2.5 hours. The mixture was then filtered and purified by preparative HPLC (MUX), affording 0.024 g (21%) of the title compound.

¹H NMR (DMSO-d₆) δ ppm 13.72 (br s, 1 H), 8.51-8.39 (m, 2 H), 8.39-8.25 (m, 3 H), 7.78-7.66 (m, 2 H), 7.59-7.49 (m 1 H), 7.21-7.09 (m, 2 H), 3.86 (s, 3 H), 3.81-3.71 (m, 1 H), 3.70-3.60 (m, 1 H), 3.59-3.45 (m, 2 H), 2.80-2.63 (m, I H), 2.20 (s, 3 H), 2.11 (s, 3 H), 2.07-1.98 (m, 1 H), 1.82-1.65 (m, 1 H); MS (ESI) m/z 442 (M+1).

### Example 29

### 2-(4-{[4-(2-Methoxyethyl)piperazin-1-yl]carbonyl}phenyl)-7-(4-methoxyphenyl)-3H-imidazo[4,5-b]pyridine

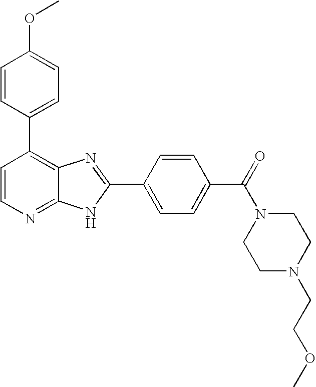

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. 1-(2-Methoxyethylpiperazine (0.053 g, 0.37 mmol) was added and the mixture was stirred for 2.5 hours. The mixture was filtered and purified by preparative HPLC (MUX), affording 0.020 g (17%) of the title compound.

¹H NMR (DMSO-d₆) δ ppm 13.72 (br s, 1 H), 8.48-8.39 (m, 2 H), 8.39-8.29 (m, 3 H), 7.62-7.55 (m, 2 H), 7.55-7.51 (m, 1 H), 7.19-7.11 (m, 2 H), 3.86 (s, 3 H), 3.71-3.56 (m, 2 H),3.48-3.42 (m, 2 H), 3.41-3.33 (m, 2 H), 3.23 (s, 3 H), 2.53-2.38 (m, 6 H);

MS (ESI) m/z 472 (M+1).

### Example 30

### 2-{4-[(4-Isopropylpiperazin-1-yl)carbonyl]phenyl}-7-(4-methoxyphenyl)-3H-imidazo[4,5-b]pyridine

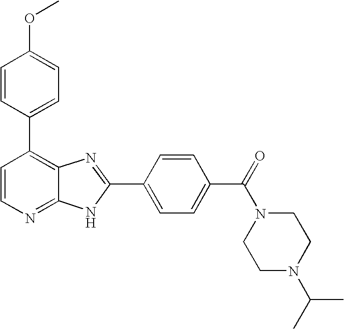

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. 1-Isopropylpiperazine (0.047 g, 0.37 mmol) was added and the mixture was stirred for 2.5 hours. The mixture was filtered and purified by preparative HPLC (MUX), affording 0.019 g (17%) of the title compound.

¹H NMR (DMSO-d₆) δ ppm 13.72 (br s, 1 H), 8.48-8.40 (m, 2 H), 8.40-8.29 (m, 3 H), 7.62-7.56 (m, 2 H), 7.56-7.52 (m, 1 H), 7.18-7.12 (m, 2 H), 3.86 (s, 3 H), 3.71-3.55 (m, 2 H), 3.41-3.33 (m, 2 H), 2.74-2.65 (m, 1 H), 2.48-2.36 (m, 4H), 1.02-0.93 (m, 6 H); MS (ESI) m/z 456 (M+1).

### Example 31

### 2-(4-{[(3S)-3-Fluoropyrrolidin-1-yl]carbonyl}phenyl)-7-(4-methoxyphenyl)-3H-imidazo[4,5-b]pyridine

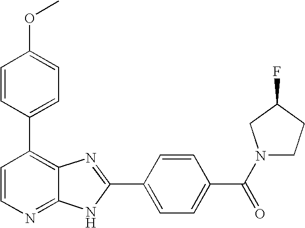

Triethylamine (0.075 g, 0.74 mmol), TSTU (0.093 g, 0.31 mmol) and 4-[7-(4-methoxyphenyl)-3H-imidazo[4,5-b]pyridin-2-yl]benzoic acid (0.085 g, 0.25 mmol, obtained from Example 23(a)) were dissolved in DMF (1 mL) and stirred at r.t. for 90 minutes. (3S)-3-Fluoropyrrolidine hydrochloride (0.046 g, 0.37 mmol) was added and the mixture was stirred for 2.5 hours. The mixture was filtered and purified by preparative HPLC (MUX), affording 0.016 g (16%) of the title compound.

¹H NMR (DMSO-d₆) δ ppm 13.74 (br s, 1 H), 8.47-8.41 (m, 2 H), 8.38-8.31 (m, 3 H), 7.80-7.70 (m, 2 H), 7.56-7.52 (m, 1 H), 7.20-7.11 (m, 2 H), 3.86 (s, 3 H), 3.83-3.51 (m, 4 H), 2.36-2.00 (m, 3 H); MS (ESI) m/z 417 (M+1).

### Example 32

### 1-{4-[7-(4-Methoxyphenyl)-3H-imidazo[4,5-h]pyridin-2-yl]benzoyl}pyrrolidin-3-ol hydrochloride

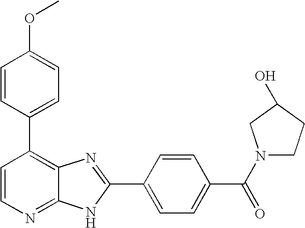

Triethylamine (0.18 g, 1.74 mmol), TSTU (0.22 g, 0.74 mmol) and 4-[7-(4-methoxyphenyl)-3H-imidazo[4,5-b]pyridin-2-yl]benzoic acid (0.20 g, 0.58 mmol, obtained from Example 23(a)) were dissolved in DMF (2 mL) and stirred at r.t. for 10 minutes. Pyrrolidin-3-ol (0.08 g, 0.87 mmol) was added and the mixture was stirred for 10 minutes followed by purification by preparative HPLC. The base was dissolved in THF and hydrochloric acid (1M HCl in diethyl ether) was added until precipitation formed. The solvent was evaporated in vacuo affording 0.042 g (16%) the title compound.

¹H NMR (DMSO-d₆) δ ppm 13.77 (br s, 1 H), 8.53-8.27 (m, 5 H), 7.78-7.66 (m, 2 H), 7.60-7.48 (m, 1 H), 7.23-7.09 (m, 2 H), 5.13-4.90 (m, 1 H), 4.42-4.21 (m, 1 H), 3.86 (s, 3 H), 3.69-3.51 (m, 2 H), 3.50-3.37 (m, 1 H), 3.29-3.19 (m, 1 H), 2.05-1.73 (m, 2 H); MS (ESI) m/z 415 (M+1).

### Example 33

### 7-(3-Fluoro-4-Methoxyphenyl)-2-{4-[(4-methylpiperazin-1-yl)carbonyl]phenyl}-3H-imidazo[4,5-b]pyridine hydrochloride

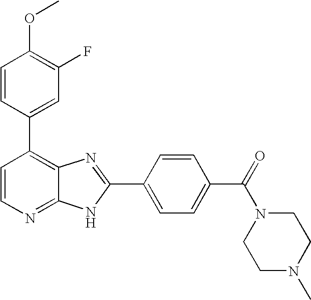

A mixture of the 7-Chloro-2-{4-[(4-methylpiperazin-1-yl)carbonyl]phenyl}-3H-imidazo[4,5-b]pyridine (0.12 g, 0.34 mmol, obtained from Example 5(d)), (3-fluoro-4-methoxyphenyl)boronic acid (0.11 g, 0.67 mmol), PdCl₂(dppf)*DCM (0.014 g, 0.017 mmol) and sodium carbonate (0.20 g, 1.85 mmol) in 2 mL THF/water (9:1) were heated in a microwave reactor at +160° C. for 10 minutes. After cooling to room temperature the mixture was diluted with EtOAc and washed with water. The organic phase was dried (MgSO₄), filtered and evaporated in vacuo. The residue was purified by preparative HPLC, which afforded the product as a base. The base was dissolved in THF and hydrochloric acid (1M HCl in diethyl ether) was added until precipitation formed. The solvent was evaporated in vacuo affording the title compound 0.023 g (12%).

¹H NMR (DMSO-d₆) δ ppm 11.53 (br s, 1 H), 8.59-8.20 (m, 5 H), 7.75-7.50 (m, 3 H), 7.49-7.28 (m, 1 H), 3.95 (s, 3 H), 3.80-3.38 (m, 4 H), 2.43-2.26 (m, 4 H), 2.21 (s, 3 H);

MS (ESI) m/z 446 (M+1).

### Example 34

### 7-(4-Isopropoxyphenyl)-2-{4-[(4-methylpiperazin-1-yl)carbonyl]phenyl}-3H-imidazo[4,5-b]pyridine hydrochloride

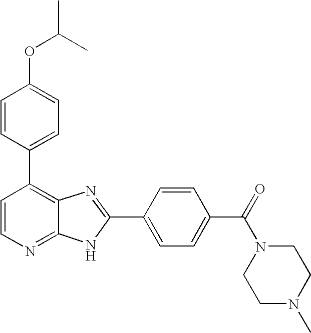

A mixture of the 7-Chloro-2-{4-[(4-methylpiperazin-1-yl)carbonyl]phenyl}-3H-imidazo[4,5-b]pyridine (0.20 g, 0.56 mmol, obtained from Example 5(d)), (4-isopropoxyphenyl)boronic acid (0.20 g, 1.12 mmol), PdCl₂(dppf)*DCM (0.023 g, 0.028 mmol) and sodium carbonate (0.33 g, 3.09 mmol) in 2 mL THF/water (9:1) were heated in a microwave reactor at +160° C. for 10 minutes. After cooling to room temperature the mixture was diluted with EtOAc and washed with water. The organic phase was dried (MgSO₄), filtered and evaporated in vacuo. The residue was purified by preparative HPLC, which afforded the product as a base. The base was dissolved in THF and hydrochloric acid (1M HCl in diethyl ether) was added until precipitation formed. The solvent was evaporated in vacuo affording the title compound 0.041 g (15%).

¹H NMR (DMSO-d₆) δ ppm 13.71 (br s, 1 H), 8.50-8.26 (m, 5 H), 7.64-7.55 (m, 2 H), 7.55-7.48 (m, 1 H), 7.17-7.06 (m, 2 H), 4.80-4.69 (m, 1 H), 3.75-3.56 (m, 2 H), 3.46-3.34 (m, 2 H), 2.45-2.24 (m, 4 H), 2.21 (s, 3 H), 1.33 (d, 6 H); MS (ESI) m/z 456 (M+1).

### Example 35

### 7-(4-Ethoxyphenyl)-2-[4-[(4-methylpiperazin-1-yl)carbonyl]phenyl}-3H-imidazo[4,5-b]pyridine hydrochloride

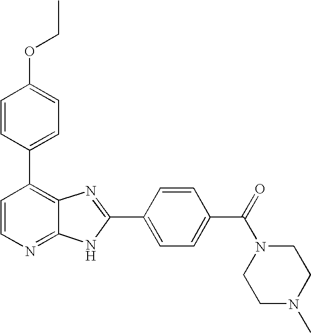

A mixture of the 7-chloro-2-{4-[(4-methylpiperazin-1-yl)carbonyl]phenyl}-3H-imidazo[4,5-b]pyridine (0.20 g, 0.56 mmol, obtained from Example 5(d)), (4-ethoxyphenyl)boronic acid (0.19 g, 1.12 mmol), PdCl₂(dppf)*DCM (0.023 g, 0.028 mmol) and sodium carbonate (0.33 g, 3.09 mmol) in 2 mL THF/water (9:1) were heated in a microwave reactor at +160° C. for 10 minutes. After cooling to room temperature the mixture was diluted with EtOAc and washed with water. The organic phase was dried (MgSO₄), filtered and evaporated in vacuo. The residue was purified by preparative HPLC, which afforded the product as a base. The base was dissolved in THF and hydrochloric acid (1M HCl in diethyl ether) was added until precipitation formed. The solvent was evaporated in vacuo affording the title compound 0.078 g (29%).

¹H NMR (DMSO-d₆) δ ppm 13.73 (br s, 1 H), 8.53-8.23 (m, 5 H), 7.66-7.45 (m, 3 H), 7.24-7.03 (m, 2 H), 4.14 (q, 2 H), 3.75-3.55 (m, 2 H), 3.48-3.33 (m, 2 H), 2.45-2.24 (m, 4 H), 2.21 (s, 3 H), 1.38 (t, 3 H); MS (ESI) m/z 441 (M+1).

### Example 36

### 7-(4-Methoxy-2-methylphenyl)-2-{4-[(4-methylpiperazin-1-yl)carbonyl]phenyl}-3H-imidazo[4,5-b]pyridine hydrochloride

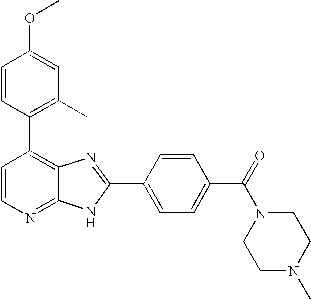

A mixture of the 7-chloro-2-{4-[(4-methylpiperazin-1-yl)carbonyl]phenyl}-3H-imidazo[4,5-b]pyridine (0.20 g, 0.56 mmol, obtained from Example 5(d)), (4-methoxy-2-methylphenyl)boronic acid (0.19 g, 1.12 mmol), PdCl₂(dppf)*DCM (0.023 g, 0.028 mmol) and sodium carbonate (0.33 g, 3.09 mmol) in 2 mL THF/water (9:1) were heated in a microwave reactor at +160° C. for 10 minutes. After cooling to room temperature the mixture was diluted with EtOAc and washed with water. The organic phase was dried (MgSO₄), filtered and evaporated in vacuo. The residue was purified by preparative HPLC, which afforded the product as a base. The base was dissolved in THF and hydrochloric acid (1M HCl in diethyl ether) was added until precipitation formed. The solvent was evaporated in vacuo affording the title compound 0.090 g (34%).

¹H NMR (DMSO-d₆) δ ppm 13.69 (br s, 1 H), 8.46-8.17 (m, 3 H), 7.58-7.48 (m, 2 H), 7.42-7.29 (m, 1 H), 7.18-7.07 (m, 1 H), 7.02-6.87 (m, 2 H), 3.83 (s, 3 H), 3.71-3.53 (m, 2 H), 3.43-3.34 (m, 2 H), 2.43-2.22 (m, 7 H), 2.20 (s, 3 H); MS (ESI) m/z 441 (M+1).

### Example 37

### 7-(4-Methoxyphenyl)-2-{2-[(4-methylpiperazin-1-yl)carbonyl]pyridin-4-yl}-3H-imidazo[4,5-b]pyridine

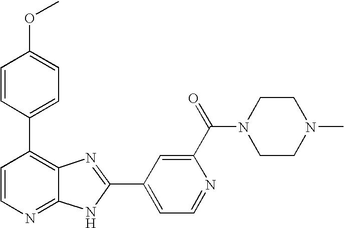

Methyl 4-[7-(4-methoxyphenyl)-3H-imidazo[4,5-b]pyridin-2-yl]pyridine-2-carboxylate (obtained from Example 37(a)) (0.25 g, 1.16 mmol) was mixed with 1-methylpiperazine (2 mL) and heated in a microwave reactor at +200° C. for 15 minutes. Water (10 mL) was added, the product precipitated and was filtered. The crude product was purified by preparative HPLC, affording 30 mg of the title compound (6%).

¹H NMR (DMSO-d₆) δ ppm 13.87 (br s, 1 H), 8.89-8.80 (m, 1 H), 8.47-8.29 (m, 4 H), 7.58-7.51 (m, 2 H), 7.20-7.11 (m, 2 H), 3.86 (s, 3 H), 3.74-3.64 (m, 2 H), 3.40-3.33 (m, 2 H), 2.46-2.38 (m, 2 H), 2.36-2.26 (m, 2 H), 2.22 (s, 3 H); MS (ESI) m/z 429 (M+1).

### Example 37(a)

### Methyl 4-[7-(4-methoxyphenyl)-3H-imidazo[4,5-b]pyridin-2-yl]pyridine-2-carboxylate

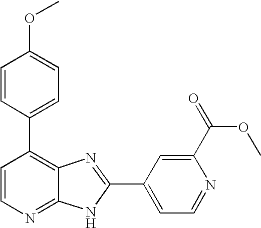

(i-Pr)₂EtN (0.38 g, 2.90 mmol) was added to a suspension of 4-(4-methoxyphenyl)pyridine-2,3-diamine (obtained from Example 1(c))(0.25 g, 1.16 mmol), methyl 4-acetylpyridine-2-carboxylate (Wang et al. J. Am. Chem. Soc. 2003, 125, 296-304) (0.27 g, 1.39 mmol) and HBTU (0.53 g, 1.39 mmol) in acetonitrile (5 mL), and stirred at over night. The mixture was diluted with EtOAc and washed with water. The organic phase was dried (MgSO₄), filtered and evaporated in vacuo. The solid was mixed with HOAc (3 mL) and heated in a microwave reactor at +120° C. for 15 minutes. The solvent was removed in vacuo, added water (30 mL) and filtered the brown solid affording a crude yield of 0.25 g (21%). The crude product was used without purification in the next step.

MS (ESI) m/z 361 (M+1).

### Example 38

### 7-(4-Methoxyphenyl)-2-{5-[(4-methylpiperazin-1-yl)carbonyl]pyridin-3-yl}-3H-imidazo[4,5-b]pyridine

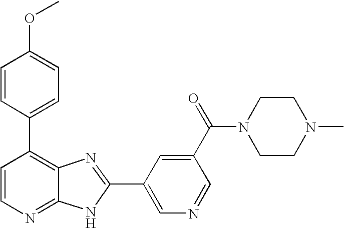

(i-Pr)₂EtN (0.11 g, 0.87 mmol) was added to a suspension of 5-[7-(4-methoxyphenyl)-3H-imidazo[4,5-b]pyridin-2-yl]nicotinic acid (obtained from Example 38(a)) (0.15 g, 0.43 mmol), 1-methylpiperazine (0.052 g, 0.52 mmol) and HBTU (0.16 g, 0.43 mmol) in acetonitrile (5 mL), and stirred over night. The solvent was removed in vacuo, and the residue was diluted with EtOAc and washed with water. The organic phase was dried (MgSO₄), filtered and evaporated in vacuo. The crude product was purified by preparative HPLC, affording 13 mg of the title compound (7%).

¹H NMR (DMSO-d₆) δ ppm 13.90 (br s, 1 H), 9.52-9.46 (m, 1 H), 8.76-8.72 (m, 1 H), 8.62-8.57 (m, 1 H), 8.49-8.41 (m, 2 H), 8.41-8.35 (m, 1 H), 7.62-7.54 (m, 1 H), 7.20-7.11 (m, 2 H), 3.86 (s, 3 H), 3.75-3.65 (m, 2 H), 3.48-3.37 (m, 2 H), 2.46-2.26 (m, 4 H), 2.22 (s, 3 H); MS (ESI) m/z 429 (M+1).

### Example 38(a)

### 5-[7-(4-Methoxyphenyl)-3H-imidazo[4,5-b]pyridin-2-yl]nicotinic acid

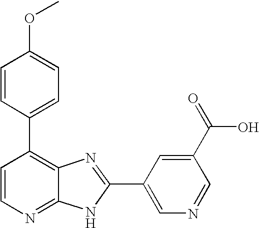

(i-Pr)₂EtN (0.45 g, 3.48 mmol) was added to a suspension of 4-(4-methoxyphenyl)pyridine-2,3-diamine (obtained from Example 1(c)) (0.30 g, 1.39 mmol), pyridine-3,5-dicarboxylic acid (0.23 g, 1.39 mmol) and HBTU (0.53 g, 1.39 mmol) in acetonitrile (5 mL), and refluxed over night. The solvent was removed in vacuo, and the residue was diluted with EtOAc and washed with water. The organic phase was dried (MgSO₄), filtered and evaporated in vacuo. The solid was mixed with HOAc (3 mL) and heated in a microwave reactor at +120° C. for 15 minutes. The solvent was removed in vacuo, added water (30 mL) and filtered the brown solid affording a crude yield of 0.25 g (21%). The crude product was used without purification in the next step.

MS (ESI) m/z 361 (M+1).

### Example 39

### 7-(2-Methoxyphenyl)-2-[4-(morpholin-4-ylcarbonyl)phenyl]-3H-imidazo[4,5-b]pyridine hydrochloride

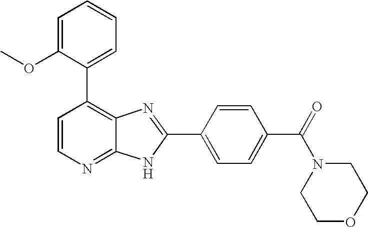

The title compound was prepared in accordance with the general method C using 7-chloro-2-[4-(morpholin-4-ylcarbonyl)phenyl]-3H-imidazo[4,5-b]pyridine (obtained from Example 10(a)) (0.182 g, 0.531 mmol), (2,4-dimethoxyphenyl)boronic acid (0.162 g, 1.06 mmol), PdCl₂(dppf)*DCM (0.022 g, 0.027 mmol) and sodium carbonate (0.169 g, 1.6 mmol), affording 0.021 g (9%) of the title compound.

MS (ESI) m/z 415 (M+1); RT (HPLC) 7.94 min.

### Example 40

### N-(3-Methoxypropyl)-4-(7-pyridin-4-yl-3H-imidazo[4,5-b]pyridin-2-yl)benzamide hydrochloride

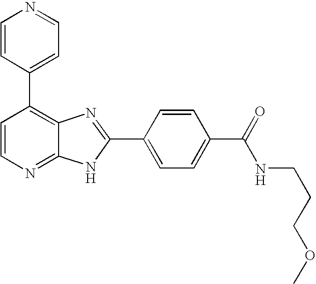

The title compound was prepared in accordance with the general method C using 7-iodo-2-[4-(3-methoxy propyl-4-ylcarbonyl)phenyl]-3H-imidazo[4,5-b]pyridine (obtained from Example 40(b))(0.040 g, 0.092 mmol), 4-pyridylboronic acid (0.038 g, 0.183 mmol), PdCl₂(dppf)*DCM (0.008 g, 0.0092 mmol) and sodium carbonate (0.049 g, 0,46 mmol), affording 0.013 g (31%) of the title compound.

¹H NMR (DMSO-d₆) δ ppm; 9.02 (d, 2 H), 8.90 (s, 2 H), 8.64 (t, 1 H), 8.57 (d, 1 H), 8.41 (d, 2 H), 8.06 (d, 2 H), 7.89 (d, 1 H), 3.26 (s, 3 H), several peaks obscured; MS (APPI) m/z 388 (M+1).

### Example 40(a)

### 4-(7-iodo-3H-imidazo[4,5-b]pyridin-2-yl)benzoic acid

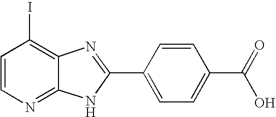

A mixture of methyl 4-(7-iodo-3H-imidazo[4,5-b]pyridin-2-yl)benzoate (obtained from Example 14 (a)) (0.20 g, 0.527 mmol) and lithium hydroxide (0.076 g, 3.17 mmol) in THF/water (9:1) was heated in microwave reactor at +120° C. for 10 minutes. After cooling to r.t. the mixture was neutralized using 2M HCl (aq.). The precipitate was filtered, washed with water and dried to afford the crude yield of 0.158 g (82%).

MS (APPI) m/z 366 (M+1); RT (HPLC) 2.23 min

### Example 40(b)

### 7-Iodo-2-[4-(3-methoxy propyl-4-ylcarbonyl)phenyl]-3H-imidazo[4,5-b]pyridine

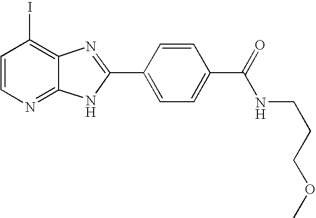

The title compound was prepared in accordance with the general method B using 4-(7-iodo-3H-imidazo[4,5-b]pyridin-2-yl)benzoic acid (obtained from Example 40(a)) (0.060 g, 0.164 mmol), TSTU (0.059 g, 0.197 mmol), triethylamine (0.050 g, 0.493 mmol) and 3-Methoxypropylamine (0.022 g, 0.247 mmol), affording 0.045 g (63%) of the title compound.

MS (APPI) m/z 437 (M+1); RT (HPLC) 2.97 min

### Example 41

### 2-{4-[(4-Methylpiperazin-1-yl)carbonyl]phenyl-7-pyridin-4-yl-3H-imidazo[4,5-b]pyridine hydrochloride

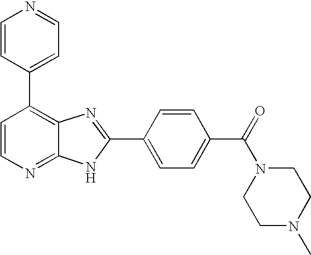

The title compound was prepared in accordance with the general method C using 7-chloro-2-{4-[(4-methylpiperazin-1-yl)carbonyl]phenyl}-3H-imidazo[4,5-b]pyridine (obtained from Example 5(d)) (0.100 g, 0.282 mmol), 4-pyridylboronic acid (0.069 g, 0.563 mmol), PdCl₂(dppf)*DCM (0.011 g, 0.014 mmol) and sodium carbonate (0.149 g, 1.41 mmol), affording 0.016 g (12%) of the title compound.

¹H NMR (DMSO-d₆) δ ppm; 9.01 (d, 2 H), 8.84-8.92 (m, 2 H), 8.56 (d, 1 H), 8.43 (d, 2 H), 7.89 (d, 1 H), 7.70 (d, 2 H), 2.79 (s, 3 H); MS (APPI) in/z 399 (M+1).

### Example 42

### 2-{4-[(4-Methylpiperazin-1-yl)methyl]phenyl}-7-pyridin-4-yl-3H-imidazo[4,5-b]pyridine hydrochloride

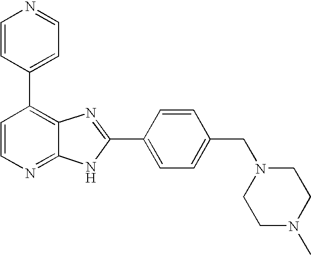

The title compound was prepared in accordance with the general method C using 7-chloro-2-{4-[(4-methylpiperazin-1-yl)methyl]phenyl}-3H- imidazo[4,5-b]pyridine (obtained from Example 42(a)) (0.050 g, 0.146 mmol), 4-pyridylboronic acid (0.036 g, 0.292 mmol), PdCl₂(dppf)*DCM (0.006g, 0.007 mmol) and sodium carbonate (0.078 g, 0.73 mmol), affording 0.026 g (36%) of the title compound.

¹H NMR (D₂O) δ ppm; 8.92 (d, 2 H), 8.65 (d, 2 H), 8.45 (d, 1 H), 8.11 (d, 2 H), 7.60-7.75 (m, 3 H), 4.55 (s, 2 H), 3.38-4.05 (m, 8 H), 2.85-3.13 (m, 3 H); MS (ESI) m/z 385 (M+1).

### Example 42(a)

### 7-Chloro-2-{4-[(4-methylpiperazin-1-yl)methyl]phenyl}-3H-imidazo[4,5-b]pyridine

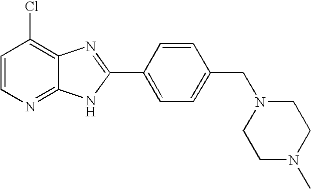

Borane-THF complex (1M, 3 mL) was added to the crude intermediate 7-chloro-2-{4-[(4-methylpiperazin-1-yl)carbonyl]phenyl}-3H-imidazo[4,5-b]pyridine (obtained from Example 5(d)) at r.t. After stirring at r.t. for 45 minutes, MeOH (1 mL) was added dropwise to the reaction mixture and the mixture was stirred for 2 h at r.t.. The solvent was evaporated in vacuo, and co-evaporated with MeOH, affording the title compound in 0.79 g (63%). The title compound was used in the next step without frrther purification.

MS (APPI) m/z 342/344 Cl isotope (M+1).

### Example 43

### 4-(2-{4-[(4-Methylpiperazin-1-yl)methyl]phenyl}-3H-imidazo[4,5-b]pyridin-7-yl)benzamide hydrochloride

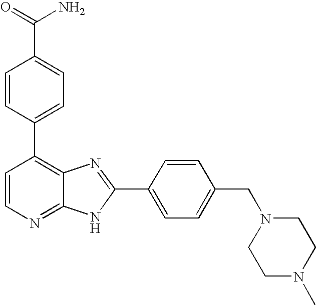

The title compound was prepared in accordance with the general method C using 7-chloro-2-{4-[(4-methylpiperazin-1-yl)methyl]phenyl}-3H-imidazo[4,5-b]pyridine (0.050 g, mmol, obtained from Example 42(a)), (4-carbamoylphenyl)boronic acid (0.048 g, 0.292 mmol), PdCl₂(dppf)*DCM (0.006 g, 0.007 mmol) and sodium carbonate (0.078 g, 0.73 mmol), affording 0.043 g (55%) of the title compound.

¹H NMR (DMSO-d₆) δ ppm; 8.45 (d, 1 H), 8.41 (d, 2 H), 8.35 (d, 2 H), 8.08 (d, 2 H), 7.79 (d, 2 H), 7.64 (d, 1 H), 2.82 (br s, 3 H); MS (APPI) m/z 427 (M+1).

### Example 44

### 7-(4-Methoxyphenyl)-2-{4-[(4-methylpiperazin-1-yl)methyl]phenyl}-3H-imidazo[4,5-b]pyridine hydrochloride

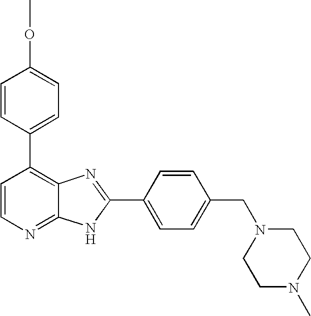

The title compound was prepared in accordance with the general method C using 7-chloro-2-{4-[(4-methylpiperazin-1-yl)methyl]phenyl}-3H-imidazo[4,5-b]pyridine (0.050 g, 0.146 mmol, obtained from Example 42(a)), 4-methoxyphenylboronic acid (0.044 g, 0.292 mmol), PdCl₂(dppf)*DCM (0.006 g, 0.007 mmol) and sodium carbonate (0.078 g, 0.73 mmol), affording 0.021 g (27%) of the title compound.

MS (APPI) m/z 414 (M+1).

### Example 45

### 7-(4-Ethoxyphenyl)-2-{4-[(4-methylpiperazin-1-yl)methyl]phenyl)-3H-imidazo[4,5-b]pyridine hydrochloride

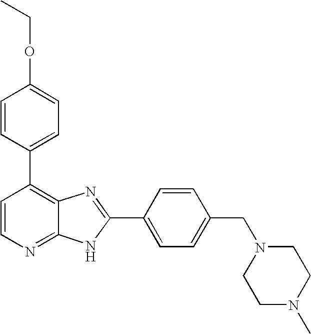

The title compound was prepared in accordance with the general method C using 7-chloro-2-{4-[(4-methylpiperazin-1-yl)methyl]phenyl}-3H-imidazo[4,5-b]pyridine (0.050 g, 0.146 mmol, obtained from Example 42(a)), 4-ethoxyphenylboronic acid (0.049 g, 0.292 mmol), PdCl₂(dppf)*DCM (0.006 g, 0.007 mmol) and sodium carbonate (0.078 g, 0.73 mmol), affording 0.011 g (15%) of the title compound.

¹H NMR (D₂O) δ ppm; 8.46 (d, 1 H), 8.18 (d, 2 H), 7.85 (d, 2 H), 7.62-7.74 (m, 3 H), 7.15 (d, 2 H), 4.37 (s, 2 H), 4.19 (q, 2 H), 3.29-3.75 (m, 8 H), 2.98 (s, 3 H), 1.42 (t, J=7.07 Hz, 3 H,); MS (API) m/z 428 (M+1).

### Example 46

### (4-{2-[4-(Morpholin-4-ylmethyl)phenyl]-3H-imidazo[4,5-b]pyridin-7-yl}phenyl)methanol hydrochloride

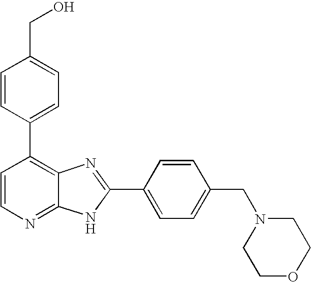

The title compound was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), 4-(hydroxymethyl)phenylboronic acid (0.093 g, 0.610 mmol), PdCl₂(dppf)*DCM (0.025 g, 0.030 mmol) and sodium carbonate (0.194 g, 1.83 mmol), affording 0.039 g (27%) of the title compound.

¹H NMR (DMSO-d₆) δ ppm; 10.89 (s, 1 H), 8.42 (d, 1 H), 8.37 (d, 2 H), 8.29 (d, 2 H), 7.79 (d, 2 H), 7.57 (d, 1 H), 7.53 (d, 2 H), 4.61 (s, 2 H), 4.43 (s, 2 H), 3.88-4.02 (m, 2 H), 3.70-3.82 (m, 3 H), 3.24-3.31 (m, 2 H), 3.08-3.21 (m, 2 H); MS (APPI) m/z 401 (M+1).

### Example 46(a)

### 7-Chloro-2-[4-(morpholin-4-ylmethyl)phenyl]-3H-imidazo[4,5-b]pyridine

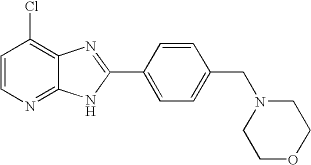

Borane-THF complex (1M, 20 mL) was added to 7-chloro-2-[4-(morpholin-4-ylcarbonyl)phenyl]-3H-imidazo[4,5-b]pyridine (1.7 g, 4.9 mmol, obtained from Example 10(a)) at r.t.. After stirring at r.t. for 45 minutes, MeOH (200 mL) was added dropwise to the reaction mixture and the mixture was stirred for 2 h at r.t.. The solvent was evaporated in vacuo, affording a crude of the title compound in 1.0 g (67%) yield. The crude product was used in the next step without further purification.

MS (APPI) m/z 329 (M+1).

### Example 47

### N-Methyl-4-{2-[4-(morpholin-4-ylmethyl)phenyl]-3H-imidazo[4,5-b]pyridin-7-yl}benzamide hydrochloride

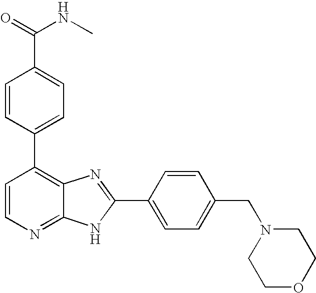

The title compound was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), 4-(N-methylaminocarbonyl)phenyl boronic acid (0.109 g, 0.610 mmol), PdCl₂(dppf)*DCM (0.025 g, 0.030 mmol) and sodium carbonate (0.194 g, 1.83 mmol), affording 0.005 g (3%) of the title compound.

¹H NMR (DMSO-d₆) δ ppm; 10.84 (s, 1 H), 8.53-8.61 (m, 1 H), 8.41-8.48 (m, 3 H), 8.37 (d, 2 H), 8.03 (d, 2 H), 7.79 (d, 2 H), 7.63 (d, 1 H), 4.40-4.46 (m, 2 H), 3.96 (d, 2 H), 3.76 (t, 2 H), 3.29 (d, 2 H), 3.09-3.22 (m, 2 H), 2.84 (d, 3 H); MS (ESI) m/z 426 (M−1).

### Example 48

### 2-[4-(Morpholin-4-ylmethyl)phenyl]-7-[4-(pyrrolidin-1-ylcarbonyl)phenyl]-3H-imidazo[4,5-b]pyridine hydrochloride

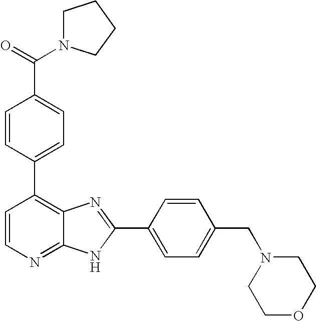

The title compound was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), 4-(pyrrolidine-1-carbonyl)phenylboronic acid (0.133 g, 0.610 mmol), PdCl₂(dppf)*DCM (0.025 g, 0.030 mmol) and sodium carbonate (0.194 g, 1.83 mmol), affording 0.042 g (25%) of the title compound.

¹H NMR (DMSO-d₆) δ ppm; 11.09-11.37 (m, 1 H), 8.45 (d, 1 H), 8.38 (t, 4 H), 7.82 (d, 2 H), 7.72 (d, 2 H), 7.62 (d, 1 H), 4.42 (s, 2 H), 3.91-4.01 (m, 2 H), 3.72-3.87 (m, 2 H), 3.22-3.32 (m, 2 H), 3.02-3.19 (m, 2 H), 1.76-2.01 (m4 H); MS (ESI) m/z 466 (M−1).

### Example 49

### 4-{2-[4-(Morpholin-4-ylmethyl)phenyl]-3H-imidazo[4,5-b]pyridin-7-yl}benzamide hydrochloride

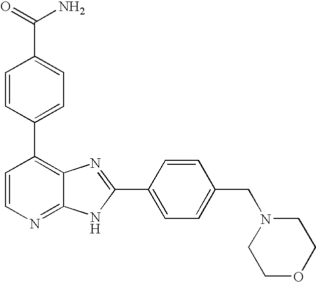

The title compound was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), (4-aminocarbonylphenyl)boronic acid (0.101 g, 0.610 mmol), PdCl₂(dppf)*DCM (0.025 g, 0.030 mmol) and sodium carbonate (0.194 g, 1.83 mmol), affording 0.031 g (21%) of the title compound.

¹H NMR (DMSO-d₆) δ ppm; 10.68 (s, 1 H), 8.40-8.47 (m, 3 H), 8.37 (d, 2 H), 8.07 (d, 3 H), 7.78 (d, 2 H), 7.63 (d, 1 H), 7.46 (s, 1 H), 4.37-4.50 (m, 2 H), 3.90-4.02 (m, 2 H), 3.71-3.78 (m, 2 H), 3.25-3.34 (m, 2 H), 3.07-3.21 (m, 2 H); MS (APPI) m/z 414 (M+1).

### Example 50

### (4-{2-[4-(Morpholin-4-ylmethyl)phenyl]-3H-imidazo[4,5-b]pyridin-7-yl}phenyl)acetonitrile hydrochloride

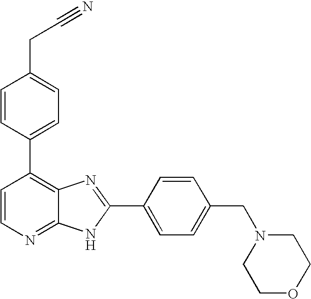

The title compound was prepared in accordance with the general method C using 7-chloro-2-[4-(morpholin-4-ylmethyl)phenyl]-3H-imidazo[4,5-b]pyridine (0.100 g, 0.152 mmol, obtained from Example 46(a)), (4-cyanomethylphenyl)boronic acid (0.049 g, 0.305 mmol), PdCl₂(dppf)*DCM (0.012 g, 0.015 mmol) and sodium carbonate (0.194 g, 1.83 mmol), affording 0.006 g (8%) of the title compound.

¹H NMR (DMSO-d₆) δ ppm; 8.43 (d, 1 H), 8.37 (d, 4 H), 7.78 (d, 2 H), 7.54-7.59 (m, 3 H), 4.43 (s, 2 H), 4.16 (s, 2 H), 3.96 (d, 2 H), 3.75 (t, 2 H), 3.29 (d, 2 H), 3.08-3.20 (m, 2 H); MS (AP) m/z 410 (M+1).

### Example 51

### 4-{2-[4-(Morpholin-4-ylmethyl)phenyl]-3H-imidazo[4,5-b]pyridin-7-yl}benzoic acid hydrochloride

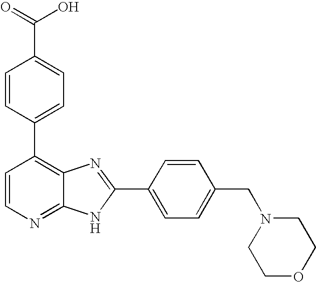

The crude intermediate methyl 4-{2-[4-(morpholin-4-ylmethyl)phenyl]-3H-imidazo[4,5-b]pyridin-7-yl}benzoate (obtained from Example 51(a)) (0.050g, 0.117 mmol) was mixed with LiOH (0.025 gr, 0.595 mmol) in THF/ H₂O 9:1 (4 mL) and the mixture was heated in a microwave reactor at +110° C. for 10 min. The residue was purified by preparative HPLC, which afforded the freebase of the title compound (0.037 g, 79%). The base (0.010 g, 0.024 mmol) was dissolved in CH₂Cl₂/MeOH (9:1) and hydrochloric acid (1M HCl in diethyl ether) was added until precipitation formed. The solid hydrochloride salt was collected by filtration and dried, affording 0.010 g, (85%) of the title compound.

¹H NMR (DMSO-d₆) δ ppm; 8.47 (d, 2 H), 8.40 (d, 1 H), 8.25 (d, 2 H), 8.11 (d, 2 H), 7.60 (d, 1 H), 7.52 (d, 2 H), 3.58-3.62 (m, 4 H), 3.56 (s, 3 H), 2.36-2.43 (m, 4 H); MS (APPI) m/z 426 (M+1).

### Example 51(a)

### Methyl 4-[2-[4-(morpholin-4-ylmethyl)phenyl]-3H-imidazo[4,5-b]pyridin-7-yl}benzoate

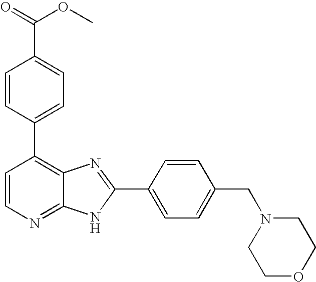

Methyl 4-{2-[4-(morpholin-4-ylmethyl)phenyl]-3H-imidazo[4,5-b]pyridin-7-yl}benzoate was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), 4-methoxycarbonylphenylboronic acid (0.110 g, 0.610 mmol), PdCl₂(dppf)*DCM (0.025 g, 0.030 mmol) and sodium carbonate (0.194 g, 1.83 mmol), affording 0.052 g (27%) after precipitation from water.

### Example 52

### 4,4′-(3H-Imidazo[4,5-b]pyridine-2,7-diyl)dibenzoic acid

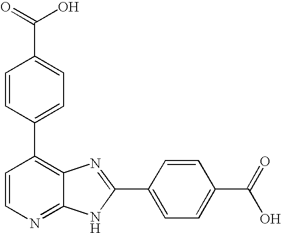

4,4′-(3H-Imidazo[4,5-b]pyridine-2,7-diyl)dibenzoic acid was prepared according to the procedure described for 4-{2-[4-(Morpholin-4-ylmethyl)phenyl]-3H-imidazo[4,5-b]pyridin-7-yl}benzoic acid (Example 51)using 7-chloro-2-[4-(morpholin-4-ylcarbonyl)phenyl]-3H-imidazo[4,5-b]pyridine (obtained from Example 10(a)) (0.050 g, 0.146 mmol), 4-methoxycarbonylphenylboronic acid (0.0.053 g, 0.292 mmol), PdCl₂(dppf)*DCM (0.012 g, 0.015 mmol) and sodium carbonate (0.077 g, 0.731 mmol). The intermediate was then hydrolysed without further purification, using LiOH (0.025 g, 0.595 mmol) in THF/H₂O 9:1 (4 mL), to give the title compound (0.002 g).

¹H NMR (DMSO-d₆) δ ppm; 8.38-8.51 (m, 5 H), 8.10-8.19 (m, 4 H), 7.64 (d, 1 H)); RT (HPLC) 2.437 min,

### Example 53

### 7-[4-(Azetidin-1-ylcarbonyl)phenyl]-2-[4-(morpholin-4-ylmethyl)phenyl]-3H-imidazo[4,5-b]pyridine hydrochloride

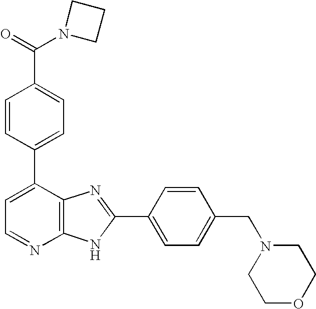

The title compound was prepared using the procedure described in Example 23 using 4-{2-[4-(morpholin-4-ylmethyl)phenyl]-3H-imidazo[4,5-b]pyridin-7-yl}benzoic acid (0.025 g, 0.060 mmol, obtained from Example 51), TSTU (0.020 g, 0.066 mmol), azetidine (0.004 g, 0.072 mmol) and triethylamine (0.018 g, 0.18 mmol) to afford 0.020 g (62%) of the freebase of the title compound. The hydrochloride was prepared according to the method described within general method E.

¹H NMR (DMSO-d₆) δ ppm; 11.05 (s, 1 H), 8.41-8.47 (m, 3 H), 8.37 (d, 1 H), 7.78-7.86 (m, 4 H), 7.62 (d, 1 H), 4.34-4.50 (m, 3 H), 4.09 (s, 2 H), 3.89-3.99 (m, 2 H), 3.68-3.85 (m, 2 H), 2.97-3.34 (m, 4 H), 2.23-2.36 (m, 2 H); MS (ESI) m/z 452 (M−1).

### Example 54

### 1-(4-{2-[4-(Morpholin-4-ylmethyl)phenyl]-3H-imidazo[4,5-b]pyridin-7-yl}phenyl)ethanone hydrochloride

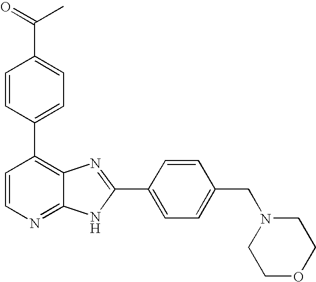

The title compound was prepared in accordance with the general method C using 7-chloro-2-[4-(morpholin-4-ylmethyl)phenyl]-3H-imidazo[4,5-b]pyridine (0.100 g, 0.305 mmol, obtained from Example 46(a)), 4-(hydroxymethyl)phenylboronic acid (0.093 g, 0.610 mmol), PdCl₂(dppf)*DCM (0.025 g, 0.030 mmol) and sodium carbonate (0.194 g, 1.83 mmol), affording 0.039 g (27%) of the title compound.

¹H NMR (DMSO-d₆) δ ppm; 11.10 (s, 1 H), 8.48-8.56 (m, 2 H), 8.46 (d, 1 H), 8.37 (d, 2 H), 8.16 (d, 2 H), 7.81 (d, 1 H), 7.65 (d, 1 H), 4.32-4.52 (m, 2 H), 3.89-4.02 (m, 2 H), 3.79 (t, 2 H), 3.23-3.44 (m, 2 H), 3.07-3.18 (m, 2 H), 2.67 (s, 3 H); MS (APPI) m/z 314 (M+1).

### Example 55

### 7-(4-Methoxyphenyl)-2-[3-(morpholin-4-ylmethyl)phenyl]-3H-imidazo[4,5-b]pyridine hydrochloride

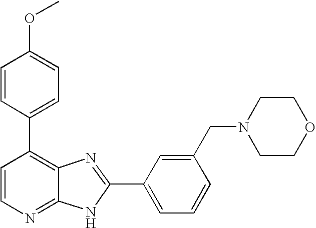

The title compound was prepared in accordance with the general method D using 4-(4-methoxyphenyl)pyridine-2,3-diamine (obtained from Example 1(c)) (50 mg, 0.232 mmol) and 3-morpholin-4-ylmethyl-benzoic acid (56 mg, 0.255 mmol), affording 0.041 g (45%) of the title compound.

¹H NMR (DMSO-d₆) δ ppm; 11.21 (s, 1 H), 8.49-8.53 (m, 1 H), 8.44 (d, 1 H), 8.36 (d, 1 H), 8.29 (d, 2 H), 7.86 (d, 1 H), 7.70 (t, 1 H), 7.59 (d, 1 H), 7.17 (d, 3 H), 4.47 (s, 2 H), 3.92-4.01 (m, 2 H), 3.88 (s, 3 H), 3.79 (d, 2 H), 3.26-3.34 (m, 2 H), 3.09-3.21 (m, 2 H);

MS (AP) in/z 401 (M+1).

### Example 56

### 7-(4-Methoxyphenyl)-2-[3-[(4-methylpiperazin-1-yl)methyl]phenyl)-3H-imidazo[4,5-b]pyridine hydrochloride

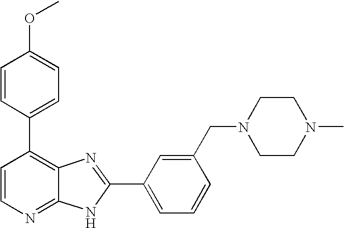

The title compound was prepared in accordance with the general method D using 4-(4-methoxyphenyl)pyridine-2,3-diamine (obtained from Example 1(c)) (50 mg, 0.232 mmol) and 3-(4-methyl-piperazin-1-ylmethyl)-benzoic acid (60 mg, 0.255 mmol), affording 0.053 g (43%) of the title compound.

¹H NMR (DMSO-d₆) δ ppm; 11.58 (s, 1 H), 8.39-8.51 (m, 2 H), 8.26-8.37 (m, 3 H), 7.75-7.89 (m, 1 H), 7.68 (t, 1 H), 7.58 (d, 1 H), 7.17 (d, 2 H), 4.20-4.62 (m, 2 H), 3.87 (s, 3 H), 3.21-3.74 (m, 56 H), 2.81 (s, 3 H); MS (AP) m/z 414 (M+1).

### Example 57

### 7-(4-Methoxyphenyl)-2-phenyl-3H-imidazo[4,5-b]pyridine

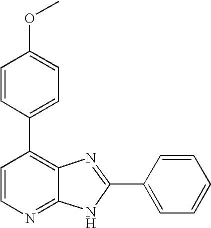

The title compound was prepared in accordance with the general method D, except that the salt was not prepared, using 4-(4-methoxyphenyl)pyridine-2,3-diamine (obtained from Example 1(c)) (50 mg, 0.232 mmol) and benzoic acid (31 mg, 0.255 mmol), affording 0.021 g (9%) of the title compound.

¹H NMR (DMSO-d₆) δ ppm; 13.59 (s, 1 H), 8.44 (d, 2 H), 8.23-8.35 (m, 3 H), 7.47-7.65 (m, 4 H), 7.15 (d, 2 H), 3.86 (s, 3 H); MS (ESI) m/z 300 (M−1).

### Example 58

### 7-(4-Methoxyphenyl)-2-[3-(methylsulfonyl)phenyl]-3H-imidazo[4,5-b]pyridine

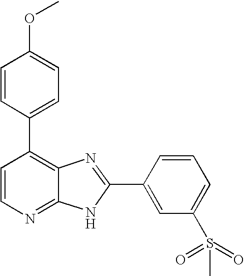

The title compound was prepared in accordance with the general method D, except that the salt was not prepared, using 4-(4-methoxyphenyl)pyridine-2,3-diamine (obtained from Example 1(c)) (50 mg, 0.232 mmol) and 3-methylsulphonyl benzoic acid (51 mg, 0.255 mmol), affording 0.015 g (17%) of the title compound.

¹H NMR (DMSO-d₆) δ ppm; 13.87 (s, 1 H), 8.80 (s, 1 H), 8.56-8.65 (m, 1 H), 8.32-8.47 (m, 3 H), 8.03-8.13 (m, 1 H), 7.88 (t, 1 H), 7.52-7.59 (m, 1 H), 7.16 (d, 2 H), 3.87 (s, 3 H), 3.33 (s, 3 H); MS (ESI) m/z 378 (M−1).

### Example 59

### 7-(4-Methoxyphenyl)-2-[4-(methylsulfonyl)phenyl]-3H-imidazo[4,5-b]pyridine

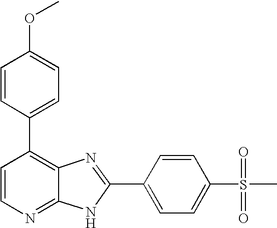

The title compound was prepared in accordance with the general method D, except that the salt was not prepared, using 4-(4-methoxyphenyl)pyridine-2,3-diamine (obtained from Example 1(c)) (50 mg, 0.232 mmol) and 4-methylsulphonyl benzoic acid (51 mg, 0.255 mmol), affording 0.010 g (11%) of the title compound.

¹H NMR (DMSO-d₆) δ ppm;), 8.51 (d, 2 H), 8.32-8.47 (m, 2 H), 8.13 (d, 2 H) 7.55 (s, 1 H), 7.16 (d, 2 H), 3.87 (s, 3 H), 3.30 (s, 3 H); MS (ESI) m/z 378 (M−1).

### Example 60

### 7-(4-Methoxyphenyl)-2-(1H-pyrrol-2-yl)-3H-imidazo[4,5-b]pyridine

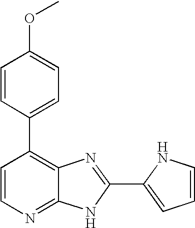

The title compound was prepared in accordance with the general method D, except that the salt was not prepared, using 4-(4-methoxyphenyl)pyridine-2,3-diamine (obtained from Example 1(c)) (50 mg, 0.232 mmol) and 2-pyrrolecarboxylic acid (26 mg, 0.255 mmol), affording 0.003 g (4.5%) of the title compound.

¹H NMR (DMSO-d₆) δ ppm; 13.12 (s, 1 H), 11.67 (s, 1 H), 8.32-8.48 (m, 2 H), 8.20 (d, 1 H), 7.43 (d, 1 H), 7.07-7.12 (m, 2 H), 6.96-7.03 (m, 2 H), 6.12-6.30 (m, 1 H), 3.85 (s, 3 H); MS (ESI) m/z 289 (M−1).

### Example 61

### 7-(4-Methoxyphenyl)-2-pyridazin-4-yl-3H-imidazo[4,5-b]pyridine

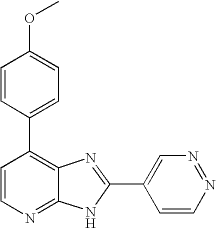

The title compound was prepared in accordance with the general method D, except that the salt was not prepared, using 4-(4-methoxyphenyl)pyridine-2,3-diamine (obtained from Example 1(c)) (50 mg, 0.232 mmol) and 4-pyridazinecarboxylic acid (29 mg, 0.255 mmol), affording 0.002 g (3%) of the title compound.

¹H NMR (DMSO-d₆) δ ppm; 10.00 (d, 1 H), 9.47 (dd, 1 H), 8.27-8.55 (m, 4 H), 7.53-7.67 (m, 1 H), 7.06-7.23 (m, 2 H), 3.87 (s, 3 H); MS (ESI) m/z 302 (M−1).

### Example 62

### 5-[7-(4-Methoxyphenyl)-3H-imidazo[4,5-b]pyridin-2-yl]pyridine-2-carbonitrile

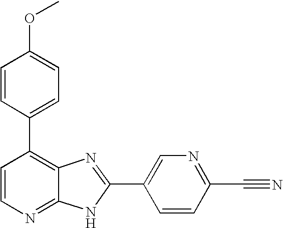

The title compound was prepared in accordance with the general method D, except that the salt was not prepared, using 4-(4-methoxyphenyl)pyridine-2,3-diamine (obtained from Example 1(c)) (50 mg, 0.232 mmol) and 2- cyano-5-carboxypyridine (34 mg, 0.255 mmol), affording 0.007 g (9%) of the title compound.

¹H NMR (DMSO-d₆) δ ppm; 9.57 (d, 1 H), 8.80 (dd, 1 H), 8.42 (d, 3 H), 8.26 (d, 1 H), 7.51-7.61 (m, 1 H), 7.01-7.23 (m, 2 H), 3.86 (s, 3 H); MS (ESI) m/z 326 (M−1).

### Example 63

### 7-(4-Methoxyphenyl)-2-(6-methylpyridin-3-yl)-3H-imidazo[4,5-b]pyridine

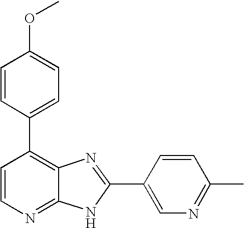

The title compound was prepared in accordance with the general method D, except that the salt was not prepared, using 4-(4-methoxyphenyl)pyridine-2,3-diamine (obtained from Example 1(c)) (50 mg, 0.232 mmol) and 6- methylpyridine-3-carboxylic acid (32 mg, 0.255 mmol), affording 0.007 g (9%) of the title compound.

¹H NMR (DMSO-d₆) δ ppm; 9.24-9.37 (m, 1 H), 8.45-8.51 (m, 1 H), 8.37-8.45 (m, 2 H), 8.31-8.35 (m, 1 H), 7.43-7.54 (m, 2 H), 7.12-7.18 (m, 2 H), 3.86 (s, 3 H), 2.57 (s, 3 H); MS (ESI) m/z 315 (M−1).

### Example 64

### 7-(4-Methoxyphenyl)-2-(1-methylcyclopropyl)-3H-imidazo[4,5-b]pyridine

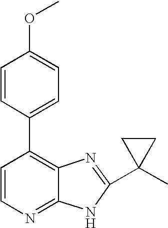

The title compound was prepared in accordance with the general method D, except that the salt was not prepared, using 4-(4-methoxyphenyl)pyridine-2,3-diamine (obtained from Example 1(c)) (50 mg, 0.232 mmol) and 1-methylcyclopropane-1-carboxylic acid (23 mg, 0.255 mmol), affording 0.007 g (11%) of the title compound.

¹H NMR (DMSO-d₆) δ ppm; 8.28-8.36 (m, 2 H), 8.19 (d, 1 H), 7.40 (d, 1 H), 7.06-7.11 (m, 2 H), 3.83 (s, 3 H), 1.58 (s, 3 H), 1.23-1.35 (m, 2 H), 0.87-0.98 (m, 2 H); MS (ESI) m/z 278 (M−1).

### Example 65

### 2-(2-Furylmethyl)-7-(4-methoxyphenyl)-3H-imidazo[4,5-b]pyridine

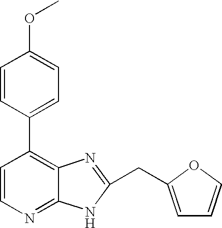

The title compound was prepared in accordance with the general method D, except that the salt was not prepared, using 4-(4-methoxyphenyl)pyridine-2,3-diamine (obtained from Example 1(c)) (50 mg, 0.232 mmol) and 2-furyl acetic acid (29 mg, 0.255 mmol), affording 0.006 g (8.5%) of the title compound.

¹H NMR (DMSO-d₆) δ ppm; 8.28-8.32 (m, 2 H), 8.26 (d, 1 H), 7.55-7.58 (m, 1 H), 7.45 (d, 1 H), 7.07-7.12 (m, 2 H), 6.41 (dd, 1 H), 6.29 (d, 1 H), 4.29 (s, 2 H), 3.83 (s, 3 H); MS (ESI) m/z 304 (M−1).

### Example 66

### 2-(Butoxymethyl)-7-(4-methoxyphenyl)-3H-imidazo[4,5-b]pyridine

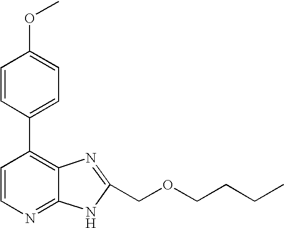

The title compound was prepared in accordance with the general method D, except that the salt was not prepared, using 4-(4-methoxyphenyl)pyridine-2,3-diamine (obtained from Example 1 (c)) (50 mg, 0.232 mmol) and butoxyacetic acid (31 mg, 0.255 mmol), affording 0.012 g (17%) of the title compound.

¹H NMR (DMSO-d₆) δ ppm; 8.22-8.36 (m, 3 H), 7.47 (d, 1 H), 7.02-7.13 (m, 3 H), 3.52 (t, 2 H), 1.47-1.61 (m, 2 H), 1.27-1.40 (m, 2 H), 0.87 (t, 3 H); MS (ESI) m/z 310 (M−1).

### Example 67

### 2-(Methoxymethyl)-7-(4-methoxyphenyl)-3H-imidazo[4,5-b]pyridine

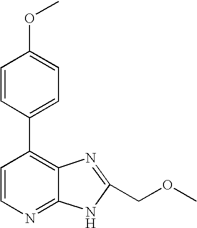

The title compound was prepared in accordance with the general method D, except that the salt was not prepared, using 4-(4-methoxyphenyl)pyridine-2,3-diamine (obtained from Example 1(c)) (50 mg, 0.232 mmol) and 1-methoxyacetic acid (23 mg, 0.255 mmol), affording 0.012 g (19%) of the title compound.

¹H NMR (DMSO-d₆) δ ppm; 8.10-8.52 (m, 3 H), 7.47 (s, 1 H), 7.11 (d, 2 H), 4.66 (s, 2 H), 3.38 (s, 3 H); MS (ESI) m/z 268 (M−1).

### Example 68

### 3-[7-(4-Methoxyphenyl)-3H-imidazo[4,5-b]pyridin-2-yl]-N-(3-methoxypropyl)benzamide hydrochloride

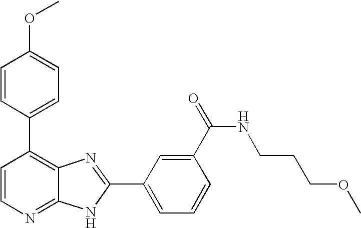

The title compound was prepared in accordance with the general method E using 3-[7-(4-methoxyphenyl)-3H-imidazo[4,5-b]pyridin-2-yl]benzoic acid (obtained from Example 68(b)) (0.100 g, 0.289 mmol), TSTU (0.105 g, 0.348 mmol), triethylamine (0.088 g, 0.87 mmol) and 2-methoxyethylamine (0.031 g, 0.413 mmol), affording 0.008 g (6%) of the title compound.

¹H NMR (DMSO-d₆) δ ppm; 8.77 (s, 1 H), 8.65 (t, 1 H), 8.39-8.50 (m, 2 H), 8.23-8.34 (m, 2 H), 7.99 (d, 1 H), 7.69 (t, 1 H), 7.59 (d, 1 H), 7.18 (d, 2 H), 3.83-3.91 (m, 3 H), 3.22-3.28 (m, 3 H), 1.64-1.89 (m, 2 H), 1.07 (s, 1 H); MS (APPI) m/z 417 (M+1)

### Example 68(a)

### Methyl 3-[7-(4-methoxyphenyl)-3H-imidazo[4,5-b]pyridin-2-yl]benzoate

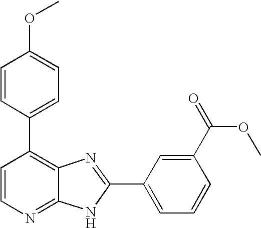

A suspension of isophtalic acid monomethyl ester (0.460 g, 2.56 mmol) and CDI (0.414 g, 2.56 mmol) in DMF (3 mL) was stirred at 0° C. for 30 mins. 4-(4-methoxyphenyl)pyridine-2,3-diamine (obtained from Example 1(c)) (0.500 g, 2.32 mmol) was added and the reaction mixture was heated to +200° C. for 10 minutes. Water was added and the product precipitated at r.t. and was filtered, washed with water and dried to afford 0.59 g (71% yield) of the title compound.

MS (APPI) m/z 360 (M+1)

### Example 68(b)

### 3-[7-(4-Methoxyphenyl)-3H-imidazo[4,5-b]pyridin-2-yl]benzoic acid

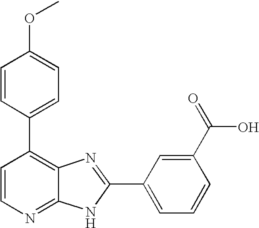

A mixture of methyl 3-[7-(4-methoxyphenyl)-3H-imidazo[4,5-b]pyridin-2-yl]benzoate (obtained from Example 68(a)) (1.0 g, 2.786 mmol) and lithium hydroxide (0.500 g, 20.8 mmol) in THF/water (9:1) was heated in microwave reactor at +60° C. for 2 h. After cooling to r.t. the mixture was made neutral using 2M HCl (aq.). The precipitate was filtered, washed with water and dried to afford the title compound in 0.84 g (87%) yield.

MS (APPI) m/z 346 (M+1)

### Example 69

### 3-{7-[4-(Aminocarbonyl)phenyl]-3H-imidazo[4,5-b]pyridin-2-yl}-N-(3-methoxypropyl)benzamide hydrochloride

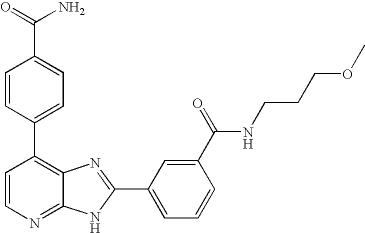

The title compound was prepared in accordance with the general method E using 3-{7-[4-(aminocarbonyl)phenyl]-3H-imidazo[4,5-b]pyridin-2-yl}benzoic acid (obtained from Example 69(c)) (0.080 g, 0.220 mmol), TSTU (0.105 g, 0.267 mmol), triethylamine (0.097 g, 0.963 mmol) and 3-methoxypropylamine (0.024 g, 0.267 mmol), affording 0.013 g (12.6%) of the title compound.

¹H NMR (DMSO-d₆) δ ppm; 8.45 (d, 1 H), 8.37-8.43 (m, 3 H), 8.04 (d, 2 H), 7.98 (d, 1 H), 7.68 (t, 1 H), 7.62 (d, 1 H), 3.22-3.28 (m, 3 H), 1.75-1.84 (m, 4 H); RT (HPLC) 6.337 min.

### Example 69(a)

### Methyl 3-(3H-imidazo[4,5-b]pyridin-2-yl)benzoate

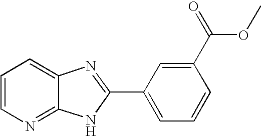

A suspension of isophtalic acid monomethyl ester (5.0 g, 28 mmol) and CDI (4.5 g, 28 mmol) in DMF (10 mL) was stirred at 0° C. for 30 min. Pyridine-2,3-diamine (2.52 g, 23 mmol) was added and the reaction mixture was heated to +200° C. for 10 minutes. Water was added and the product precipitated at r.t. and was filtered, washed with water and dried to afford 4.1 g (76% yield) of the title compound.

MS (APPI) m/z 254 (M+1).

### Example 69(b)

### Methyl 3-(7-chloro-3H-imidazo[4,5-b]pyridin-2-yl)benzoate

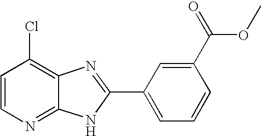

Methyl 3-(3H-imidazo[4,5-b]pyridin-2-yl)benzoate (3.0 g, 11.9 mmol, obtained from Example 69(a)) and m-CPBA (70%, 7.9 g, 35.6 mmol) in HOAc was stirred at r.t. for 18 h. The solvent was evaporated in vacuo and the residue was crystallized from EtOH. The solid was mixed with POCl₃ and heated in a microwave reactor at +100° C. for 10 minutes. After cooling to r.t., the mixture was poured into ice/water mixture and the precipitate that formed was collected, washed with water and dried, affording the title compound in 3.1 g (91%) yield.

MS (APPI) m/z 288 (M+1).

### Example 69(c)

### 3-[7-[4-(Aminocarbonyl)phenyl]-3H-imidazo[4,5-b]pyridin-2-yl}benzoic acid

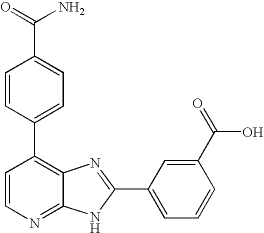

A mixture of methyl 3-{7-[4-(aminocarbonyl)phenyl]-3H-imidazo[4,5-b]pyridin-2-yl}benzoate (0.300 g, 0.806 mmol, obtained from Example 69(b)) and lithium hydroxide (0.150 g, 6.25 mmol) in THF/water (9:1) was heated in microwave reactor at +60° C. for 2 h. After cooling to r.t. the mixture was made neutral using 2M HCl (aq.). The precipitate was filtered, washed with water and dried to afford the title compound in 0.164 g (57%) yield.

MS (APPI) m/z 359-360 (M+1).

### Example 70

### 4-{2-[3-(Morpholin-4-ylcarbonyl)phenyl]-3H-imidazo[4,5-b]pyridin-7-yl}benzamide hydrochloride

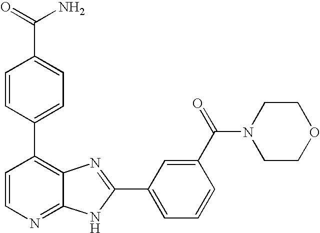

The title compound was prepared in accordance with the general method E using 3-{7-[4-(aminocarbonyl)phenyl]-3H-imidazo[4,5-b]pyridin-2-yl}benzoic acid (obtained from Example 69(c)) (0.080 g, 0.220 mmol), TSTU (0.105 g, 0.276 mmol), triethylamine (0.097 g, 0.963 mmol) and morpholine (0.024 g, 0.267 mmol), affording 0.009 g (9%) of the title compound.

¹H NMR (DMSO-d₆) δ ppm; 8.45 (d, 1 H), 8.35-8.42 (m, 3 H), 8.30-8.33 (m, 1 H), 7.54-7.74 (m, 5 H), 3.55-3.76 (m, 6 H); RT (HPLC) 7.162 min.

### Example 71

### N-(2-Methoxyethyl)-3-[7-(4-methoxyphenyl)-3H-imidazo[4,5-b]pyridin-2-yl]benzamide hydrochloride

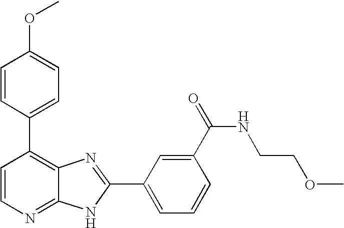

The title compound was prepared in accordance with the general method E using 3-[7-(4-methoxyphenyl)-3H-imidazo[4,5-b]pyridin-2-yl]benzoic acid (obtained from Example 68(b)) (0.100 g, 0.289 mmol), TSTU (0.105 g, 0.348 mmol), triethylamine (0.088 g, 0.87 mmol) and 2-methoxypropylamine (0.031 g, 0.348 mmol), affording 0.015 g (12%) of the title compound.

¹H NMR (DMSO-d₆) δ ppm; 8.78 (s, 1 H), 8.66-8.73 (m, 1 H), 8.39-8.49 (m, 2 H), 8.28 (d, 2 H), 8.01 (d, 1 H), 7.69 (t, 1 H), 7.59 (d, 1 H), 7.18 (d, 2 H), 3.88 (s, 3 H), 3.33-3.62 (m, 67 H), 3.30 (s, 3 H); MS (APPI) m/z 403 (M+1).

### Example 72

### 3-[7-(4-Methoxyphenyl)-3H-imidazo[4,5-b]pyridin-2-yl]-N-(2-pyrrolidin-1-ylethyl)benzamide hydrochloride

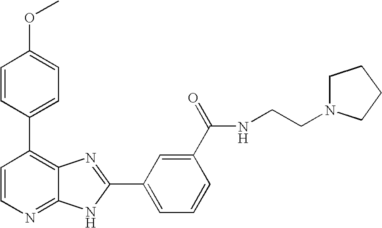

The title compound was prepared in accordance with the general method E using 3-[7-(4-methoxyphenyl)-3H-imidazo[4,5-b]pyridin-2-yl]benzoic acid (0.100 g, 0.289 mmol, obtained from Example 68(b)), TSTU (0.105 g, 0.348 mmol), triethylamine (0.088 g, 0.87 mmol) and N-(2-aminoethyl)pyrrolidine (0.040 g, 0.348 mmol), affording 0.016 g (11%) of the title compound.

¹H NMR (DMSO-d₆) δ ppm; 10.40 (s, 1 H), 8.96-9.06 (m, 1 H), 8.83 (s, 1 H), 8.37 (d, 1 H), 8.26-8.33 (m, 3 H), 8.04 (d, 1 H), 7.64 (t, 1 H), 7.48 (d, 1 H), 7.10 (d, 2 H), 3.80 (s, 3 H), 3.53-3.69 (m, 2 H), 2.92-3.07 (m, 2 H), 1.90-1.99 (m, 2 H), 1.76-1.89 (m, 2 H),

MS (APPI) m/z 442 (M+1)

### Example 73

### N-(2-Cyanoethyl)-3-[7-(4-methoxyphenyl)-3H-imidazo[4,5-b]pyridin-2-yl]benzamide hydrochloride

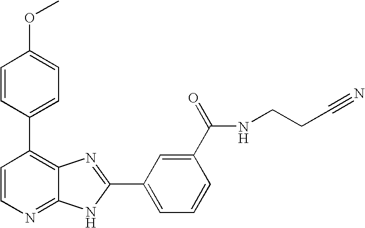

The title compound was prepared in accordance with the general method E using 3-[7-(4-methoxyphenyl)-3H-imidazo[4,5-b]pyridin-2-yl]benzoic acid (0.100 g, 0.289 mmol, obtained from Example 68(b)), TSTU (0.105 g, 0.348 mmol), triethylamine (0.088 g, 0.87 mmol) and 3-aminopropionitrile (0.024 g, 0.348 mmol), affording 0.049 g (39%) of the title compound.

¹H NMR (DMSO-d₆) δ ppm; 9.05 (t, 1 H), 8.80 (s, 1 H), 8.39-8.53 (m, 2 H), 8.25-8.35 (m, 2 H), 8.02 (d, 1 H), 7.72 (t, 1 H), 7.58 (d, 1 H), 7.18 (d, 2 H), 3.84-3.91 (m, 3 H), 3.50-3.62 (m, 2 H), 2.83 (t, 2 H); MS (APPI) m/z 398 (M+1).

### Example 74

### 7-(4-Methoxyphenyl)-2-[3-(morpholin-4-ylcarbonyl)phenyl]-3H-imidazo[4,5-b]pyridine hydrochloride

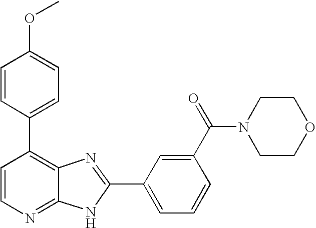

The title compound was prepared in accordance with the general method E using 3-[7-(4-methoxyphenyl)-3H-imidazo[4,5-b]pyridin-2-yl]benzoic acid (0.100 g, 0.289 mmol, obtained from Example 68(b)), TSTU (0.105 g, 0.348 mmol), triethylamine (0.088 g, 0.87 mmol) and morpholine (0.030 g, 0.348 mmol), affording 0.014 g (11%) of the title compound.

MS (APPI) m/z 415 (M+1)); RT (HPLC) 8.348 min.

### Example 75

### 7-(4-Methoxyphenyl)-2-{3-[(4-methylpiperazin-1-yl)carbonyl]phenyl}-3H-imidazo[4,5-b]pyridine hydrochloride

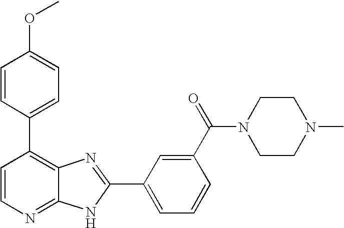

The title compound was prepared in accordance with the general method E using 3-[7-(4-methoxyphenyl)-3H-imidazo[4,5-b]pyridin-2-yl]benzoic acid (0.100 g, 0.289 mmol, obtained from Example 68(b)), TSTU (0.105 g, 0.348 mmol), triethylamine (0.088 g, 0.87 mmol) and 1-methylpiperazine (0.035 g, 0.348 mmol), affording 0.012 g (8%) of the title compound.

¹H NMR (DMSO-d₆) δ ppm; 11.23 (s, 1 H), 8.40-8.49 (m, 3 H), 8.27 (d, J=7.78 Hz, 2 H), 7.69-7.75 (m, 1 H), 7.65-7.68 (m, 1 H), 7.61 (d, J=5.27 Hz, 1 H), 7.18 (d, J=8.78 Hz, 2 H), 3.87 (s, 3 H), 3.00-3.66 (m, 58 H), 2.79 (d, J=2.26 Hz, 3 H),

MS (APPI) m/z 428 (M+1).

### Example 76

### 3-[7-(4-Methoxyphenyl)-3H-imidazo[4,5-b]pyridin-2-yl]-N-pyridin-3-ylbenzamide hydrochloride

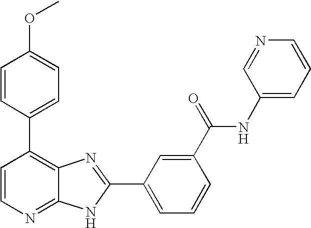

The title compound was prepared in accordance with the general method E using 3-[7-(4-methoxyphenyl)-3H-imidazo[4,5-b]pyridin-2-yl]benzoic acid (0.100 g, 0.289 mmol, obtained from Example 68(b)), TSTU (0.105 g, 0.348 mmol), triethylamine (0.088 g, 0.87 mmol) and 3-aminopyridine (0.033 g, 0.348 mmol), affording 0.019 g (13%) of the title compound.

¹H NMR (DMSO-d₆) δ ppm; 11.50 (s, 1 H), 9.42 (s, 1 H), 9.05 (s, 1 H), 8.82 (d, 2 H), 8.66 (d, 1 H), 8.57 (d, 1 H), 8.48 (d, 1 H), 8.27 (t, 3 H), 8.02 (dd, 1 H), 7.83 (t, 1 H), 7.63 9d, 1 H), 7.19 (d, 2 H), 3.80-3.98 (m, 3 H); MS (APPI) m/z 422 (M+1).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt/solvate (active ingredient) is in association with a pharmaceutically acceptable excipient, diluent or carrier. Dependent on the mode of administration, the pharmaceutical composition may comprise from 0.05 to 99% w (per cent by weight), for example from 0.10 to 50% w, of active ingredient, all percentages by weight being based on total composition.

An excipient, diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt, solvate or solvate of salt thereof, as hereinbefore defined, with a pharmaceutically acceptable excipient, diluent or carrier.

An example of a pharmaceutical composition of the invention is an injectable solution containing a compound of the invention, or a a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

### Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting of predemented states, Mild Cognitive Impairment, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment, vascular is dementia, dementia with Lewy bodies, Frontotemporal dementia and androgenetic alopecia and Type I and Type II diabetes, diabetic neuropathy and diabetes related disorders.

One embodiment of the invention relates to the prevention and/or treatment of dementia and Alzheimer's Disease.

Another embodiment of the invention relates to the prevention and/or treatment of bone-related disorders.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, including man in need of such treatment and/or prevention a therapeutically effective amount of a compound of formula I, as hereinbefore defined.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3 (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 EM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:
MOPS Morpholinepropanesulfonic acid
EDTA Ethylenediaminetetraacetic acid
BSA Bovin Serum Albumin
ATP Adenosine Triphosphate
SPA Scintillation Proximity Assay
GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

TABLE 1
Specimen results from assay.

| Example no | Kᵢ (nM) | n |
|---|---|---|
| 2 | 124 | 2 |
| 6 | 31 | 3 |
| 7 | 44 | 3 |
| 9 | 254 | 2 |

1. A compound of formula I 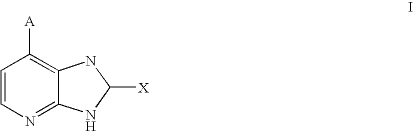
wherein
X is 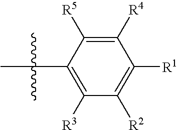
or Y;
R¹ is selected from hydrogen, halogen, CN, CO₂H, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
A is phenyl or pyridyl, optionally substituted with one or more CN, CO₂H, C₁₋₆alkyl, C₁₋₆haloalkyl, halo,C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};
Y is selected from Z, C₁₋₆alkyl, CH₂OR^{d}, and CH₂Z;
Z is heteroaryl optionally substituted with one or more CN, C₁₋₆alkyl, C₁₋₆haloalkyl, halo, C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};
R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;
R^{b} and R^{c} are independently selected from hydrogen, heteroaryl, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl or C₁₋₃haloalkyl, wherein said C₁₋₃alkyl or C₁₋₃haloalkyl is optionally further substituted with one or more C₁₋₃alkoxy;
R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, said C₁₋₃alkyl or C₁₋₃haloalkyl, optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{j} is aryl or heteroaryl, wherein said aryl or heteroaryl is optionally substituted with one or more C₁₋₃alkyl, OR^{a}, halo or CN;
R^{k} is C₁₋₆alkyl or C₁₋₆haloalkyl, wherein said C₁₋₆alkyl or C₁₋₆haloalkyl is optionally substituted with at least one CN, OR^{a}, NR^{b}R^{c}, C(O)NR^{b}R^{c} or NR^{b}C(O)R^{c};
R^{m} is C₁₋₃alkyl, optionally substituted with at least one halo, CN, OR^{a}, NR^{b}R^{c} or C(O)NR^{b}R^{c};
n is 0 to 2;
as a base or a pharmaceutically acceptable salt, solvate or solvate of a salt thereof.

2. A compound of formula I: 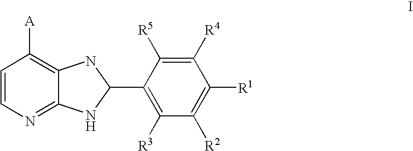
wherein
R¹ is hydrogen, halogen, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ or C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
A is phenyl or pyridyl, optionally substituted with one or more CN, C₁₋₆alkyl, C₁₋₆haloalkyl halo, OR^{k}, C(O)NR^{b}R^{c} or S(O)ₙR^{m}, said C₁₋₆alkyl or C₁₋₆haloalkyl optionally substituted by at least one OR^{a} or NR^{b}R^{c};
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl and C₁₋₆haloalkyl, wherein said C₁₋₆alkyl or C₁₋₆haloalkyl optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{d} and R^{e} are independently selected from hydrogen, C₁₋₆alkyl or C₁₋₆haloalkyl, said C₁₋₆alkyl or C₁₋₆haloalkyl optionally substituted with one or more OR^{a}; or R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{j} is aryl or heteroaryl, wherein said aryl or heteroaryl is optionally substituted with one or more C₁₋₃alkyl, OR^{a}, halo or CN;
R^{k} is C₁₋₆alkyl or C₁₋₆haloalkyl, optionally substituted with at least one CN, OR^{a}, NR^{b}R^{c}, or C(O)NR^{b}R^{c};
R^{m} is C₁₋₃alkyl, optionally substituted with at least one halo, CN, OR^{a}, NR^{b}R^{c} or C(O)NR^{b}R^{c};
n is 0 to 2;
as a base or a pharmaceutically acceptable salt, solvate or solvate of a salt thereof.

3. A compound according to claim 1, wherein
R¹ is hydrogen, halogen, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ or C(O)R^{j};
R² and R⁴ are independently selected from hydrogen, halo, CN, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
A is phenyl or pyridyl, optionally substituted with one or more CN, C₁₋₆alkyl, C₁₋₆haloalkyl, halo, OR^{k}, C(O)NR^{b}R^{c} or S(O)ₙR^{m}, wherein said C₁₋₆alkyl or C₁₋₆haloalkyl is optionally substituted by at least one OR^{a} or NR^{b}R^{c};
R^{a} is hydrogen, C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl and C₁₋₆haloalkyl, wherein said C₁₋₆alkyl or C₁₋₆haloalkyl is optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, said C₁₋₃alkyl or C₁₋₃haloalkyl optionally substituted with one or more C₁₋₃alkoxy;
Rⁱ is C₁₋₃alkyl or C₁₋₃haloalkyl, wherein said C₁₋₃alkyl or C₁₋₃haloalkyl is optionally substituted with one or more OR^{a};
R^{j} is aryl or heteroaryl, wherein said aryl or heteroaryl is optionally substituted with one or more C₁₋₃alkyl, OR^{a}, halo or CN;
R^{k} is C₁₋₆alkyl or C₁₋₆haloalkyl, optionally substituted with at least one CN, OR^{a}, NR^{b}R^{c} or C(O)NR^{b}R^{c};
R^{m} is C₁₋₃alkyl, optionally substituted with at least one halo, CN, OR^{a}, NR^{b}R^{c} or C(O)NR^{b}R^{c};
n is 0 to 2;
as a base or a pharmaceutically acceptable salt, solvate or solvate of a salt thereof.

4. A compound according to claim 1, wherein
R¹ is hydrogen, SO₂NR^{b}R^{c}, C(O)NR^{b}R^{c}, CH₂NR^{b}R^{c}, CH₂OR^{h}, or SO₂Rⁱ;
R² and R⁴ are independently selected from hydrogen, halo, CN, NO₂, C₁₋₃alkyl, C₁₋₃haloalkyl, OR^{a}, C(O)NR^{b}R^{c}, CH₂NR^{b}R^{c}, CH₂OR^{h} and SO₂Rⁱ;
R³ and R⁵ are hydrogen;
A is phenyl or pyridyl, optionally substituted with one or more CN, C₁₋₆alkyl, halo, OR^{k} or C(O)NR^{b}R^{c}, said C₁₋₆alkyl optionally substituted by at least one OR^{a} or NR^{b}R^{c};
R^{a} is C₁₋₃alkyl or C₁₋₃haloalkyl, said C₁₋₃alkyl or C₁₋₃haloalkyl optionally substituted with one or more C₁₋₃alkoxy;
R^{b} and R^{c} are independently selected from hydrogen, C₁₋₆alkyl or C₁₋₆haloalkyl, wherein said C₁₋₆alkyl or C₁₋₆haloalkyl is optionally substituted with one or more OR^{a} or NR^{d}R^{e} or R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{d} and R^{e} form, together with the atom to which they are attached, a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl;
Rⁱ is C₁₋₃alkyl or C₁₋₃haloalkyl;
R^{k} is C₁₋₆alkyl or C₁₋₆haloalkyl, optionally substituted with at least one CN, OR^{a}, NR^{b}R^{c}, or C(O)NR^{b}R^{c};
as a base or a pharmaceutically acceptable salt, solvate or solvate of a salt thereof.

5. A compound according to claim 1, wherein
R¹ is SO₂NR^{b}R^{c}, C(O)NR^{b}R^{c} or CH₂NR^{b}R^{c};
R², R³, R⁴ and R⁵ are hydrogen;
A is phenyl or pyridyl, optionally substituted with one or more CN, C₁₋₆alkyl, halo, OR^{k} or C(O)NR^{b}R^{c}, wherein said C₁₋₆alkyl is optionally substituted by at least one NR^{b}R^{c};
R^{b} and R^{c} are independently selected from hydrogen or C₁₋₆alkyl, wherein said C₁₋₆alkyl is optionally substituted with one or more NR^{d}R^{e} or R^{b} and R^{c} may, together with the atom to which they are attached, form a 6-membered heterocyclic ring containing one or more heteroatoms selected from N or O, wherein said heterocyclic ring is optionally substituted with one or more C₁₋₃alkyl;
R^{d} and R^{e} form, together with the atom to which they are attached, a 6-membered heterocyclic ring containing one or more heteroatoms selected from N, O or S;
R^{k} is C₁₋₆alkyl or C₁₋₆haloalkyl;
as a base or a pharmaceutically acceptable salt, solvate or solvate of a salt thereof.

6. A compound according to claim 1, wherein
R¹ is selected from hydrogen, halogen, CN, CO₂H, NO₂, OR^{a}, SO₂NR^{b}R^{c}, C(O)NR^{b}R^{c}, CH₂NR^{b}R^{c}, CH₂OR^{h}, SO₂Rⁱ and C(O)R^{j};
R² and R⁴ are independently selected from hydrogen, halo, CN, NO₂, OR^{a}, SO₂NR^{b}R^{c}, C(O)NR^{b}R^{c}, CH₂NR^{b}R^{c}, CH₂OR^{h}, SO₂Rⁱ and C(O)R^{j};
R³ and R⁵ are independently selected from hydrogen, C₁₋₃alkyl and C₁₋₃haloalkyl;
A is phenyl or pyridyl, optionally substituted with one or more CN, CO₂H, C₁₋₆alkyl, C₁₋₆haloalkyl, halo,C(O)R^{a}, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};
Y is selected from Z, C₁₋₆alkyl, CH₂OR^{d}, and CH₂Z;
Z is heteroaryl optionally substituted with one or more CN, C₁₋₆alkyl C₁₋₆haloalkyl, halo, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};
R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;
R^{b} and R^{c} are independently selected from hydrogen, heteroaryl, C₁₋₆alkyl and C₁₋₆haloalkyl, wherein said C₁₋₆alkyl or C₁₋₆haloalkyl optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;
R^{d} and R^{e} are independently selected from hydrogen, C₁₋₆alkyl or C₁₋₆haloalkyl, wherein said C₁₋₆alkyl or C₁₋₆haloalkyl is optionally substituted with one or more OR^{a}; or R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, said C₁₋₃alkyl or C₁₋₃haloalkyl, optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{j} is aryl or heteroaryl, wherein said aryl or heteroaryl is optionally substituted with one or more C₁₋₃alkyl, OR^{a}, halo or CN;
R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};
R^{m} is C₁₋₃alkyl, optionally substituted with at least one halo, CN, OR^{a}, NR^{b}R^{c} or C(O)NR^{b}R^{c};
n is 0 to 2;
as a base or a pharmaceutically acceptable salt, solvate or solvate of a salt thereof.

7. A compound according to claim 1, wherein
R¹ is selected from hydrogen, halogen, CO₂H, NO₂, OR^{a}, SO₂NR^{b}R^{c}, C(O)NR^{b}R^{c}, CH₂NR^{b}R^{c}, CH₂OR^{h}, and SO₂Rⁱ;
R² and R⁴ are independently selected from hydrogen, halo, OR^{a}, SO₂NR^{b}R^{c}, C(O)NR^{b}R^{c}, CH₂NR^{b}R^{c}, CH₂OR^{h}, and SO₂Rⁱ;
R³ and R⁵ are independently selected from hydrogen, C₁₋₃alkyl and C₁₋₃haloalkyl;
A is phenyl or pyridyl, optionally substituted with one or more CN, CO₂H, C₁₋₆alkyl, C₁₋₆haloalkyl, halo, C(O)R^{a}, OR^{k}, or C(O)NR^{b}R^{c}, wherein said C₁₋₆alkyl or C₁₋₆haloalkyl is optionally substituted by at least one CN, OR^{a} or NR^{b}R^{c};
Y is selected from Z, C₁₋₆alkyl, CH₂OR^{d}, and CH₂Z
Z is heteroaryl optionally substituted with one or more CN, C₁₋₆alkyl C₁₋₆haloalkyl, halo, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c};
R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;
R^{b} and R^{c} are independently selected from hydrogen, heteroaryl, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl wherein said C₁₋₃alkyl is optionally further substituted with one or more C₁₋₃alkoxy;
R^{d} and R^{e} are independently selected from hydrogen, C₁₋₆alkyl or C₁₋₆haloalkyl, said C₁₋₆alkyl or C₁₋₆haloalkyl optionally substituted with one or more OR^{a}; or R^{d} and R^{e} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, said C₁₋₃alkyl or C₁₋₃haloalkyl, optionally substituted with one or more C₁₋₃alkoxy;
Rⁱ is C₁₋₃alkyl or C₁₋₃haloalkyl, said C₁₋₃alkyl or C₁₋₃haloalkyl optionally substituted with one or more OR^{a};
R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};
R^{m} is C₁₋₃alkyl, optionally substituted with at least one halo, CN, OR^{a}, NR^{b}R^{c} or C(O)NR^{b}R^{c};
as a base or a pharmaceutically acceptable salt, solvate or solvate of a salt thereof.

8. A compound according to claim 1, wherein
R¹ is selected from hydrogen, CO₂H, SO₂NR^{b}R^{c}, C(O)NR^{b}R^{c}, CH₂NR^{b}R^{c}, and SO₂Rⁱ;
R² and R⁴ are independently selected from hydrogen, C(O)NR^{b}R^{c}, CH₂NR^{b}R^{c}, and SO₂Rⁱ;
R³ and R⁵ are hydrogen;
A is phenyl or pyridyl, optionally substituted with one or more CN, CO₂H, C₁₋₆alkyl, halo, C(O)R^{a}, OR^{k}, C(O)NR^{b}R^{c} or S(O)ₙR^{m}, wherein said C₁₋₆alkyl is optionally substituted by at least one CN, OR^{a} or NR^{b}R^{c};
Y is selected from Z, C₁₋₆alkyl, CH₂OR^{d}, and CH₂Z;
Z is heteroaryl optionally substituted with one or more CN, C₁₋₆alkyl or C(O)NR^{b}R^{c};
R^{a} is selected from hydrogen and C₁₋₃alkyl, wherein said C₁₋₃alkyl is optionally substituted with one or more C₁₋₃alkoxy;
R^{b} and R^{c} are independently selected from hydrogen, heteroaryl and C₁₋₆alkyl, wherein said C₁₋₆alkyl is optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl, wherein said C₁₋₃alkyl is optionally further substituted with one or more C₁₋₃alkoxy;
R^{d} and R^{e} are, C₁₋₆alkyl; or R^{d} and R^{e} may, together with the atom to which they are attached, form a 4-, 5-, 6- or 7-membered heterocyclic ring containing one or more heteroatoms selected from N or O;
Rⁱ is C₁₋₃alkyl;
R^{k} is C₁₋₆alkyl or C₁₋₆haloalkyl, wherein said C₁₋₆alkyl or C₁₋₆haloalkyl is optionally substituted with at least one CN, OR^{a} or NR^{b}C(O)R^{c};
as a base or a pharmaceutically acceptable salt, solvate or solvate of a salt thereof.

9-45. (canceled)

46. A compound according to claim 1, wherein R³ and R⁵ are hydrogen.

47. A compound according to claim 46, wherein A is pyridyl.

48. A compound according to claim 46, wherein A is phenyl, optionally substituted with one or more CN, CO₂H, C₁₋₆alkyl, C₁₋₆haloalkyl, halo, C(O)R¹, OR^{k}, C(O)NR^{b}R^{c} or S(O)ₙR^{m}, wherein said C₁₋₆alkyl or C₁₋₆haloalkyl is optionally substituted by at least one CN, OR^{a} or NR^{b}R^{c}.

49. A compound according to claim 48, wherein A is substituted with one or more CN, CO₂H, C₁₋₆alkyl, halo, C(O)R^{a}, OR^{k} or C(O)NR^{b}R^{c}, wherein said C₁₋₆alkyl is optionally substituted by at least one CN, OR^{a} or NR^{b}R^{c}.

50. A compound according to claim 49, wherein A is substituted with OR^{k}, C₁₋₆alkyl, halo or C(O)NR^{b}R^{c}.

51. A compound according to claim 50, wherein A is substituted with OR^{k} and R^{k} is C₁₋₆alkyl.

52. A compound according to claim 51, wherein R^{k} is methyl.

53. A compound according to any one of claims 49 to 52, wherein
R¹ and R² are hydrogen;
R⁴ is C(O)NR^{b}R^{c};
R^{b} and R^{c} are independently selected from hydrogen, heteroaryl and C₁₋₆alkyl, wherein said C₁₋₆alkyl is optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N or O, wherein said heterocyclic ring is optionally substituted with one or more halo, OR^{a}, NR^{d}R^{c}, C₁₋₃alkyl, wherein said C₁₋₃alkyl is optionally further substituted with one or more C₁₋₃alkoxy;
R^{a} is C₁₋₃alkyl, wherein said C₁₋₃alkyl is optionally substituted with one or more C₁₋₃alkoxy; and
R^{d} and R^{e} may, together with the atom to which they are attached, form a 5-membered heterocyclic ring containing one or more heteroatoms selected from N.

54. A compound according to any one of claims 49 to 52 wherein,
R¹ and R⁴ are hydrogen;
R² is SO₂Rⁱ; and
Rⁱ is C₁₋₃alkyl or C₁₋₃haloalkyl.

55. A compound according to claim 54, wherein Rⁱ is methyl.

56. A compound according to claim 46, wherein
R² and R⁴ are hydrogen;
A is substituted with one or more halo, OR^{k} or C(O)NR^{b}R^{c} and wherein R^{k} is C₁₋₆ alkyl; and
R^{b} and R^{c} together with the atom to which they are attached, form a 4-, 5- or 6-membered heterocyclic ring containing one or more heteroatoms selected from N or O, wherein said heterocyclic ring is optionally substituted with one or more halo, C₁₋₃alkyl or C₁₋₃haloalkyl, said C₁₋₃alkyl or C₁₋₃haloalkyl optionally further substituted with one or more C₁₋₃alkoxy.

57. A compound according to claim 56, wherein A is substituted with OR^{k} or C(O)NR^{b}R^{c}.

58. A compound according to claim 57, wherein R^{k} is C₁₋₆alkyl.

59. A compound according to claim 58, wherein R^{k} is methyl.

60. A compound according to claim 57, wherein
R^{b} and R^{c} are independently selected from hydrogen, C₁₋₆alkyl and C₁₋₆haloalkyl, wherein said C₁₋₆alkyl or C₁₋₆haloalkyl is optionally substituted with one or more CN, OR^{a} or NR^{d}R^{e}; or R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, OR^{a}, NR^{d}R^{e}, C₁₋₃alkyl or C₁₋₃haloalkyl, wherein said C₁₋₃alkyl or C₁₋₃haloalkyl is optionally further substituted with one or more C₁₋₃alkoxy.

61. A compound according to claim 60, wherein R^{b} and R^{c} together with the atom to which they are attached, form a 5-, 6- or 7-membered heterocyclic ring containing one or more heteroatoms selected from N or O, wherein said heterocyclic ring is optionally substituted with one or more halo or C₁₋₃alkyl, wherein said C₁₋₃alkyl is optionally further substituted with one or more C₁₋₃alkoxy.

62. A compound according to any one of claims 56 to 61, wherein R¹ is selected from halogen, CO₂H, C(O)NR^{b}R^{c} and CH₂NR^{b}R^{c}.

63. A compound according to claim 62, wherein
R¹ is C(O)NR^{b}R^{c} or CH₂NR^{b}R^{c}; and
R^{b} and R^{c} together with the atom to which they are attached, form a 5-, 6- or 7-membered heterocyclic ring containing one or more heteroatoms selected from N or O, wherein said heterocyclic ring is optionally substituted with one or more halo or C₁₋₃alkyl, wherein said C₁₋₃alkyl is optionally further substituted with one or more C₁₋₃alkoxy.

64. A compound in accord with claim 1, selected from:
7-(4-Methoxyphenyl)-2-{4-[(4-methylpiperazin-1-yl)sulfonyl]phenyl}-3H-imidazo[4,5-b]pyridine hydrochloride;
7-(3-Methoxyphenyl)-2-{4-[(4-methylpiperazin-1-yl)sulfonyl]phenyl}-3H-imidazo[4,5-b]pyridine hydrochloride;
7-(4-Methoxyphenyl)-2-{4-[(4-methylpiperazin-1-yl)carbonyl]phenyl}-3H-imidazo[4,5-b]pyridine hydrochloride;
7-(3-methoxyphenyl)-2-{4-[(4-methylpiperazin-1-yl)carbonyl]phenyl}-3H-imidazo[4,5-b]pyridine hydrochloride;
7-(4-Chlorophenyl)-2-{4-[(4-methylpiperazin-1-yl)carbonyl]phenyl}-3H-imidazo[4,5-b]pyridine hydrochloride;
7-(4-Methoxyphenyl)-2-[4-(piperidin-1-ylmethyl)phenyl]-3H-imidazo[4,5-b]pyridine hydrochloride;
4-[7-(4-Methoxyphenyl)-3H-imidazo[4,5-b]pyridin-2-yl]-N-(2-morpholin-4-ylethyl)benzamide hydrochloride;
2-{4-[(4-Methylpiperazin-1-yl)carbonyl]phenyl}-7-[4-(trifluoromethoxy)phenyl]-3H-imidazo[4,5-b]pyridine hydrochloride;
2-{4-[(4-Methylpiperazin-1-yl)carbonyl]phenyl}-7-pyridin-3-yl-3H-imidazo[4,5-b]pyridine hydrochloride;
7-(2,4-Dimethoxyphenyl)-2-[4-(morpholin-4-ylcarbonyl)phenyl]-3H-imidazo[4,5-b]pyridine hydrochloride;
4-(2-{4-[(4-Methylpiperazin-1-yl)carbonyl]phenyl}-3H-imidazo[4,5-b]pyridin-7-yl)benzonitrile hydrochloride;
7-(4-Methoxyphenyl)-2-[4-(morpholin-4-ylmethyl)phenyl]-3H-imidazo[4,5-b]pyridine hydrochloride;
2-{4-[4-Methylpiperazin-1-yl)carbonyl]phenyl}-7-[3-(morpholin-4-ylmethyl)phenyl]-1H-imidazo[4,5-b]pyridine;
N-(2-Cyanoethyl)-3-{2-[(4-methylpiperazin-1-yl)carbonyl]-3H-imidazo[4,5-b]pyridine-7-yl}benzamide;
7-{3-[2-(2-Methoxyethoxy)ethoxy]phenyl}-2-{4-[(4-methylpiperazin-1-yl)carbonyl]phenyl}-3H-imidazo[4,5-b]pyridine;
3-[3-(2-{4-[(4-Methylpiperazin-1-yl)carbonyl]phenyl}-3H-imidazo[4,5-b]pyridin-7-yl)propan-1-ol;
7-[3-(3-Methoxypropoxy)phenyl]-2-{4-[(4-methylpiperazin-1-yl)carbonyl]phenyl}-3H-imidazo[4,5-b]pyridine;
N-{3-[3-(2-{4-[4-Methylpiperazin-1-yl)carbonyl]phenyl}-3H-imidazo[4,5-b]pyridine-7-yl)phenoxy]propyl}acetamide;
4-[3-(2-{4-[(4-Methylpiperazin-1-yl)carbonyl]phenyl}-3H-imidazo[4,5-b]pyridine-7-yl)phenoxy]butanenitrile;
3-[3-(2-{4-[(4-Methylpiperazin-1-yl)carbonyl]phenyl}-3H-imidazo[4,5-b]pyridine-7-yl)phenoxy]propan-1-ol;
3-[3-(2-{4-[(4-Methylpiperazin-1-yl)carbonyl]phenyl}-3H-imidazo[4,5-b]pyridine-7-yl)phenoxy]acetonitrile;
7-(4-Methoxyphenyl)-2-{5-[(4-methylpiperzin-1-yl)carbonyl]pyridine-2-yl}-3H-imidazo[4,5-b]pyridine;
2-{4-[(3,3-Difluoropyrrolidin-1-yl)carbonyl]phenyl}-7-(4-methoxyphenyl)3H-imidazo[4,5-b]pyridine;
7-(4-Methoxyphenyl)-2-(4-{[(3R)-3-methylmorpholin-4-yl]carbonyl}phenyl)-3H-imidazo[4,5-b]pyridine;
2-{4-[(4-Ethylpiperazin-1-yl)carbonyl]phenyl}-7-(4-methoxyphenyl)-3H-imidazo[4,5-b]pyridine;
4-[7-(4-Methoxyphenyl)-3H-imidazo[4,5-b]pyridin-2-yl]-N-(2-piperidin-1-ylethyl)benzamide;
7-(4-Methoxyphenyl)-2-{4-[(4-methyl-1,4-diazepan-1-yl)carbonyl]phenyl}-3H-imidazo[4,5-b]pyridine;
1-{4-[7-(4-Methoxyphenyl)-3H-imidazo[4,5-b]pyridin-2-yl]benzoyl}-N,N-dimethylpyrrolidin-3-amine;
2-(4-{[4-(2-Methoxyethyl)piperazin-1-yl]carbonyl}phenyl)-7-(4-methoxyphenyl)-3H-imidazo[4,5-b]pyridine;
2-{4-[(4-Isopropylpiperazin-1-yl)carbonyl]phenyl}-7-(4-methoxyphenyl)-3H-imidazo[4,5-b]pyridine;
2-(4-{[(3S)-3-Fluoropyrrolidin-1-yl]carbonyl}phenyl)-7-(4-methoxyphenyl)-3H-imidazo[4,5-b]pyridine;
1-{4-[7-(4-Methoxyphenyl)-3H-imidazo[4,5-b]pyridin-2-yl]benzoyl}pyrrolidin-3-ol hydrochloride;
7-(3-Fluoro-4-Methoxyphenyl)-2-{4-[(4-methylpiperazin-1-yl)carbonyl]phenyl}-3H-imidazo[4,5-b]pyridine hydrochloride;
7-(4-Isopropoxyphenyl)-2-{4-[(4-methylpiperazin-1-yl)carbonyl]phenyl}-3H-imidazo[4,5-b]pyridine hydrochloride;
7-(4-Ethoxyphenyl)-2-{4-[(4-methylpiperazin-1-yl)carbonyl]phenyl}-3H-imidazo[4,5-b]pyridine hydrochloride;
7-(4-Methoxy-2-methylphenyl)-2-{4-[(4-methylpiperazin-1-yl)carbonyl]phenyl}-3H-imidazo[4,5-b]pyridine hydrochloride;
7-(4-Methoxyphenyl)-2-{2-[(4-methylpiperazin-1-yl)carbonyl]pyridin-4-yl}-3H-imidazo[4,5-b]pyridine;
7-(4-Methoxyphenyl)-2-{5-[(4-methylpiperazin-1-yl)carbonyl]pyridin-3-yl}-3H-imidazo[4,5-b]pyridine;
7-(2-Methoxyphenyl)-2-[4-(morpholin-4-ylcarbonyl)phenyl]-3H-imidazo[4,5-b]pyridine hydrochloride;
N-(3-Methoxypropyl)-4-(7-pyridin-4-yl-3H-imidazo[4,5-b]pyridin-2-yl)benzamide hydrochloride;
2-{4-[(4-Methylpiperazin-1-yl)carbonyl]phenyl}-7-pyridin-4-yl-3H-imidazo[4,5-b]pyridine hydrochloride;
2-{4-[(4-Methylpiperazin-1-yl)methyl]phenyl}-7-pyridin-4-yl-3H-imidazo[4,5-b]pyrindine hydrochloride;
4-(2-{4-[(4-Methylpiperazin-1-yl)methyl]phenyl}-3H-imidazo[4,5-b]pyridin-7-yl)benzamide hydrochloride;
7-(4-Methoxyphenyl)-2-{4-[(4-methylpiperazin-1-yl)methyl]phenyl}-3H-imidazo[4,5-b]pyridine hydrochloride;
7-(4-Ethoxyphenyl)-2-{4-[(4-methylpiperazin-1-yl)methyl]phenyl}-3H-imidazo[4,5-b]pyridine hydrochloride;
(4-{2-[4-(Morpholin-4-ylmethyl)phenyl]-3H-imidazo[4,5-b]pyridin-7-yl}phenyl)methanol hydrochloride;
N-Methyl-4-{2-[4-(morpholin-4-ylmethyl)phenyl]-3H-imidazo[4,5-b]pyridin-7-yl}benzamide hydrochloride;
2-[4-(Morpholin-4-ylmethyl)phenyl]-7-[4-(pyrrolidin-1-ylcarbonyl)phenyl]-3H-imidazo[4,5-b]pyridine hydrochloride;
4-{2-[4-(Morpholin-4-ylmethyl)phenyl]-3H-imidazo[4,5-b]pyridin-7-yl}benzamide hydrochloride;
(4-{2-[4-(Morpholin-4-ylmethyl)phenyl]-3H-imidazo[4,5-b]pyridin-7-yl}phenyl)acetonitrile hydrochloride;
4-{2-[4-(Morpholin-4-ylmethyl)phenyl]-3H-imidazo[4,5-b]pyridin-7-yl}benzoic acid hydrochloride;
4,4′-(3H-Imidazo[4,5-b]pyridine-2,7-diyl)dibenzoic acid;
7-[4-(Azetidin-1-ylcarbonyl)phenyl]-2-[4-(morpholin-4-ylmethyl)phenyl]-3H-imidazo[4,5-b]pyridine hydrochloride;
1-(4-{2-[4-(Morpholin-4-ylmethyl)phenyl]-3H-imidazo[4,5-b]pyridin-7-yl}phenyl)ethanone hydrochloride;
7-(4-Methoxyphenyl)-2-[3-(morpholin-4-ylmethyl)phenyl]-3H-imidazo[4,5-b]pyridine hydrochloride;
7-(4-Methoxyphenyl)-2-{3-[(4-methylpiperazin-1-yl)methyl]phenyl}-3H-imidazo[4,5-b]pyridine hydrochloride;
7-(4-Methoxyphenyl)-2-phenyl-3H-imidazo[4,5-b]pyridine;
7-(4-Methoxyphenyl)-2-[3-(methylsulfonyl)phenyl]-3H-imidazo[4,5-b]pyridine;
7-(4-Methoxyphenyl)-2-[4-(methylsulfonyl)phenyl]-3H-imidazo[4,5-b]pyridine;
7-(4-Methoxyphenyl)-2-(1H-pyrrol-2-yl)-3H-imidazo[4,5-b]pyridine;
7-(4-Methoxyphenyl)-2-pyridazin-4-yl-3H-imidazo[4,5-b]pyridine;
5-[7-(4-Methoxyphenyl)-3H-imidazo[4,5-b]pyridin-2-yl]pyridine-2-carbonitrile;
7-(4-Methoxyphenyl)-2-(6-methylpyridin-3-yl)-3H-imidazo[4,5-b]pyridine;
7-(4-Methoxyphenyl)-2-(1-methylcyclopropyl)-3H-imidazo[4,5-b]pyridine;
2-(2-Furylmethyl)-7-(4-methoxyphenyl)-3H-imidazo[4,5-b]pyridine;
2-(Butoxymethyl)-7-(4-methoxyphenyl)-3H-imidazo[4,5-b]pyridine;
2-(Methoxymethyl)-7-(4-methoxyphenyl)-3H-imidazo[4,5-b]pyridine;
3-[7-(4-Methoxyphenyl)-3H-imidazo[4,5-b]pyridin-2-yl]-N-(3-methoxypropyl)benzamide hydrochloride;
3-{7-[4-(Aminocarbonyl)phenyl]-3H-imidazo[4,5-b]pyridin-2-yl}-N-(3-methoxypropyl)benzamide hydrochloride;
4-{2-[3-(Morpholin-4-ylcarbonyl)phenyl]-3H-imidazo[4,5-b]pyridin-7-yl}benzamide hydrochloride;
N-(2-Methoxyethyl)-3-[7-(4-methoxyphenyl)-3H-imidazo[4,5-b]pyridin-2-yl]benzamide hydrochloride;
3-[7-(4-Methoxyphenyl)-3H-imidazo[4,5-b]pyridin-2-yl]-N-(2-pyrrolidin-1-ylethyl)benzamide hydrochloride;
N-(2-Cyanoethyl)-3-[7-(4-methoxyphenyl)-3H-imidazo[4,5-b]pyridin-2-yl]benzamide hydrochloride;
7-(4-Methoxyphenyl)-2-[3-(morpholin-4-ylcarbonyl)phenyl]-3H-imidazo[4,5-b]pyridine hydrochloride;
7-(4-Methoxyphenyl)-2-{3-[(4-methylpiperazin-1-yl)carbonyl]phenyl}-3H-imidazo[4,5-b]pyridine hydrochloride; or
3-[7-(4-Methoxyphenyl)-3H-imidazo[4,5-b]pyridin-2-yl]-N-pyridin-3-ylbenzamide hydrochloride;
as a base or a pharmaceutically acceptable salt, solvate or solvate of a salt thereof.

65. A pharmaceutical formulation comprising as active ingredient a therapeutically effective amount of a compound according to claim 1 in association with pharmaceutically acceptable excipients, carriers or diluents.

66. A method of prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, comprising administering to a mammal, including man in need of such prevention and/or treatment, a therapeutically effective amount of a compound of formula I as defined in claim 1.

67. A method according to claim 66, wherein the disease is Alzheimer's Disease.

68. A method of prevention and/or treatment of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, bone-related disorders or hair loss, comprising administering to a mammal, including man in need of such prevention and/or treatment, a therapeutically effective amount of a compound of formula I as defined in claim 1.

69. A method of prevention and/or treatment of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders, comprising administering to a mammal, including man in need of such prevention and/or treatment, a therapeutically effective amount of a compound of formula I as defined in claim 1.

70. A compound selected from:
2-(Benzyloxy)-4-(4-methoxyphenyl)-3-nitropyridine;
4-(4-Methoxyphenyl)-3-nitropyridin-2-amine;
4-(4-Methoxyphenyl)pyridine-2,3-diamine;
2-(Benzyloxy)-4-(3-methoxyphenyl)-3-nitropyridine;
4-(3-Methoxyphenyl)pyridine-2,3-diamine;
Methyl 4-[7-(4-methoxyphenyl)-3H-imidazo[4,5-b]pyridin-2-yl]benzoate;
Methyl 4-[7-(3-methoxyphenyl)-3H-imidazo[4,5-b]pyridin-2-yl]benzoate;
7-Chloro-2-{4-[(4-methylpiperazin-1-yl)carbonyl]phenyl}-3H-imidazo[4,5-b]pyridine;
7-Chloro-2-[4-(piperidin-1-ylcarbonyl)phenyl]-3H-imidazo[4,5-b]pyridine;
7-(4-Methoxyphenyl)-2-[4-(piperidin-1-ylcarbonyl)phenyl]-3H-imidazo[4,5-b]pyridine;
4-(7-Chloro-3H-imidazo[4,5-b]pyridin-2-yl)-N-(2-morpholin-4-ylethyl)benzamide;
Methyl 4-[7-(4-cyanophenyl)-3H-imidazo[4,5-b]pyridin-2-yl]benzoate;
7-(4-Methoxyphenyl)-2-[4-(morpholin-4-ylcarbonyl)phenyl]-3H-imidazo[4,5-b]pyridine;
4-[3-(Morpholin-4-ylcarbonyl)phenyl]-3-nitropyridin-2-amine;
4-[3-(Morpholin-4-ylcarbonyl)phenyl]pyridine-2,3-diamine;
4-[3-(Morpholin-4-ylmethyl)phenyl]pyridine-2,3-diamine;
Methyl 4-{7-[3-(morpholin-4-ylmethyl)phenyl]-1H-imidazo[4,5-b]pyridine-2-yl}benzoate;
4-{7-[3-(Morpholin-4-ylmethyl)phenyl]-1H-imidazo[4,5-b]pyridine-2-yl}benzoic acid;
Methyl 4-(7-iodo-3H-imidazo[4,5-b]pyridin-2-yl)benzoate;
Methyl 4-(7-iodo-3-{[2-(trimethylsilyl)ethoxy]methyl}-3H-imidazo[4,5-b]pyridine-2-yl)benzoate;
Methyl 4-(7-(3-{[(2-cyanoethyl)amino]carbonyl}phenyl)-3-{[2-(trimethylsilyl)ethoxy]methyl}-3H-imidazo[4,5-b]pyridin-2-yl)benzoate;
4-(7-(3-{[(2-Cyanoethyl)amino]carbonyl}phenyl)-3-{[2-(trimethylsilyl)ethoxy]methyl}-3H-imidazo[4,5-b]pyridin-2-yl)benzoic acid;
N-(2-Cyanoethyl)-3-(2-[(4-methylpiperazin-1-yl)carbonyl]-3-{[2-(trimethylsilyl)ethoxy]methyl}-3H-imidazo[4,5-b]pyridin-7-yl)benzamine;
Methyl 4-(7-[3-(benzyloxy)phenyl]-3H-imidazo[4,5-b]pyridine-2-yl)benzoate;
Methyl 4-(7-[3-(benzyloxy)phenyl]-3-{[2-(trimethylsilyl)ethoxy]methyl}-3H-imidazo[4,5-b]pyridine-2-yl)benzoate;
4-(7-[3-(Benzyloxy)phenyl]-3-{[2-(trimethylsilyl)ethoxy]methyl}-3H-imdazo[4,5-b]pyridine-2-yl)benzoic acid;
7-[3-(Benzyloxy)phenyl]-2-{4-[(methylpiperazin-1-yl)carbonyl]phenyl}-3-{[2-(trimethylsilyl)ethoxy]methyl}3H-imidazo[4,5-b]pyridine;
3-(2-{4-[(4-Methylpiperazin-1-yl)carbonyl]phenyl}-3-{[2-(trimethylsilyl)ethoxy]methoxy}-3H-imidazo[4,5-b]pyridin-7-yl)phenol;
Methyl 4-(7-[3-(3-hydroxypropyl)phenyl]-3-{[2-(trimethylsilyl)ethoxy]methyl}-3H-imidazo[4,5-b]pyridine-2-yl)benzoate;
4-(7-[3-(3-Hydroxypropyl)phenyl]-3-{[2-(trimethylsilyl)ethoxy]methyl}-3H-imidazo[4,5-b]pyridine-2-yl)benzoic acid;
3-[3-(2-{4-Methylpiperazin-1-yl)carbonyl]phenyl}-3-{[2-(trimethylsilyl)ethoxy]methoxy}-3H-imidazo[4,5-b]pyridin-7-yl)phenyl]propan-1-ol;
7-[3-(3-Methoxypropoxy)phenyl]-2-{4-[(4-methylpiperazin-1-yl)carbonyl]phenyl}-3H-imidazo[4,5-b]pyridine;
N-{3-[3-(2-{4-[4-Methylpiperazin-1-yl)carbonyl]phenyl}-3H-imidazo[4,5-b]pyridine-7-yl)phenoxy]propyl}acetamide;
4-[3-(2-{4-[(4-Methylpiperazin-1-yl)carbonyl]phenyl}-3-{[2-(trimethylsilyl)ethoxy]methoxy}-3H-imidazo[4,5-b]pyridine-7-yl)phenoxy]butanenitrile;
3-[3-(2-{4-[(4-Methylpiperazin-1-yl)carbonyl]phenyl}-3-{[2-(trimethylsilyl)ethoxy]methoxy}-3H-imidazo[4,5-b]pyridine-7-yl)phenoxy]propan-1-ol;
3-[3-(2-{4-[(4-Methylpiperazin-1-yl)carbonyl]phenyl}-3-{[2-(trimethylsilyl)ethoxy]methoxy}-3H-imidazo[4,5-b]pyridine-7-yl)phenoxy]acetonitrile;
Methyl 6-[7-(4-methoxyphenyl)-3H-imidazo[4,5-b]pyridine-2-yl]nicotinate;
4-[7-(4-Methoxyphenyl)-3H-imidazo[4,5-b]pyridin-2-yl]benzoic acid;
Methyl 4-[7-(4-methoxyphenyl)-3H-imidazo[4,5-b]pyridin-2-yl]pyridine-2-carboxylate;
5-[7-(4-Methoxyphenyl)-3H-imidazo[4,5-b]pyridin-2-yl]nicotinic acid;
4-(7-iodo-3H-imidazo[4,5-b]pyridin-2-yl)benzoic acid;
7-Iodo-2-[4-(3-methoxy propyl-4-ylcarbonyl)phenyl]-3H-imidazo[4,5-b]pyridine;
7-Chloro-2-{4-[(4-methylpiperazin-1-yl)methyl]phenyl}-3H-imidazo[4,5-b]pyridine;
Methyl 4-{2-[4-(morpholin-4-ylmethyl)phenyl]-3H-imidazo[4,5-b]pyridin-7-yl}benzoate;
Methyl 3-[7-(4-methoxyphenyl)-3H-imidazo[4,5-b]pyridin-2-yl]benzoate;
3-[7-(4-Methoxyphenyl)-3H-imidazo[4,5-b]pyridin-2-yl]benzoic acid;
7-Chloro-2-[4-(morpholin-4-ylcarbonyl)phenyl]-3H-imidazo[4,5-b]pyridine;
Methyl 3-(3H-imidazo[4,5-b]pyridin-2-yl)benzoate;
[4-[2-[3-(3-methoxypropoxy)phenyl]-7-(2-trimethylsilylethoxymethyl)-5,7,9-triazabicyclo[4.3.0]nona-1,3,5,8-tetraen-8-yl]phenyl]-(4-methylpiperazin-1-yl)-methanone;
N-[3-[3-[8-[4-(4-methylpiperazin-1-yl)carbonylphenyl]-7-(2-trimethylsilylethoxymethyl)-5,7,9-triazabicyclo[4.3.0]nona-1,3,5,8-tetraen-2-yl]phenoxy]propyl]acetamide;
7-Chloro-2-[4-(morpholin-4-ylmethyl)phenyl]-3H-imidazo[4,5-b]pyridine;
Methyl 3-(7-chloro-3H-imidazo[4,5-b]pyridin-2-yl)benzoate; or
3-{7-[4-(Aminocarbonyl)phenyl]-3H-imidazo[4,5-b]pyridin-2-yl}benzoic acid.
